(19) United States
(12) Patent Application Publication
(10) Pub. No.: US 2025/0340858 A1
(43) Pub. Date: Nov. 6, 2025

(54) COMPOSITIONS AND METHODS FOR PREVENTING DEBRIS LAYER FORMATION DURING PHASE SEPARATION IN AN ATPS-SAMPLE LYSATE MIXTURE

(71) Applicant: Phase Scientific International, Ltd., Hong Kong (CN)

(72) Inventors: Yin To CHIU, Hong Kong (CN); Daniel William BRADBURY, Hong Kong (CN); Garrett Lee MOSLEY, Hong Kong (CN)

(21) Appl. No.: 19/192,392

(22) Filed: Apr. 29, 2025

Related U.S. Application Data
(60) Provisional application No. 63/641,431, filed on May 2, 2024.

(51) Int. Cl. C12N 15/10

(52) U.S. Cl. CPC C12N 15/1003

(57) ABSTRACT

In some embodiments, provided is a method for preventing debris layer formation during phase separation in an ATPS-sample lysate mixture, including the steps of: (a) mixing and incubating a biological sample comprising nucleic acid components and proteins with a lysing composition to form a sample lysate, wherein the composition includes (i) at least one non-ionic surfactant with a hydrophilic-lipophilic balance (HLB) value greater than or equal to 10; and (ii) at least one anionic surfactant, wherein the anionic surfactant comprises a cation and an anionic group; wherein proteins are substantially digested in the sample lysates; (b) adding the sample lysate to an aqueous two-phase system (ATPS) composition to form the ATPS-sample lysate mixture, wherein the ATPS-sample lysate mixture separates into a target-rich phase solution and a target-poor phase solution such that the nucleic acid components partition selectively to the target-rich phase solution without substantial debris layer formation.

### CROSS-REFERENCE TO RELATED APPLICATION

This application claims priority to, and the benefit of, U.S. Provisional Application having Ser. No. 63/641,431 filed on May 2, 2024. The entire contents of the foregoing application are hereby incorporated by reference in its entirety for all purposes.

### FIELD OF INVENTION

This application relates to compositions and methods for preventing debris layer formation. More specifically, the present application relates to methods for preventing debris layer formation during phase separation in an ATPS-sample lysate mixture.

### BACKGROUND OF INVENTION

Aqueous two-phase system (ATPS) can be used to concentrate and isolate target analytes such as nucleic acids from samples. However, other unwanted biomolecules in the samples sometimes aggregate in the ATPS solution and interfere with downstream processes. There is a need for improved methods for processing the samples such that concentration and isolation of target analytes using ATPS can be performed smoothly and efficiently.

### SUMMARY OF INVENTION

Disclosed herein are novel methods for preventing debris layer formation during phase separation in an ATPS-sample lysate mixture, compositions, and kits thereof.

In some embodiments, provided is a method for preventing debris layer formation during phase separation in an ATPS-sample lysate mixture, including the steps of: (a) mixing and incubating a biological sample comprising nucleic acid components and proteins with a lysing composition to form a sample lysate, wherein the composition includes (i) at least one non-ionic surfactant with a hydrophilic-lipophilic balance (HLB) value greater than or equal to 10; and (ii) at least one anionic surfactant, wherein the anionic surfactant comprises a cation and an anionic group; wherein the anionic group is selected from the group consisting of a cholate, a carboxylate, a sulfonate, a sulfate, and a phosphate ester; wherein proteins are substantially digested in the sample lysates; (b) adding the sample lysate to an aqueous two-phase system (ATPS) composition including a polymer, a salt, a surfactant, or any combination thereof dissolved in an aqueous solution to form the ATPS-sample lysate mixture, wherein the ATPS-sample lysate mixture separates into a target-rich phase solution and a target-poor phase solution such that the nucleic acid components partition selectively to the target-rich phase solution without substantial debris layer formation.

In some embodiments, provided is a lysing composition, including: (a) at least one non-ionic surfactant with a hydrophilic-lipophilic balance (HLB) value greater than or equal to 10; and (b) at least one anionic surfactant, wherein the anionic surfactant includes a cation and an anionic group; wherein the anionic group is selected from the group consisting of a cholate, a carboxylate, a sulfonate, a sulfate, and a phosphate ester.

In some embodiments, provided is a kit including (a) the composition of any one of the preceding embodiments; and (b) at least one aqueous two-phase system (ATPS) composition, wherein the at least one aqueous two-phase system (ATPS) includes a polymer, a salt, a surfactant, or any combination thereof.

In some embodiments, provided is a method for preventing debris layer formation during phase separation in an ATPS-sample lysate mixture, including the steps of: (a) mixing and incubating a biological sample including nucleic acid components and proteins with a lysing composition to form a sample lysate, wherein the lysing composition includes: (i) at least one non-ionic surfactant with a hydrophilic-lipophilic balance (HLB) value between 10 and 20; and (ii) at least one anionic surfactant, wherein the anionic surfactant includes a cation and an anionic group; wherein the anionic group is selected from the group consisting of a cholate, a carboxylate, a sulfonate, a sulfate, and a phosphate ester; and the cation is selected from the group consisting of sodium, potassium, ammonium, calcium, lithium, magnesium, aluminum, cesium, barium, straight trimethyl ammonium, branched trimethyl ammonium, triethyl ammonium, tripropyl ammonium, tributyl ammonium, tetramethyl ammonium, tetraethyl ammonium, tetrapropyl ammonium, and tetrabutyl ammonium; wherein the at least one non-ionic surfactant and the at least one anionic surfactant are present in a ratio such that when the at least one non-ionic surfactant and the at least one anionic surfactant are dissolved in a volume of water, the anionic surfactant is present at a concentration of about 0.5-20% (w/v), and the non-ionic surfactant is present at a concentration of about 0.5-20% (w/v), and wherein the proteins are substantially digested in the sample lysate; (b) adding the sample lysate to an aqueous two-phase system (ATPS) composition including a polymer, a salt, a surfactant, or any combination thereof dissolved in an aqueous solution to form the ATPS-sample lysate mixture, wherein the ATPS-sample lysate mixture separates into a target-rich phase solution and a target-poor phase solution such that the nucleic acid components partition selectively to the target-rich phase solution without substantial debris layer formation.

In some embodiments, provided is a lysing composition, including: (a) at least one non-ionic surfactant with a hydrophilic-lipophilic balance (HLB) value between 10 and 20; and (b) at least one anionic surfactant, wherein the anionic surfactant includes a cation and an anionic group; wherein the anionic group is selected from the group consisting of a cholate, a carboxylate, a sulfonate, a sulfate, and a phosphate ester; and the cation is selected from the group consisting of sodium, potassium, ammonium, calcium, lithium, magnesium, aluminum, cesium, barium, straight trimethyl ammonium, branched trimethyl ammonium, triethyl ammonium, tripropyl ammonium, tributyl ammonium, tetramethyl ammonium, tetraethyl ammonium, tetrapropyl ammonium, and tetrabutyl ammonium, wherein the at least one non-ionic surfactant and the at least one anionic surfactant are present in a ratio such that when the at least one non-ionic surfactant and the at least one anionic surfactant are dissolved in a volume of water, the anionic surfactant is present at a concentration of about 0.5-20% (w/v), and the non-ionic surfactant is present at a concentration of about 0.5-20% (w/v).

### Advantages

There are many advantages to the various embodiments of the present disclosure. In certain embodiments, the methods, compositions and kits of the present disclosure provide simple and effective means to digest protein in the biological samples and to prevent precipitation of proteins during downstream extraction process using ATPS.

Controlling interface layer formation in the ATPS can have an impact on recovery and ease of automation. When adding sample lysates to the ATPS, unwanted proteins released from lysed cells will oftentimes aggregate and precipitate in the interface layer, forming a debris layer in the ATPS solution that is hard to remove. If the resulting debris layer is stuck to a pipette tip, it may clog or cause high protein/surfactant carry over into the subsequent steps (such as the next ATPS), and may interfere with downstream analytical or purification procedures if not effectively managed or removed. In some embodiments, it is surprisingly found that using the methods disclosed herein, which involve the lysis of the biological samples using the disclosed compositions prior to adding the sample lysates to the ATPS, the debris layer formation in the interface layer is surprisingly and effectively prevented during phase separation in the ATPS-sample lysate mixture.

In some embodiments, the extraction and recovery efficiency of a target analyte (e.g. nucleic acids) using the disclosed methods and kits is surprisingly higher when compared to the extraction and recovery efficiency using methods without the prior lysis step with the disclosed compositions. In some embodiments, the methods described herein surprisingly and effectively prevent debris layer formation. The methods can easily be incorporated into fully automated extraction workflows.

In some embodiments, the compositions disclosed herein include combinations of at least one anionic surfactant with at least one non-ionic surfactant having a hydrophilic-lipophilic balance (HLB) value greater than or equal to 10. The combinations described herein surprisingly allow the proteins in the biological samples to be digested without substantially forming debris layers during the downstream extraction process using ATPS. The problem with anionic surfactants is that they have more limited solubility in high salt environments such as ATPS due to salt-charge screening which significantly reduces the hydrophilicity of the hydrophilic head and overall surfactant/micelle solubility. The combination of an anionic surfactant with at least one non-ionic surfactant having a high HLB (such as non-ionic surfactants with a hydrophilic-lipophilic balance (HLB) value greater than or equal to 10) surprisingly and significantly reduces precipitation of proteins during the extraction process using ATPS. In some embodiments, the compositions of the present disclosure comprise a mixture of anionic:nonionic micelles which are more robust and soluble in a high salt environment. In some embodiments, the overall micelle/surfactant solubility is less affected by salt charge screening. In some embodiments, these mixed micelles surprisingly reduce protein/surfactant interface precipitation within the ATPS.

In some embodiments, the methods and kits disclosed herein can effectively concentrate and purify target analytes that are present at very low concentrations in the biological samples, such as cell-free DNA (cfDNA), removing unwanted proteins that might interfere with the downstream detection.

These and other features and characteristics, as well as the methods of use, and functions of the related components, will become more apparent upon consideration of the following detailed description and the appended claims with reference to the accompanying figures, all of which form a part of this specification, wherein like reference numerals designate corresponding parts in the various figures. It is to be expressly understood, however, that the drawings are for the purpose of illustration and description only and are not intended as a definition of the limits of the claims.

### BRIEF DESCRIPTION OF FIGURES

FIGS. 1A, 1B, 1C and 1D show the ATPS-sample lysate mixtures after phase separation in the first ATPS according to example embodiments, in which the sample lysates were prepared from example lysis buffer compositions according to Table 2.

FIG. 2A is a graph showing the Ct values of 145 bp dsDNA recovered from the sample lysates using ATPS according to example embodiments, in which the sample lysates were prepared from selected lysis buffer compositions according to Table 5.

FIG. 2B is a graph showing the Qubit readings of dsDNA recovered from the sample lysates using ATPS according to example embodiments, in which the sample lysates were prepared from selected lysis buffer compositions according to Table 6.

### DETAILED DESCRIPTION

As used herein and in the claims, the terms “comprising” (or any related form such as “comprise” and “comprises”), “including” (or any related forms such as “include” or “includes”), “containing” (or any related forms such as “contain” or “contains”), means including the following elements but not excluding others. It shall be understood that for every embodiment in which the term “comprising” (or any related form such as “comprise” and “comprises”), “including” (or any related forms such as “include” or “includes”), or “containing” (or any related forms such as “contain” or “contains”) is used, this disclosure/application also includes alternate embodiments where the term “comprising”, “including,” or “containing,” is replaced with “consisting essentially of” or “consisting of”. These alternate embodiments that use “consisting of” or “consisting essentially of” are understood to be narrower embodiments of the “comprising”, “including,” or “containing,” embodiments.

For example, alternate embodiments of “a composition comprising A, B, and C” would be “a composition consisting of A, B, and C” and “a composition consisting essentially of A, B, and C.” Even if the latter two embodiments are not explicitly written out, this disclosure/application includes those embodiments. Furthermore, it shall be understood that the scopes of the three embodiments listed above are different.

For the sake of clarity, “comprising”, including, and “containing”, and any related forms are open-ended terms which allows for additional elements or features beyond the named essential elements, whereas “consisting of” is a closed end term that is limited to the elements recited in the claim and excludes any element, step, or ingredient not specified in the claim.

For the sake of clarity, “characterized by” or “characterized in” (together with their related forms as described above), does not limit or change the nature of whether the list of terms following it are open or closed. For example, in a claim directed towards “a composition comprising A, B, C, and characterized in D, E, and F”, the elements D, E, and F are still open-ended terms and the claim is meant to include other elements due to the use of the word “comprising” earlier in the claim.

“Consisting essentially of” limits the scope of a claim to the specified materials, components, or steps (“essential elements”) that do not materially affect the essential characteristic(s) of the claimed invention. In some embodiments, the essential characteristics are the basic and novel characteristic(s) of the claimed invention. For example, in some embodiments, the essential elements of a composition of the disclosure can be “Xmg to Ymg” of compound A. Even if the composition includes additional excipients, as long as the additional excipients do not materially affect the essential characteristics of the compound, e.g., in compound A's ability to bind to XX target or to treat YY disease, then such embodiment that “consists essentially of compound A” still includes compositions with the aforementioned additional excipients.

As used herein, the singular forms “a”, “an” and “the” are intended to include the plural forms as well, unless the context clearly indicates otherwise.

Where a range is referred in the specification, the range is understood to include each discrete point within the range. For example, 1-7 means 1, 2, 3, 4, 5, 6, and 7.

As used herein, the term “about” is understood as within a range of normal tolerance in the art and not more than +20% of a stated value. By way of example only, about 50 means from 45 to 55 including all values in between. As used herein, the phrase “about” a specific value also includes the specific value, for example, about 50 includes 50.

As used herein and in the claims, “lysis debris layer” or “debris layer” refers to precipitation that occurs as a result of agglomeration or condensation of macromolecules, typically during biochemical processes. In some embodiments, a debris layer forms when proteins released from lysed cells aggregate and precipitate out of solution, often due to changes in environmental conditions such as pH, temperature, or the presence of precipitating agents. In some embodiments, this precipitation forms at the interlayer between two phases of an ATPS.

As used herein and in the claims, “without substantial debris layer formation” means the volume of the debris layer formed (if any) is at least 50% less than the volume of debris layer formed in an ATPS that does not utilize the lysing composition as described herein.

As used herein and in the claims, “hydrophilic-lipophilic balance” (HLB) value refers to the degree of hydrophilicity or lipophilicity of a non-ionic surfactant. The HLB value is determined based on the ratio between the molecular mass of the hydrophilic portion of the surfactant molecule and its total molecular mass. For example, according to the Griffin method, an HLB value is equal to the molecular weight percentage of the hydrophilic portion (with respect to the whole molecule) divided by 5.

The term “hydrophilic” refers to the capacity of a molecule or portion of a molecule to interact with polar solvents, in particular with water, or with other polar moieties driven by hydrogen bonding, dipole-ion interactions and/or dipole-dipole interactions.

The terms “lipophilic” and “hydrophobic” can be used interchangeably and refer to the tendency of a molecule or portion of a molecule to dissolve in non-polar environment such as fats, oils, and non-polar solvents driven by London dispersion forces.

As used herein, a “surfactant” includes, but is not limited to, an anionic surfactant, nonionic surfactant, cationic surfactant, zwitterionic surfactant or amphoteric surfactant.

In some embodiments, the anionic surfactant includes a cation and an anionic group. In some embodiments, the cation is selected from the group consisting of sodium, potassium, ammonium, calcium, lithium, magnesium, aluminum, cesium, barium, straight trimethyl ammonium, branched trimethyl ammonium, triethyl ammonium, tripropyl ammonium, tributyl ammonium, tetramethyl ammonium, tetraethyl ammonium, tetrapropyl ammonium, tetrabutyl ammonium, and any combination thereof. In some embodiments, the anionic group is selected from the group consisting of a cholate, a carboxylate, a sulfonate (also referred as ‘sulphonate’), a sulfate (also referred as ‘sulphate’), and a phosphate ester. For the sake of clarity, when it is described in the specification and the claims that the anionic group is selected from the group consisting of functional groups (for example, a cholate, a carboxylate, a sulfonate, a sulfate, and a phosphate ester), it shall be understood that the anionic group includes one of the functional group as a part of its chemical structure, but is not limited to the functional group as its entire chemical structure. For example, a carboxylate could be a simple carboxylate such as —C(═O)(O) or an alkyl ether carboxylate.

As used herein, a “sulfonate” refers to an anionic compound characterized by the presence of at least one sulfonate group (—SO₃⁻) that is covalently bonded to a hydrophobic moiety. Examples of sulfonates include, but are not limited to, petroleum sulfonates, alkylbenzenesulfonates (such as dodecyl benzene sulfonate), naphthalenesulfonates, and olefin sulfonates. For example, one of skilled in the art would understand that alkylbenzenesulfonates is a class of anionic surfactants of different carbon chain sizes.

A hydrophobic moiety refers to a carbon-based structure comprising a moiety of saturated or unsaturated hydrocarbons (alkanes, alkenes, alkynes), aromatic groups, ethers, carbonyls, carboxyls, esters, alcohols, amines, imines, amides, phenyls, or combinations thereof. In some embodiments, a hydrophobic moiety has a range of 1-40 carbon atoms. In some embodiments, a long-chain moiety is linear, branched, cyclic, or spirocyclic. In some embodiments, examples of a hydrophobic moiety include, but not limited to, alkyls (such as dodecyl), natural oils, natural fats, esters, alkanolamides, and alkylphenols.

As used herein, a “sulfate” refers to an anionic compound characterized by the presence of at least one sulfate group (—SO₄²⁻) that is covalently bonded to a hydrophobic moiety. Examples of sulfates include, but are not limited to, alkyl sulfates (such as dodecyl sulfate), sulfated natural oils, sulfated natural fats, sulfated esters, sulfated alkanolamides, and sulfated alkylphenols. In some embodiments, natural oils include fish oils, vegetable oils, such as castor oil, olive oil, rapeseed oil, and the like.

As used herein, a “carboxylate” refers to an anionic compound characterized by the presence of at least one carboxylate group (—CO₂⁻) that is covalently bonded to a hydrophobic moiety. Examples of carboxylates include, but are not limited to, alkyl sarcosinates (such as N-lauroyl sarcosinate, cocoyl sarcosinate, myristoyl sarcosinate, oleoyl sarcosinate, and stearoyl sarcosinate), laureth-11 carboxylic acid, laureth-5 carboxylic acid, trideceth-7 carboxylic acid, sodium myristate, potassium dodecanoate, sodium laurate, sodium oleates, palmitates, and stearates.

As used herein, a “cholate” refers to an anionic compound characterized by the presence of the moiety composed of a steroid structure with four rings and a 5-8 carbon side-chain. Examples of cholates include, but are not limited to, cholate, glycocholate, taurocholate, deoxycholate, and chenodeoxycholate.

As used herein, a “phosphate ester” refers to an anionic compound characterized by the presence of at least one phosphate group (PO₄²⁻) with one of the oxygens covalently bonded to a hydrophobic moiety, or the presence of (PO₃OR)²⁻, where R is a hydrophobic moiety. Examples of phosphate esters include, but are not limited to, alkoxylated alcohol phosphate esters, and polyfluorinated alkyl phosphate esters.

Examples of non-ionic surfactants include, but are not limited to, ethoxylated alkylphenol, ethoxylated natural fatty alcohol, octylphenoxy alcohol, ethoxylated sorbitan ester, ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, and polyoxyethylene fatty acid amides.

Examples of octylphenoxy alcohol include, but are not limited to, polyethylene glycol octylphenyl ethers, polyethylene glycol tert-octylphenyl ethers, octylphenoxypolyethoxyethanols, or (such as Triton X-100, Triton X-114, Triton X-45, or Igepal CA630). In some embodiments, octylphenoxy alcohol comprises compounds having the formula of

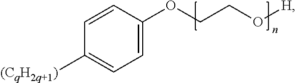

wherein n is 4-40 and q is 6-12. In some embodiments, octylphenoxy alcohol comprises a compound having the formula of

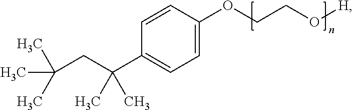

wherein n is 9-10 (such as Triton X-100).

In some embodiments, the octylphenoxy alcohol has an average molecular weight of about 400-2000 g/mol. In some embodiments, the octylphenoxy alcohol has an average molecular weight of about 600-2000 g/mol. In some embodiments, the octylphenoxy alcohol has an average molecular weight of about 603 g/mol, about 617 g/mol, about 735 g/mol. In some embodiments, the non-ionic surfactant has an average molecular weight of about 537 g/mol, about 625 g/mol, about 911 g/mol or about 1967 g/mol.

Examples of ethoxylated sorbitan ester include, but are not limited to, polyoxyethylene (20) sorbitan monolaurate, polyoxyethylene (20) sorbitan monopalmitate, polyoxyethylene (20) sorbitan monostearate, polyoxyethylene (20) sorbitan monooleate. In some embodiments, ethoxylated sorbitan ester comprises compounds having the formula of

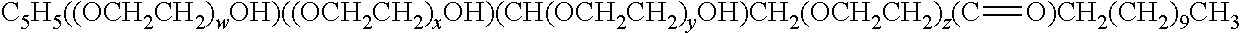

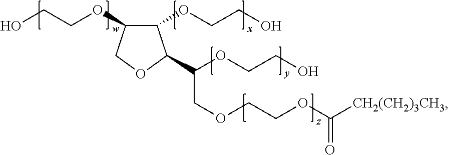

wherein w+x+y+z=20 (such as Tween 20).

Examples of ethoxylated natural fatty alcohol include, but are not limited to, polyoxyethylene (23) lauryl ether, polyoxyethylene (20) cetyl ether and polyoxyethylene (10) oleyl ether (such as Brij L23, Brij 58, Brij O10). In some embodiments, the non-ionic surfactant has an average molecular weight of about 1198 g/mol, about 1124 g/mol, or about 708 g/mol. In some embodiments, ethoxylated natural fatty alcohol comprises compounds having the formula of CH₃(CH₂)₁₀CH₂(OCH₂CH₂)ₙOH, wherein n is 23 (such as Brij 23). In some embodiments, n is 10.

Examples of cationic surfactants include, but are not limited to, quaternary ammonium salts, amines with amide linkages, polyoxyethylene alkyl amines, polyoxyethylene alicyclic amines, n,n,n′,n′ tetrakis substituted ethylenediamines, and 2-alkyl 1-hydroxethyl 2-imidazolines.

Examples of amphoteric surfactants include, but are not limited to, n-coco 3-aminopropionic acid or sodium salt thereof, n-tallow 3-iminodipropionate or disodium salt thereof, n-carboxymethyl n dimethyl n-9 octadecenyl ammonium hydroxide, n-cocoamidethyl n hydroxyethylglycineor sodium salt thereof, and sodium N-lauroyl sarcosinate (NLS).

In some embodiments, the surfactant comprises a polymer such as PAG. In some embodiments, the surfactant has a structure of EOₓ-PO_{y}-EOₓ, wherein EO refers to an ethylene oxide unit and PO refers to a propylene oxide unit, and x and y are the respective number of monomers. In some embodiments, x=2-136. In some embodiments, y=16-62. In some embodiments, examples of surfactants include, but are not limited to, (C₂H₄O)ₙC₁₄H₂₂O wherein n=4-10 (such as Triton X-100, Triton X-114, Triton X-45, Tween 20, Igepal CA630), Brij 58, Brij O10, Brij L23, EOₓ-PO_{y}-EOₓ wherein x=2-136 and y=16-62 (such as Pluronic L-61, Pluronic F-127), sodium dodecyl sulfate, sodium cholate, sodium deoxycholate, N-lauroyl sarcosine sodium salt (NLS), hexadecyltrimethylammonium bromide, or span 80.

“Aqueous,” as used herein, refers to the characteristic properties of a solvent/solute system wherein the solvating substance has a predominantly hydrophilic character. Examples of aqueous solvent/solute systems include those where water, or compositions containing water, are the predominant solvent. The polymer and/or surfactant components whose use is described in the embodiments are “aqueous” in the sense that they form aqueous phases when combined with a solvent such as water. Further, as understood by the skilled person, in the present context the term liquid “mixture” refers merely to a combination of the herein-defined components.

As used herein, an aqueous two-phase system (ATPS) means a liquid-liquid separation system that can accomplish isolation or concentration of an analyte by partitioning, where two phases, sections, areas, components, or the like, interact differently with at least one analyte to which they are exposed and optionally dissolved. An ATPS is formed when two immiscible phase forming components, such as a salt and polymer, or two incompatible polymers (e.g., PEG and dextran) with certain concentrations are mixed in an aqueous solution. ATPS methods are relatively inexpensive and scalable because they employ two-phase partitioning to separate analytes (e.g., nucleic acids) from contaminants.

The term “isolated” as used herein refers to an analyte being removed from its original environment and thus is altered from its original environment. For example, an isolated nucleic acid generally is provided with fewer non-nucleic acid components (e.g., protein, lipid) than the amount of components present in a source sample. A composition comprising an isolated analyte, (e.g., sample nucleic acid) can be substantially isolated (e.g., about 90%, 91%, 92%, 93%, 94%, 95%, 96%, 97%, 98%, 99% or greater than 99% free of non-analyte components (such as non-nucleic acid components)).

As used herein, “concentrated” means that the mass ratio of analyte in question to the solution in which the analyte is suspended is higher than the mass ratio of said analyte in its pre-concentration solution. It can, for example, be slightly higher, or more preferably at least twice, ten times or one hundred times as high.

As used herein, the term “polymer” refers to any polymer including at least one substituted or non-substituted monomer. Examples of polymer includes, but are not limited to, homopolymer, copolymer, terpolymer, random copolymer, and block copolymer. Block copolymers include, but are not limited to, block, graft, dendrimer, and star polymers. As used herein, copolymer refers to a polymer derived from two monomeric species; similarly, a terpolymer refers to a polymer derived from three monomeric species. The two or three monomeric species can be the same or different species. The polymer also includes various morphologies, including, but not limited to, linear polymer, branched polymer, random polymer, crosslinked polymer, and dendrimer systems. In some embodiments, a polymer also includes its chemically modified equivalent, such as hydrophobically-modified, or silicone-modified. As an example, polyacrylamide polymer refers to any polymer including at least one substituted or non-substituted acrylamide unit, e.g., a homopolymer, copolymer, terpolymer, random copolymer, block copolymer or terpolymer of polyacrylamide; polyacrylamide can be a linear polymer, branched polymer, random polymer, crosslinked polymer, or a dendrimer of polyacrylamide; polyacrylamide can be hydrophobically-modified polyacrylamide, or silicone-modified polyacrylamide.

In some embodiments, examples of polymer include, but are not limited to, polyethers, polyimines, polyacrylates, polyalkylene glycols, vinyl polymers, alkoxylated surfactants, polysaccharides, polyether-modified silicones, polyacrylamide, polyacrylic acids and copolymers thereof. In some embodiments, the polymer is hydrophobically-modified, or silicone-modified. In some embodiments, the polymer is polyvinyl methylether, hydroxypropyl cellulose, or polyethyleneimine. In some embodiments, the polymer is PEG-PPG, polyvinyl pyrrolidone, or methyl cellulose. In some embodiments, the polymer is polyethylene glycol, polypropylene glycol, or dextran. In some embodiments, the polymer is alkylbenzenesulfonates, polyacrylamide, or isopropylacrylamide. In some embodiments, the polymer is acrylamide. In some embodiments, the polymer is dextran. In some embodiments, the polymer is polyalkylene glycols.

Examples of polyalkylene glycols (also referred as ‘PAG’ or ‘poly(oxyalkylene)’ or ‘poly(alkylene oxide)’) include, but are not limited to, hydrophobically modified polyalkylene glycols, poly(oxyalkylene) polymer, poly(oxyalkylene) copolymer, hydrophobically modified poly(oxyalkylene) copolymers, dipropylene glycol, tripropylene glycol, polyethylene glycol (also referred as ‘PEG’), polypropylene glycol (also referred as ‘PPG’). In some embodiments, examples of copolymers of PAGs include, but are not limited to, poly(ethylene glycol-propylene glycol) (also referred as ‘PEG-PPG’ or ‘UCON’), and poly(ethylene glycol-ran-propylene glycol) (also referred as ‘PEG-ran-PPG’). In some embodiments, PEG-PPG comprises random copolymers, block copolymers, or combination thereof. In some embodiments, PEG-PPG comprise both random copolymers and block copolymers. In some embodiments, PEG-PPG is PEG-ran-PPG.

As used herein, “vinyl polymer” refers to a group of polymers derived from substituted vinyl (H₂C═CHR) monomers. Examples of vinyl polymer include, but are not limited to, polyvinyl pyrrolidone, polyvinyl alcohol, polyvinyl caprolactam, and polyvinyl methylether.

Examples of polysaccharides include, but are not limited to, dextran, carboxymethyl dextran, dextran sulfate, hydroxypropyl dextran, starch, carboxymethyl cellulose, hydroxypropyl cellulose, methyl cellulose, ethylhydroxyethylcellulose, and maltodextrin. In some embodiments, polysaccharides are alkoxylated starches, alkoxylated cellulose, or alkyl hydroxyalkyl cellulose.

Examples of polyacrylamide include, but are not limited to, poly N-isopropylacrylamide.

Examples of polyimines include, but are not limited to, polyethyleneimine.

As used herein, the term “salt” refers to a substance having at least one cation and at least one anion. Examples of salts include, but are not limited to, salts wherein the cation is sodium, potassium, calcium, lithium, magnesium, aluminum, cesium, barium, straight trimethyl ammonium, branched trimethyl ammonium, triethyl ammonium, tripropyl ammonium, tributyl ammonium, tetramethyl ammonium, tetraethyl ammonium, tetrapropyl ammonium, or tetrabutyl ammonium, and/or wherein the anion is phosphate, hydrogen phosphate, dihydrogen phosphate, sulfate, sulfide, sulfite, hydrogen sulfate, carbonate, hydrogen carbonate, acetate, nitrate, nitrite, sulfite, chloride, fluoride, chlorate, perchlorate, chlorite, hypochlorite, bromide, bromate, hypobromite, iodide, iodate, cyanate, thiocyanate, isothiocyanate, oxalate, formate, chromate, dichromate, permanganate, polyacrylate, hydroxide, hydride, citrate, borate, or tris. In some embodiments, the salts are kosmotropic salts, chaotropic salts, or inorganic salts.

As used herein, the term “biological sample” refers to any sample obtained directly or indirectly from a subject (e.g., a human). In some embodiments the subject is a human patient. Examples of clinical biological samples include but are not limited to blood, plasma, urine, saliva, stool, cerebrospinal fluid (CSF), lymph, serum, sputum, peritoneal fluid, sweat, tears, nasal swab, vaginal swab, endocervical swab, semen, breast milk, and other bodily fluids. In some embodiments, the clinical biological sample is obtained from a biological sample treated with a lysing composition.

As used herein and in the claims, “nucleic acid components” refers to deoxyribonucleic acid (DNA), ribonucleic acid (RNA), synthetic oligonucleotides, synthetic nucleic acid analogs or any combination thereof. In some embodiments, the DNA or RNA are DNA fragments or RNA fragments respectively. In some embodiments, examples of nucleic acid components include, but are not limited to, genomic DNA (gDNA), cDNA, plasmid DNA, mitochondrial DNA, cell-free DNA (cfDNA), circulating tumor DNA (ctDNA), circulating fetal DNA, cell-free microbial DNA, micro RNA (miRNA), messenger RNA (mRNA), transfer RNA (tRNA), ribosomal RNA (rRNA), circular RNA, long non-coding RNA (lncRNA), small interfering RNA (siRNA) or combinations thereof.

As used herein, “cell-free DNA” (cfDNA) is DNA that is present outside a cell, e.g., DNA present in the sample (e.g. blood, plasma, serum, or urine) obtained from a subject.

As used herein, the term “Ct”, “CT”, “Ct value” or “CT value” refers to a cycle threshold value and signifies the cycle of a PCR amplification assay in which signal from a reporter that is indicative of amplicon generation (e.g., fluorescence) first becomes detectable above a background level. In some embodiments, the CT value is an indirect indicator of the amount of target nucleic acid detected from a particular sample. In general, a lower CT value indicates a higher amount of the target nucleic acid in the sample, and a higher CT value indicates a lower amount of the target nucleic acid in the sample.

As used herein, the term “purification system” refers to a device, a product, a method or a process that is used to purify and/or selectively isolate a target analyte by chemical or physical means. In some embodiments, a purification system comprises a solid phase medium. In some embodiments, a purification system comprises a magnetic beads workflow or a spin column workflow. In some embodiments, a purification system comprises a solid phase extraction column, liquid chromatography column, or magnetic beads.

As used herein, the term “solid phase medium” refers to a material which selectively isolates a target analyte by chemical or physical means. In some embodiments, a solid phase medium is provided as a solid, or a solid supported in a vessel. In some embodiments, the solid phase medium is a solid phase extraction column, such as a spin column. In some embodiments, the solid phase medium is a plurality of beads, silica resins, silica membrane, silica gel, alumina gel, size exclusion resins, or ion-exchange resins.

It shall be understood to one of skill in the art that letters in the formulae such as n, w, x, y, and z refer to whole numbers and describe the number of repeating units in the bracketed region of each figure. For example, if n is 2 for repeating unit of —CH₂CH₂O— (as shown in formula for Tween 20, for example), this refers to a chain with two ethylene units as shown below: —CH₂CH₂OCH₂CH₂O—.

For the sake of clarity, when describing a compound having a formula wherein “n is X or Y or a mixture thereof”, the term “a mixture thereof” refers to a mixture of compounds of said formula, wherein some of the compounds have n=X and some of the compounds have n=Y. The mixture can be different ratios of compounds with n=X and compounds with n=Y, including, but not limited to 1:1, 1:2, 1:3, 1:4, 1:8, 1:10, 2:1 4:1, 8:1, 10:1, etc.

In various aspects, the compounds disclosed herein further comprise their isotopically-labelled or isotopically-substituted variants, i.e., compounds identical to those described, but for the fact that one or more atoms are replaced by an atom having an atomic mass or mass number different from the atomic mass or mass number typically found in nature. For example, the isotopically-labelled or isotopically-substituted atom has one or more neutrons in the nucleus compared to the natural atom. In some embodiments, the disclosed compounds comprise a mixture of natural atoms and their isotopically labeled variants. Examples of isotopes that can be incorporated into the disclosed compounds include isotopes of hydrogen, carbon, nitrogen, oxygen, sulfur, phosphorous, fluorine and chlorine, such as ²H, ³H, ¹³C, ¹⁴C, ¹⁵N, ¹⁷O, ¹⁸O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this disclosure.

Although the description referred to particular embodiments, the disclosure should not be construed as limited to the embodiments set forth herein.

In some embodiments, provided is a method including the steps of: (a) mixing and incubating a biological sample including nucleic acid components and proteins with a lysing composition to form a sample lysate, wherein the composition includes: (i) at least one non-ionic surfactant; and (ii) at least one anionic surfactant, wherein the anionic surfactant includes a cation and an anionic group; wherein the anionic group is selected from the group consisting of a cholate, a carboxylate, a sulfonate, a sulfate, and a phosphate ester; wherein proteins are substantially digested in the sample lysates; (b) adding the sample lysate to an aqueous two-phase system (ATPS) composition including a polymer, a salt, a surfactant, or any combination thereof dissolved in an aqueous solution to form the ATPS-sample lysate mixture, wherein the ATPS-sample lysate mixture separates into a target-rich phase solution and a target-poor phase solution such that the nucleic acid components partition selectively to the target-rich phase solution without substantial debris layer formation.

In some embodiments, provided is a method for preventing debris layer formation during phase separation in an ATPS-sample lysate mixture, including the steps of: (a) mixing and incubating a biological sample including nucleic acid components and proteins with a lysing composition to form a sample lysate, wherein the composition includes: (i) at least one non-ionic surfactant; and (ii) at least one anionic surfactant, wherein the anionic surfactant includes a cation and an anionic group; wherein the anionic group is selected from the group consisting of a cholate, a carboxylate, a sulfonate, a sulfate, and a phosphate ester; wherein proteins are substantially digested in the sample lysates; (b) adding the sample lysate to an aqueous two-phase system (ATPS) composition including a polymer, a salt, a surfactant, or any combination thereof dissolved in an aqueous solution to form the ATPS-sample lysate mixture, wherein the ATPS-sample lysate mixture separates into a target-rich phase solution and a target-poor phase solution such that the nucleic acid components partition selectively to the target-rich phase solution without substantial debris layer formation.

In some embodiments, provided is a method for preventing debris layer formation during phase separation in an ATPS-sample lysate mixture, including the steps of: (a) mixing and incubating a biological sample including nucleic acid components and proteins with a lysing composition to form a sample lysate, wherein the composition includes: (i) at least one non-ionic surfactant with a hydrophilic-lipophilic balance (HLB) value greater than or equal to 10; and (ii) at least one anionic surfactant, wherein the anionic surfactant includes a cation and an anionic group; wherein the anionic group is selected from the group consisting of a cholate, a carboxylate, a sulfonate, a sulfate, and a phosphate ester; wherein proteins are substantially digested in the sample lysates; (b) adding the sample lysate to an aqueous two-phase system (ATPS) composition including a polymer, a salt, a surfactant, or any combination thereof dissolved in an aqueous solution to form the ATPS-sample lysate mixture, wherein the ATPS-sample lysate mixture separates into a target-rich phase solution and a target-poor phase solution such that the nucleic acid components partition selectively to the target-rich phase solution without substantial debris layer formation.

In some embodiments, the method further includes the steps of (c) optionally collecting the first target-rich phase solution and mixing the first target-rich phase solution with a second ATPS composition including a polymer, a salt, a surfactant, or any combination thereof dissolved in an aqueous solution to form a second target-rich phase solution and a second target-poor phase solution, such that the nucleic acid components partition to the second target-rich phase solution without substantial debris layer formation; and (d) isolating the nucleic acid components from the target-rich phase solution and/or the second target-rich phase solution.

In some embodiments, step (d) further includes the following steps: mixing the target-rich phase solution or the second target-rich phase solution with a binding buffer to form a mixed solution; loading the mixed solution onto a purification system configured to selectively extract and purify the nucleic acid components; and eluting and collecting the nucleic acid components from the purification system, resulting in a final solution containing the concentrated and purified nucleic acid components.

In some embodiments, the purification system is a solid phase extraction column, liquid chromatography column, or magnetic beads.

In some embodiments, the nucleic acid components are DNA, RNA, or any combination thereof.

In some embodiments, the nucleic acid components are genomic DNA, cDNA, plasmid DNA, cell-free DNA, circulating tumor DNA, micro RNA, messenger RNA, transfer RNA, ribosomal RNA, long non-coding RNA, siRNA, or any combination.

In some embodiments, the nucleic acid components are cell-free DNA.

In some embodiments, the binding buffer includes a chaotropic agent selected from the group consisting of guanidinium hydrochloride (GHCl), guanidinium thiocyanate, guanidinium isothiocyanate (GITC)), sodium thiocyanate, sodium iodide, sodium perchlorate, sodium trichloroacetate, sodium trifluoroacetate, lithium perchlorate, lithium acetate, magnesium chloride, phenol, 2-propanol, thiourea, and urea.

In some embodiments, the chaotropic agent is present at a concentration of 2M to 7M.

In some embodiments, the binding buffer further includes a polymer at a concentration of 5-20% (w/v).

In some embodiments, the binding buffer includes guanidine thiocyanate and PEG, and the purification system is magnetic beads.

In some embodiments, the biological sample is selected from the group consisting of blood, plasma, urine, saliva, stool, cerebrospinal fluid (CSF), lymph, serum, sputum, peritoneal fluid, sweat, tears, nasal swab, vaginal swab, endocervical swab, semen, or breast milk.

In some embodiments, the composition consists essentially of one non-ionic surfactant and one anionic surfactant.

In some embodiments, provided is a lysing composition, including: (a) at least one non-ionic surfactant; and (b) at least one anionic surfactant, wherein the anionic surfactant includes a cation and an anionic group; wherein the anionic group is selected from the group consisting of a cholate, a carboxylate, a sulfonate, a sulfate, and a phosphate ester.

In some embodiments, provided is a lysing composition, including: (a) at least one non-ionic surfactant with a hydrophilic-lipophilic balance (HLB) value greater than or equal to 10; and (b) at least one anionic surfactant, wherein the anionic surfactant includes a cation and an anionic group; wherein the anionic group is selected from the group consisting of a cholate, a carboxylate, a sulfonate, a sulfate, and a phosphate ester.

In some embodiments, the hydrophilic-lipophilic balance (HLB) value of the at least one non-ionic surfactant is between 10 and 20.

In some embodiments, the hydrophilic-lipophilic balance (HLB) value of the at least one non-ionic surfactant is between 10 and 17.

In some embodiments, the hydrophilic-lipophilic balance (HLB) value of the at least one non-ionic surfactant is between 12.4 and 16.9.

In some embodiments, the hydrophilic-lipophilic balance (HLB) value of the at least one non-ionic surfactant is 12.4, 13.4, 16.7 or 16.9.

In some embodiments, the at least one non-ionic surfactant includes at least one (OCH₂CH₂)ₙ— chain and at least one OH group, wherein n is a whole number between 2 and 40.

In some embodiments, the at least one non-ionic surfactant is selected from the group consisting of ethoxylated natural fatty alcohol, octylphenoxy alcohol, ethoxylated sorbitan ester, and any combination thereof.

In some embodiments, the ethoxylated natural fatty alcohol is a compound of Formula III: CH₃(CH₂)₁₀CH₂(OCH₂CH₂)ₙOH (Formula III), wherein n is 10-23. In some embodiments, the ethoxylated natural fatty alcohol is a compound of Formula III: CH₃(CH₂)₁₀CH₂(OCH₂CH₂)ₙOH (Formula III), wherein n is 10-25.

In some embodiments, the octylphenoxy alcohol includes one or more compounds of Formula I:

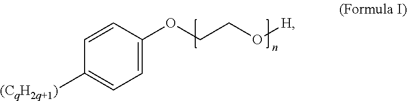

wherein n is 6, 7, 8, 9, or 10, and q is 6, 7, 8, 9, 10, 11, or 12.

For the sake of clarity, when it is described herein that “n is x and q is y”, in which x and y are one or more integers, it shall be understood that each n and each q value is independent of any other values. For example, a compound of Formula I “wherein n is 6, 7, 8, 9, or 10, and q is 6, 7, 8, 9, 10, 11, or 12”, refers to a group of compounds wherein for each compound, n is independently 6, 7, 8, 9, or 10, and q is independently 6, 7, 8, 9, 10, 11, or 12”. As such, in some embodiments the group of compounds included in a particular Formula includes mixtures of compounds where n and/or q are present in different values.

In some embodiments, n is 7 or 8 or a mixture thereof, and q is 8; or n is 9 or 10 or a mixture thereof, and q is 8.

In some embodiments, n is 9, 10, or a mixture thereof; and the average molecular weight of the octylphenoxy alcohol is about 603 g/mol or about 625 g/mol.

In some embodiments, the ethoxylated sorbitan ester is a compound of Formula II:

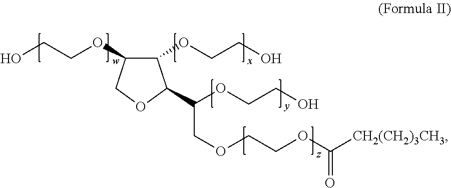

wherein w+x+y+z=20.

In some embodiments, the at least one non-ionic surfactant is selected from the group consisting of Triton X-100, Triton X-114, Brij L23, Tween 20 and IGEPAL CA630.

In some embodiments, the sulfonate is selected from the group consisting of petroleum sulfonate, alkylbenzenesulfonate, naphthalenesulfonate, olefin sulfonate, and any combination thereof; and wherein the sulfate is selected from the group consisting of alkyl sulfate, sulfated natural oil, sulfated natural fat, sulfated ester, sulfated alkanolamide, sulfated alkylphenol, and any combination thereof.

In some embodiments, the cholate is sodium cholate, the carboxylate is sodium N-lauroyl sarcosine (NLS), the sulfate is sodium dodecyl sulfate (SDS), and the sulfonate is sodium dodecyl benzene sulfonate (SDBS).

In some embodiments, the cation is selected from the group consisting of sodium, potassium, ammonium, calcium, lithium, magnesium, aluminum, cesium, barium, straight trimethyl ammonium, branched trimethyl ammonium, triethyl ammonium, tripropyl ammonium, tributyl ammonium, tetramethyl ammonium, tetraethyl ammonium, tetrapropyl ammonium, tetrabutyl ammonium, and any combination thereof. In some embodiments, the cation is selected from the group consisting of sodium, potassium, ammonium, and any combination thereof.

In some embodiments, the at least one anionic surfactant is selected from the group consisting of sodium cholate, sodium N-lauroyl sarcosine (NLS), sodium dodecyl sulfate (SDS), sodium dodecyl benzene sulfonate (SDBS), and any combination thereof.

In some embodiments, the at least one non-ionic surfactant and the at least one anionic surfactant are present in a ratio such that when the at least one non-ionic surfactant and the at least one anionic surfactant are dissolved in a volume of water, the anionic surfactant is present at a concentration of about 0.5-20% (v/v), and the non-ionic surfactant is present at a concentration of about 0.5-20% (v/v). In some embodiments, the anionic surfactant is present at a concentration of about 0.5-20% (w/v), and the non-ionic surfactant is present at a concentration of about 0.5-20% (w/v).

In some embodiments, the at least one non-ionic surfactant and the at least one anionic surfactant are present in the composition in a volume ratio of about 1:100 to 100:1. In some embodiments, the at least one non-ionic surfactant and the at least one anionic surfactant are present in the composition in a volume ratio of about 1:3 to 100:1. In some embodiments, the at least one non-ionic surfactant and the at least one anionic surfactant are present in the composition in a volume ratio of about 1:1 to 100:1. In some embodiments, the at least one non-ionic surfactant and the at least one anionic surfactant are present in the composition in a volume ratio of about 3:1 to 100:1. In some embodiments, the at least one non-ionic surfactant and the at least one anionic surfactant are present in the composition in a volume ratio of about 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1.

In some embodiments, the at least one non-ionic surfactant and the at least one anionic surfactant are present in the composition in a weight ratio of about 1:100 to 100:1. In some embodiments, the at least one non-ionic surfactant and the at least one anionic surfactant are present in the composition in a weight ratio of about 1:3 to 100:1. In some embodiments, the at least one non-ionic surfactant and the at least one anionic surfactant are present in the composition in a weight ratio of about 1:1 to 100:1. In some embodiments, the at least one non-ionic surfactant and the at least one anionic surfactant are present in the composition in a weight ratio of about 3:1 to 100:1. In some embodiments, the at least one non-ionic surfactant and the at least one anionic surfactant are present in the composition in a weight ratio of about 1:100, 1:99, 1:98, 1:97, 1:96, 1:95, 1:94, 1:93, 1:92, 1:91, 1:90, 1:89, 1:88, 1:87, 1:86, 1:85, 1:84, 1:83, 1:82, 1:81, 1:80, 1:79, 1:78, 1:77, 1:76, 1:75, 1:74, 1:73, 1:72, 1:71, 1:70, 1:69, 1:68, 1:67, 1:66, 1:65, 1:64, 1:63, 1:62, 1:61, 1:60, 1:59, 1:58, 1:57, 1:56, 1:55, 1:54, 1:53, 1:52, 1:51, 1:50, 1:49, 1:48, 1:47, 1:46, 1:45, 1:44, 1:43, 1:42, 1:41, 1:40, 1:39, 1:38, 1:37, 1:36, 1:35, 1:34, 1:33, 1:32, 1:31, 1:30, 1:29, 1:28, 1:27, 1:26, 1:25, 1:24, 1:23, 1:22, 1:21, 1:20, 1:19, 1:18, 1:17, 1:16, 1:15, 1:14, 1:13, 1:12, 1:11, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1.

In some embodiments, when the at least one non-ionic surfactant and the at least one anionic surfactant are dissolved in a volume of water, the anionic surfactant is present at a concentration of about 0.03M to 0.4M, and the non-ionic surfactant is present at a concentration of about 1-20% (v/v). In some embodiments, the anionic surfactant is present at a concentration of about 0.05M to 0.15M, and the non-ionic surfactant is present at a concentration of about 5-20% (v/v).

In some embodiments, the anionic surfactant is present at a concentration of about 0.09M to 0.095M, and the non-ionic surfactant is present at a concentration of about 10% (v/v).

In some embodiments, the at least one anionic surfactant is NLS, and the at least one non-ionic surfactant is selected from the group consisting of: (i) the compound of Formula I, wherein n is 7 or 8 or a mixture thereof, and q is 8; or n is 9 or 10 or a mixture thereof, and q is 8; the compound of Formula II, wherein w+x+y+z=20; and (ii) the compound of Formula III, wherein n is 23.

In some embodiments, the at least one anionic surfactant is SDS, and the at least one non-ionic surfactant is selected from the group consisting of: (i) the compound of Formula I, wherein n is 7 or 8 or a mixture thereof, and q is 8; or n is 9 or 10 or a mixture thereof, and q is 8; (ii) the compound of Formula II, wherein w+x+y+z=20; and (iii) the compound of Formula III, wherein n is 23.

In some embodiments, the at least one anionic surfactant is SDBS, and the at least one non-ionic surfactant is selected from the group consisting of: (i) the compound of Formula I, wherein n is 7 or 8 or a mixture thereof, and q is 8; or n is 9 or 10 or a mixture thereof, and q is 8; (ii) the compound of Formula II, wherein w+x+y+z=20; and (iii) the compound of Formula III, wherein n is 23.

In some embodiments, the at least one anionic surfactant is sodium cholate, and the at least one non-ionic surfactant is selected from the group consisting of: (i) the compound of Formula I, wherein n is 9 or 10 or a mixture thereof, and q is 8; (ii) the compound of Formula II, wherein w+x+y+z=20; and (iii) the compound of Formula III, wherein n is 23.

In some embodiments, the at least one anionic surfactant is NLS, and the at least one non-ionic surfactant is Formula I, wherein n is 9 or 10 or a mixture thereof, and q is 8, and the average molecular weight of the at least one non-ionic surfactant is about 625 g/mol.

In some embodiments, the at least one anionic surfactant is NLS, and the at least one non-ionic surfactant is Formula I, wherein n is 9 or 10 or a mixture thereof, and q is 8, and the average molecular weight of the at least one non-ionic surfactant is about 603 g/mol.

In some embodiments, the at least one anionic surfactant is NLS, and the at least one non-ionic surfactant is Formula I, wherein n is 7 or 8 or a mixture thereof, and q is 8.

In some embodiments, the at least one anionic surfactant is NLS, and the at least one non-ionic surfactant is Formula III, wherein n is 23.

In some embodiments, the at least one anionic surfactant is NLS, and the at least one non-ionic surfactant is Formula II, wherein w+x+y+z=20.

In some embodiments, the at least one anionic surfactant is SDS, and the at least one non-ionic surfactant is Formula I, wherein n is 9 or 10 or a mixture thereof, and q is 8, and the average molecular weight of the at least one non-ionic surfactant is about 625 g/mol.

In some embodiments, the at least one anionic surfactant is SDS, and the at least one non-ionic surfactant is Formula I, wherein n is 9 or 10 or a mixture thereof, and q is 8, and the average molecular weight of the at least one non-ionic surfactant is about 603 g/mol.

In some embodiments, the at least one anionic surfactant is SDS, and the at least one non-ionic surfactant is Formula I, wherein n is 7 or 8 or a mixture thereof, and q is 8.

In some embodiments, the at least one anionic surfactant is SDS, and the at least one non-ionic surfactant is Formula III, wherein n is 23.

In some embodiments, the at least one anionic surfactant is SDS, and the at least one non-ionic surfactant is Formula II, wherein w+x+y+z=20.

In some embodiments, the at least one anionic surfactant is SDBS, the at least one non-ionic surfactant is Formula I, wherein n is 9 or 10 or a mixture thereof, and q is 8, and the average molecular weight of the at least one non-ionic surfactant is about 625 g/mol.

In some embodiments, the at least one anionic surfactant is SDBS, the at least one non-ionic surfactant is Formula I, wherein n is 9 or 10 or a mixture thereof, and q is 8, and the average molecular weight of the at least one non-ionic surfactant is about 603 g/mol.

In some embodiments, the at least one anionic surfactant is SDBS, and the at least one non-ionic surfactant is Formula I, wherein n is 7 or 8 or a mixture thereof, and q is 8.

In some embodiments, the at least one anionic surfactant is SDBS, and the at least one non-ionic surfactant is Formula III, wherein n is 23.

In some embodiments, the at least one anionic surfactant is SDBS, and the at least one non-ionic surfactant is Formula II, wherein w+x+y+z=20.

In some embodiments, the at least one anionic surfactant is sodium cholate, the at least one non-ionic surfactant is Formula I, wherein n is 9 or 10 or a mixture thereof, and q is 8, and the average molecular weight of the at least one non-ionic surfactant is about 625 g/mol.

In some embodiments, the at least one anionic surfactant is sodium cholate, the at least one non-ionic surfactant is Formula I, wherein n is 9 or 10 or a mixture thereof, and q is 8, and the average molecular weight of the at least one non-ionic surfactant is about 603 g/mol.

In some embodiments, the at least one anionic surfactant is sodium cholate, and the at least one non-ionic surfactant is Formula I, wherein n is 7 or 8 or a mixture thereof, and q is 8.

In some embodiments, the at least one anionic surfactant is sodium cholate, and the at least one non-ionic surfactant is Formula III, wherein n is 23.

In some embodiments, the at least one anionic surfactant is sodium cholate, and the at least one non-ionic surfactant is Formula II, wherein w+x+y+z=20.

In some embodiments, the composition further includes one or more of a pH buffer, salt, proteinase, or combination thereof.

In some embodiments, the pH buffer is Tris-HCl.

In some embodiments, the pH buffer has a concentration of about 0.3-3M. In some embodiments, the pH buffer has a concentration of about 1.5-3M.

In some embodiments, the salt is selected from the group consisting of NaCl, KCl, NH₄Cl, Na₂SO₄, K₂SO₄, (NH₄)₂SO₄, guanidinium hydrochloride, and guanidinium thiocyanate.

In some embodiments, the salt is NaCl.

In some embodiments, the salt has a concentration of about 0.3-3M. In some embodiments, the salt has a concentration of about 1-2M.

In some embodiments, the proteinase is selected from the group consisting of Arg-C proteinase, BNPS-Skatole, Caspase3, Caspase6, Caspase9, Chymotrypsin-high specificity (C-term to [FYW], not before P), Clostripain (Clostridiopeptidase B), Factor Xa, GranzymeB, LysC, Neutrophil elastase, Pepsin (pH1.3), Proteinase K, Thermolysin, Asp-N endopeptidase, Caspase1, Caspase4, Caspase7, Caspase10, Chymotrypsin-low specificity (C-term to [FYWML], not before P), CNBr, Formic acid, Hydroxylamine, LysN, Pepsin, Staphylococcal peptidase I, Thrombin, Asp-N endopeptidase+N-terminal Glu, Caspase2, Caspase5, Caspase8, Enterokinase, Glutamyl endopeptidase, Iodosobenzoic acid, 2-nitro-5-thiocyanobenzoic acid (NTCB), Proline-endopeptidase, Tobacco etch virus protease, and Trypsin.

In some embodiments, the proteinase is Proteinase K.

In some embodiments, the composition further includes NaCl present at a concentration of about 1.35M and Tris-HCl present at a concentration of about 2.25M.

In some embodiments, the at least one anionic surfactant is 0.88-11.7% (w/v) NLS; the at least one non-ionic surfactant is 0.94-15.9% (w/v) Triton X-100, 0.94-15.9% (w/v) Triton X-114, 0.97-15.45% (w/v) Triton X-45, 0.97-15.45% (w/v) Brij L23, 0.91-16.43% (w/v) Tween 20, or 0.94-15.90% (w/v) IGEPAL CA630; and wherein the composition further includes 0.3-3M NaCl and 0.3-3M Tris-HCl.

In some embodiments, the at least one anionic surfactant is 0.86-11.52% (w/v) SDS; and the at least one non-ionic surfactant is 0.94-15.9% (w/v) Triton X-100, 0.94-15.9% (w/v) Triton X-114, 0.97-15.45% (w/v) Triton X-45, 0.97-15.45% (w/v) Brij L23, 0.91-16.43% (w/v) Tween 20, or 0.94-15.90% (w/v) IGEPAL CA630.

In some embodiments, the at least one anionic surfactant is 1.04-13.9% (w/v) SDBS; and the at least one non-ionic surfactant is 0.94-15.9% (w/v) Triton X-100, 0.94-15.9% (w/v) Triton X-114, 0.97-15.45% (w/v) Triton X-45, 0.97-15.45% (w/v) Brij L23, 0.91-16.43% (w/v) Tween 20, or 0.94-15.90% (w/v) IGEPAL CA630.

In some embodiments, the at least one anionic surfactant is 1.29-17.24% (w/v) sodium cholate; and the at least one non-ionic surfactant is 0.94-15.9% (w/v) Triton X-100, 0.94-15.9% (w/v) Triton X-114, 0.97-15.45% (w/v) Triton X-45, 0.97-15.45% (w/v) Brij L23, 0.91-16.43% (w/v) Tween 20, or 0.94-15.90% (w/v) IGEPAL CA630.

In some embodiments, the at least one anionic surfactant is 0.07-0.11M NLS, the at least one non-ionic surfactant is 8-12% (v/v) Formula I, wherein n is 9 or 10 or a mixture thereof, and q is 8, the average molecular weight of the at least one non-ionic surfactant is about 625 g/mol, and wherein the composition further includes 1.2-1.5M NaCl and 2.1-2.4M Tris-HCl.

In some embodiments, the at least one anionic surfactant is 0.07-0.11M NLS, the at least one non-ionic surfactant is 8-12% (v/v) Formula I, wherein n is 9 or 10 or a mixture thereof, and q is 8, the average molecular weight of the at least one non-ionic surfactant is about 603 g/mol, and wherein the composition further includes 1.2-1.5M NaCl and 2.1-2.4M Tris-HCl.

In some embodiments, the at least one anionic surfactant is 0.07-0.11M NLS, the at least one non-ionic surfactant is 8-12% (v/v) Formula I, wherein n is 7 or 8 or a mixture thereof, and q is 8, and wherein the composition further includes 1.2-1.5M NaCl and 2.1-2.4M Tris-HCl.

In some embodiments, the at least one anionic surfactant is 0.07-0.11M NLS, the at least one non-ionic surfactant is 8-12% (v/v) Formula III, wherein n is 23, and wherein the composition further includes 1.2-1.5M NaCl and 2.1-2.4M Tris-HCl.

In some embodiments, the at least one anionic surfactant is 0.07-0.11M NLS, the at least one non-ionic surfactant is 8-12% (v/v) Formula II, wherein w+x+y+z is 20, and wherein the composition further includes 1.2-1.5M NaCl and 2.1-2.4M Tris-HCl.

In some embodiments, the at least one anionic surfactant is 0.07-0.11M SDS, the at least one non-ionic surfactant is 8-12% (v/v) Formula I, wherein n is 9 or 10 or a mixture thereof, and q is 8, and the average molecular weight of the at least one non-ionic surfactant is about 625 g/mol.

In some embodiments, the at least one anionic surfactant is 0.07-0.11M SDS, the at least one non-ionic surfactant is 8-12% (v/v) Formula I, wherein n is 9 or 10 or a mixture thereof, and q is 8, and the average molecular weight of the at least one non-ionic surfactant is about 603 g/mol.

In some embodiments, the at least one anionic surfactant is 0.07-0.11M SDS, and the at least one non-ionic surfactant is 8-12% (v/v) Formula I, wherein n is 7 or 8 or a mixture thereof, and q is 8.

In some embodiments, the at least one anionic surfactant is 0.07-0.11M SDS, and the at least one non-ionic surfactant is 8-12% (v/v) Formula III, wherein n is 23.

In some embodiments, the at least one anionic surfactant is 0.07-0.11M SDS, and the at least one non-ionic surfactant is 8-12% (v/v) Formula II, wherein w+x+y+z is 20.

In some embodiments, the at least one anionic surfactant is 0.07-0.11M SDS, and the at least one non-ionic surfactant is 8-12% (v/v) Formula I, wherein n is 9 or 10 or a mixture thereof, and q is 8.

In some embodiments, the at least one anionic surfactant is 0.07-0.11M SDBS, the at least one non-ionic surfactant is 8-12% (v/v) Formula I, wherein n is 9 or 10 or a mixture thereof, and q is 8, and the average molecular weight of the at least one non-ionic surfactant is about 625 g/mol.

In some embodiments, the at least one anionic surfactant is 0.07-0.11M SDBS, the at least one non-ionic surfactant is 8-12% (v/v) Formula I, wherein n is 9 or 10 or a mixture thereof, and q is 8, and the average molecular weight of the at least one non-ionic surfactant is about 603 g/mol.

In some embodiments, the at least one anionic surfactant is 0.07-0.11M SDBS, and the at least one non-ionic surfactant is 8-12% (v/v) Formula I, wherein n is 7 or 8 or a mixture thereof, and q is 8.

In some embodiments, the at least one anionic surfactant is 0.07-0.11M SDBS, and the at least one non-ionic surfactant is 8-12% (v/v) Formula III, wherein n is 23.

In some embodiments, the at least one anionic surfactant is 0.07-0.11M SDBS, and the at least one non-ionic surfactant is 8-12% (v/v) Formula II, wherein w+x+y+z is 20.

In some embodiments, the at least one anionic surfactant is 0.07-0.11M SDBS, and the at least one non-ionic surfactant is 8-12% (v/v) Formula I, wherein n is 9 or 10 or a mixture thereof, and q is 8.

In some embodiments, the at least one anionic surfactant is 0.07-0.11M sodium cholate, the at least one non-ionic surfactant is 8-12% (v/v) Formula I, wherein n is 9 or 10 or a mixture thereof, and q is 8, and the average molecular weight of the at least one non-ionic surfactant is about 625 g/mol.

In some embodiments, the at least one anionic surfactant is 0.07-0.11M sodium cholate, the at least one non-ionic surfactant is 8-12% (v/v) Formula I, wherein n is 9 or 10 or a mixture thereof, and q is 8, and the average molecular weight of the at least one non-ionic surfactant is about 603 g/mol.

In some embodiments, the at least one anionic surfactant is 0.07-0.11M sodium cholate, and the at least one non-ionic surfactant is 8-12% (v/v) Formula I, wherein n is 7 or 8 or a mixture thereof, and q is 8.

In some embodiments, the at least one anionic surfactant is 0.07-0.11M sodium cholate, and the at least one non-ionic surfactant is 8-12% (v/v) Formula III, wherein n is 23.

In some embodiments, the at least one anionic surfactant is 0.07-0.11M sodium cholate, and the at least one non-ionic surfactant is 8-12% (v/v) Formula II, wherein w+x+y+z is 20.

In some embodiments, the at least one anionic surfactant is 0.07-0.11M sodium cholate, and the at least one non-ionic surfactant is 8-12% (v/v) Formula I, wherein n is 9 or 10 or a mixture thereof, and q is 8.

In some embodiments, the at least one anionic surfactant is about 0.095M NLS, the at least one non-ionic surfactant is about 10% (v/v) Formula I, wherein n is 9 or 10 or a mixture thereof, and q is 8, the average molecular weight of the at least one non-ionic surfactant is about 625 g/mol, and wherein the composition further includes about 1.35 M NaCl and about 2.25 M Tris-HCl.

In some embodiments, the at least one anionic surfactant is about 0.095M NLS, the at least one non-ionic surfactant is about 10% (v/v) Formula I, wherein n is 9 or 10 or a mixture thereof, and q is 8, the average molecular weight of the at least one non-ionic surfactant is about 603 g/mol, and wherein the composition further includes about 1.35 M NaCl and about 2.25 M Tris-HCl.

In some embodiments, the at least one anionic surfactant is about 0.095M NLS, the at least one non-ionic surfactant is about 10% (v/v) Formula I, wherein n is 7 or 8 or a mixture thereof, and q is 8, and wherein the composition further includes 1 about 1.35 M NaCl and about 2.25 M Tris-HCl.

In some embodiments, the at least one anionic surfactant is about 0.095M NLS, the at least one non-ionic surfactant is about 10% (v/v) Formula III, wherein n is 23, and wherein the composition further about 1.35 M NaCl and about 2.25 M Tris-HCl.

In some embodiments, the at least one anionic surfactant is about 0.095M NLS, the at least one non-ionic surfactant is about 10% (v/v) Formula II, wherein w+x+y+Z is 20, and wherein the composition further includes about 1.35 M NaCl and about 2.25 M Tris-HCl.

In some embodiments, the at least one anionic surfactant is about 0.09M SDS, the at least one non-ionic surfactant is about 10% (v/v) Formula I, wherein n is 9 or 10 or a mixture thereof, and q is 8, and the average molecular weight of the at least one non-ionic surfactant is about 625 g/mol.

In some embodiments, the at least one anionic surfactant is about 0.09M SDS, the at least one non-ionic surfactant is about 10% (v/v) Formula I, wherein n is 9 or 10 or a mixture thereof, and q is 8, and the average molecular weight of the at least one non-ionic surfactant is about 603 g/mol.

In some embodiments, the at least one anionic surfactant is about 0.09M SDS, and the at least one non-ionic surfactant is about 10% (v/v) Formula I, wherein n is 7 or 8 or a mixture thereof, and q is 8.

In some embodiments, the at least one anionic surfactant is about 0.09M SDS, and the at least one non-ionic surfactant is about 10% (v/v) Formula III, wherein n is 23.

In some embodiments, the at least one anionic surfactant is about 0.09M SDS, and the at least one non-ionic surfactant is about 10% (v/v) Formula II, wherein w+x+y+z is 20.

In some embodiments, the at least one anionic surfactant is about 0.09M SDS, and the at least one non-ionic surfactant is about 10% (v/v) Formula I, wherein n is 9 or 10 or a mixture thereof, and q is 8.

In some embodiments, the at least one anionic surfactant is about 0.09M SDBS, the at least one non-ionic surfactant is about 10% (v/v) Formula I, wherein n is 9 or 10 or a mixture thereof, and q is 8, and the average molecular weight of the at least one non-ionic surfactant is about 625 g/mol.

In some embodiments, the at least one anionic surfactant is about 0.09M SDBS, the at least one non-ionic surfactant is about 10% (v/v) Formula I, wherein n is 9 or 10 or a mixture thereof, and q is 8, and the average molecular weight of the at least one non-ionic surfactant is about 603 g/mol.

In some embodiments, the at least one anionic surfactant is about 0.09M SDBS, and the at least one non-ionic surfactant is about 10% (v/v) Formula I, wherein n is 7 or 8 or a mixture thereof, and q is 8.

In some embodiments, the at least one anionic surfactant is about 0.09M SDBS, and the at least one non-ionic surfactant is about 10% (v/v) Formula III, wherein n is 23.

In some embodiments, the at least one anionic surfactant is about 0.09M SDBS, and the at least one non-ionic surfactant is about 10% (v/v) Formula II, wherein w+x+y+z is 20.

In some embodiments, the at least one anionic surfactant is about 0.09M SDBS, and the at least one non-ionic surfactant is about 10% (v/v) Formula I, wherein n is 9 or 10 or a mixture thereof, and q is 8.

In some embodiments, the at least one anionic surfactant is about 0.09M sodium cholate, the at least one non-ionic surfactant is about 10% (v/v) Formula I, wherein n is 9 or 10 or a mixture thereof, and q is 8, and the average molecular weight of the at least one non-ionic surfactant is about 625 g/mol.

In some embodiments, the at least one anionic surfactant is about 0.09M sodium cholate, the at least one non-ionic surfactant is about 10% (v/v) Formula I, wherein n is 9 or 10 or a mixture thereof, and q is 8, and the average molecular weight of the at least one non-ionic surfactant is about 603 g/mol.

In some embodiments, the at least one anionic surfactant is about 0.09M sodium cholate, and the at least one non-ionic surfactant is about 10% (v/v) Formula I, wherein n is 7 or 8 or a mixture thereof, and q is 8.

In some embodiments, the at least one anionic surfactant is about 0.09M sodium cholate, and the at least one non-ionic surfactant is about 10% (v/v) Formula III, wherein n is 23.

In some embodiments, the at least one anionic surfactant is about 0.09M sodium cholate, and the at least one non-ionic surfactant is about 10% (v/v) Formula II, wherein w+x+y+z is 20.

In some embodiments, the at least one anionic surfactant is about 0.09M sodium cholate, and the at least one non-ionic surfactant is about 10% (v/v) Formula I, wherein n is 9 or 10 or a mixture thereof, and q is 8.

In some embodiments, provided is a kit including: (a) the lysing composition of any one of the embodiments as described herein; and (b) at least one aqueous two-phase system (ATPS) composition, wherein the at least one aqueous two-phase system (ATPS) includes a polymer, a salt, a surfactant, or any combination thereof.

In one embodiment, the concentration of the anionic surfactant in the composition is in the range of about 0.03M to 0.4M. In one embodiment, the concentration of the anionic surfactant in the composition is in the range of about 0.05M to 0.35M, 0.05M to 0.3M, 0.05M to 0.25M, 0.05M to 0.2M, or 0.05M to 0.15M. In one embodiment, the concentration of the anionic surfactant in the composition is in the range of about 0.06M to 0.12M. In one embodiment, the concentration of the anionic surfactant in the composition is in the range of about 0.07M to 0.11M. In one embodiment, the concentration of the anionic surfactant in the composition is in the range of about 0.08M to 0.10M. In various embodiments, the concentration of the anionic surfactant in the composition is about 0.05M, about 0.055M, about 0.06M, about 0.065M, about 0.07M, about 0.075M, about 0.08M, about 0.085M, about 0.09M, about 0.095M, about 0.1M, about 0.105M, about 0.11M, about 0.115M, about 0.12M, about 0.125M, about 0.13M, about 0.135M, about 0.14M, about 0.145M, or about 0.15M.

In one embodiment, the concentration of the anionic surfactant in the composition is in the range of about 0.5-20% (v/v). In various embodiments, the concentration of the anionic surfactant in the composition is about 0.5% (v/v), about 1% (v/v), about 1.5% (v/v), about 2% (v/v), about 2.5% (v/v), about 3% (v/v), about 3.5% (v/v), about 4% (v/v), about 4.5% (v/v), about 5% (v/v), about 5.5% (v/v), about 6% (v/v), about 6.5% (v/v), about 7% (v/v), about 7.5% (v/v), about 8% (v/v), about 8.5% (v/v), about 9% (v/v), about 9.5% (v/v), about 10% (v/v), about 10.5% (v/v), about 11% (v/v), about 11.5% (v/v), about 12% (v/v), about 12.5% (v/v), about 13% (v/v), about 13.5% (v/v), about 14% (v/v), about 14.5% (v/v), about 15% (v/v), about 15.5% (v/v), about 16% (v/v), about 16.5% (v/v), about 17% (v/v), about 17.5% (v/v), about 18% (v/v), about 18.5% (v/v), about 19% (v/v), about 19.5% (v/v), or about 20% (v/v).

In one embodiment, the concentration of the non-ionic surfactant in the composition is in the range of about 0.5-20% (v/v). In one embodiment, the concentration of the non-ionic surfactant in the composition is in the range of about 1-20% (v/v). In one embodiment, the concentration of the non-ionic surfactant in the composition is in the range of about 1-10% (v/v), 1-15% (v/v), 2-20% (v/v), 5-15% (v/v), 7-15% (v/v), or 8-12% (v/v). In various embodiments, the concentration of the anionic surfactant in the composition is 0.5% (v/v), about 1% (v/v), about 1.5% (v/v), about 2% (v/v), about 2.5% (v/v), about 3% (v/v), about 3.5% (v/v), about 4% (v/v), about 4.5% (v/v), about 5% (v/v), about 5.5% (v/v), about 6% (v/v), about 6.5% (v/v), about 7% (v/v), about 7.5% (v/v), about 8% (v/v), about 8.5% (v/v), about 9% (v/v), about 9.5% (v/v), about 10% (v/v), about 10.5% (v/v), about 11% (v/v), about 11.5% (v/v), about 12% (v/v), about 12.5% (v/v), about 13% (v/v), about 13.5% (v/v), about 14% (v/v), about 14.5% (v/v), about 15% (v/v), about 15.5% (v/v), about 16% (v/v), about 16.5% (v/v), about 17% (v/v), about 17.5% (v/v), about 18% (v/v), about 18.5% (v/v), about 19% (v/v), about 19.5% (v/v), or about 20% (v/v).

In one embodiment, the concentration of the anionic surfactant in the composition is in the range of about 0.5-20% (w/v). In various embodiments, the concentration of the anionic surfactant in the composition is about 0.5% (w/v), about 1% (w/v), about 1.5% (w/v), about 2% (w/v), about 2.5% (w/v), about 3% (w/v), about 3.5% w/v), about 4% (w/v), about 4.5% (w/v), about 5% (w/v), about 5.5% (w/v), about 6% (w/v), about 6.5% (w/v), about 7% (w/v), about 7.5% (w/v), about 8% (w/v), about 8.5% (w/v), about 9% (w/v), about 9.5% (w/v), about 10% (w/v), about 10.5% (w/v), about 11% (w/v), about 11.5% (w/v), about 12% (w/v), about 12.5% (w/v), about 13% (w/v), about 13.5% (w/v), about 14% (w/v), about 14.5% (w/v), about 15% (w/v), about 15.5% (w/v), about 16% (w/v), about 16.5% (w/v), about 17% (w/v), about 17.5% (w/v), about 18% (w/v), about 18.5% (w/v), about 19% (w/v), about 19.5% (w/v), or about 20% (w/v).

In one embodiment, the concentration of the non-ionic surfactant in the composition is in the range of about 0.5-20% (w/v). In one embodiment, the concentration of the non-ionic surfactant in the composition is in the range of about 1-20% (w/v). In one embodiment, the concentration of the non-ionic surfactant in the composition is in the range of about 1-10% (w/v), 1-15% (w/v), 2-20% (w/v), 5-15% (w/v), 7-15% (w/v), or 8-12% (w/v). In various embodiments, the concentration of the anionic surfactant in the composition is 0.5% (w/v), about 1% (w/v), about 1.5% (w/v), about 2% (w/v), about 2.5% (w/v), about 3% (w/v), about 3.5% (w/v), about 4% (w/v), about 4.5% (w/v), about 5% (w/v), about 5.5% (w/v), about 6% (w/v), about 6.5% (w/v), about 7% (w/v), about 7.5% (w/v), about 8% (w/v), about 8.5% (w/v), about 9% (w/v), about 9.5% (w/v), about 10% (w/v), about 10.5% (w/v), about 11% (w/v), about 11.5% (w/v), about 12% (w/v), about 12.5% (w/v), about 13% (w/v), about 13.5% (w/v), about 14% (w/v), about 14.5% (w/v), about 15% (w/v), about 15.5% (w/v), about 16% (w/v), about 16.5% (w/v), about 17% (w/v), about 17.5% (w/v), about 18% (w/v), about 18.5% (w/v), about 19% (w/v), about 19.5% (w/v), or about 20% (w/v).

In some embodiments, the at least one non-ionic surfactant has a hydrophilic-lipophilic balance (HLB) value greater than or equal to 10. In some embodiments, the hydrophilic-lipophilic balance (HLB) value of the at least one non-ionic surfactant is between 10 and 20.

In various embodiments, the HLB value of the at least one non-ionic surfactant is about 10, about 10.1, about 10.2, about 10.1, about 10.2, about 10.3, about 10.4, about 10.5, about 10.6, about 10.7, about 10.8, about 10.9, about 11, about 11.1, about 11.2, about 11.3, about 11.4, about 11.5, about 11.6, about 11.7, about 11.8, about 11.9, about 12, about 12.1, about 12.2, about 12.3, about 12.4, about 12.5, about 12.6, about 12.7, about 12.8, about 12.9, about 13, about 13.1, about 13.2, about 13.3, about 13.4, about 13.5, about 13.6, about 13.7, about 13.8, about 13.9, about 14, about 14.1, about 14.2, about 14.3, about 14.4, about 14.5, about 14.6, about 14.7, about 14.8, about 14.9, about 15, about 15.1, about 15.2, about 15.3, about 15.4, about 15.5, about 15.6, about 15.7, about 15.8, about 15.9, about 16, about 16.1, about 16.2, about 16.3, about 16.4, about 16.5, about 16.6, about 16.7, about 16.8, about 16.9, about 17, about 17.1, about 17.2, about 17.3, about 17.4, about 17.5, about 17.6, about 17.7, about 17.8, about 17.9, about 18, about 18.1, about 18.2, about 18.3, about 18.4, about 18.5, about 18.6, about 18.7, about 18.8, about 18.9, about 19, about 19.1, about 19.2, about 19.3, about 19.4, about 19.5, about 19.6, about 19.7, about 19.8, about 19.9, or about 20. In some embodiments, the range is between 10 and one of the HLB values recited above.

In some embodiments, the pH buffer has a concentration in the range of about 0.3-3M, about 0.5-3M, about 1-3M, about 1.5-3M, about 1-4M, or about 2-2.5M.

In some embodiments, the pH buffer has a concentration in the range of about 0.5-2.5M, about 1-2M, or about 0.75-1.5M.

Various ATPS systems that can be used in various embodiments of the present invention include, but are not limited to, polymer-polymer, polymer-salt, polymer-surfactant, salt-surfactant, surfactant, surfactant-surfactant, or polymer-salt-surfactant.

In one embodiment, the first and/or second ATPS composition comprises a polymer. In some embodiments, polymers that may be employed include, but are not limited to, polyalkylene glycols, such as hydrophobically modified polyalkylene glycols, poly(oxyalkylene) polymers, poly(oxyalkylene) copolymers, such as hydrophobically modified poly(oxyalkylene) copolymers, polyvinyl pyrrolidone, polyvinyl alcohol, polyvinyl caprolactam, polyvinyl methylether, alkoxylated surfactants, alkoxylated starches, alkoxylated cellulose, alkyl hydroxyalkyl cellulose, silicone-modified polyethers, and poly N-isopropylacrylamide and copolymers thereof. In another embodiment, the first phase forming polymer comprises polyethylene glycol (PEG), polypropylene glycol (PPG), or dextran. In some embodiments, the polymer is selected from the group consisting of polyether, polyimine, polyalkylene glycol, vinyl polymer, alkoxylated surfactant, polysaccharides, alkoxylated starch, alkoxylated cellulose, alkyl hydroxyalkyl cellulose, polyether-modified silicones, polyacrylamide, polyacrylic acid and copolymer thereof. In some embodiments, the polymer is selected from the group consisting of dipropylene glycol, tripropylene glycol, polyethylene glycol, polypropylene glycol, poly(ethylene glycol-propylene glycol), poly(ethylene glycol-ran-propylene glycol), polyvinyl pyrrolidone, polyvinyl alcohol, polyvinyl caprolactam, polyvinyl methylether, dextran, carboxymethyl dextran, dextran sulfate, hydroxypropyl dextran, starch, carboxymethyl cellulose, polyacrylic acid, hydroxypropyl cellulose, methyl cellulose, ethylhydroxyethylcellulose, maltodextrin, polyethyleneimine, poly N-isopropylacrylamide and copolymers thereof. In some embodiments, the polymer is selected from the group consisting of dipropylene glycol, tripropylene glycol, polyethylene glycol, polypropylene glycol, poly(ethylene glycol-propylene glycol), poly(ethylene glycol-ran-propylene glycol), polyvinyl pyrrolidone, polyvinyl alcohol, polyvinyl caprolactam, polyvinyl methylether and poly N-isopropylacrylamide. In some embodiments, the polymer is selected from the group consisting of polyacrylamide, polyacrylic acid and copolymers thereof. In some embodiments, the polymer is selected from the group consisting of dextran, carboxymethyl dextran, dextran sulfate, hydroxypropyl dextran and starch. In some embodiments, the polymer has an average molecular weight in the range of 200-1,000 Da, 200-35,000 Da, 425-2,000 Da, 400-35,000 Da, 980-12,000 Da, or 3,400-5,000,000 Da. In some embodiments, the polymer comprises ethylene oxide and propylene oxide units, and the polymer has an EO:PO ratio of 90:10 to 10:90.

In one embodiment, the polymer concentration of the first and/or second ATPS composition is in the range of about 4% to about 84% by weight of the total weight of the aqueous solution (w/w). In one embodiment, the polymer concentration of the first and/or second ATPS composition is in the range of about 5-80% (w/w), about 10-60% (w/w), about 15-45% (w/w), about 20-30% (w/w), about 30-50% (w/w), or about 50-80% (w/w). In various embodiments, the polymer solution is selected from a polymer solution that is about 4% w/w, about 4.5% w/w, about 5% w/w, about 5.5% w/w, about 6% w/w, about 6.5% w/w, about 7% w/w, about 7.5% w/w, about 8% w/w, about 8.5% w/w, about 9% w/w, about 9.5% w/w, about 10% w/w, about 10.5% w/w, about 11% w/w, about 11.5% w/w, about 12% w/w, about 12.5% w/w, about 13% w/w, about 13.5% w/w, about 14% w/w, about 14.5% w/w, about 15% w/w, about 15.5% w/w, about 16% w/w, about 16.5% w/w, about 17% w/w, about 17.5% w/w, about 18% w/w, about 18.5% w/w, about 19% w/w, about 19.5% w/w, about 20% w/w, about 20.5% w/w, about 21% w/w, about 21.5% w/w, about 22% w/w, about 22.5% w/w, about 23% w/w, about 23.5% w/w, about 24% w/w, about 24.5% w/w, about 35% w/w, about 35.5% w/w, about 36% w/w, about 36.5% w/w, about 37% w/w, about 37.5% w/w, about 38% w/w, about 38.5% w/w, about 39% w/w, about 39.5% w/w, about 40% w/w, about 40.5% w/w, about 41% w/w, about 41.5% w/w, about 42% w/w, about 42.5% w/w, about 43% w/w, about 43.5% w/w, about 44% w/w, about 44.5% w/w, about 45% w/w, about 45.5% w/w, about 46% w/w, about 46.5% w/w, about 47% w/w, about 47.5% w/w, about 48% w/w, about 48.5% w/w, about 49% w/w, about 49.5% w/w, about 50% w/w, about 50.5% w/w, about 51% w/w, about 51.5% w/w, about 52% w/w, about 52.5% w/w, about 53% w/w, about 53.5% w/w, about 54% w/w, about 54.5% w/w, about 55% w/w, about 55.5% w/w, about 56% w/w, about 56.5% w/w, about 57% w/w, about 57.5% w/w, about 58% w/w, about 58.5% w/w, about 59% w/w, about 59.5% w/w, about 60% w/w, about 60.5% w/w, about 61% w/w, about 61.5% w/w, about 62% w/w, about 62.5% w/w, about 63% w/w, about 63.5% w/w, about 64% w/w, about 64.5% w/w, about 65% w/w, about 65.5% w/w, about 66% w/w, about 66.5% w/w, about 67% w/w, about 67.5% w/w, about 68% w/w, about 68.5% w/w, about 69% w/w, about 69.5% w/w, about 70% w/w, about 70.5% w/w, about 71% w/w, about 71.5% w/w, about 72% w/w, about 72.5% w/w, about 73% w/w, about 73.5% w/w, about 74% w/w, about 74.5% w/w, about 75% w/w, about 75.5% w/w, about 76% w/w, about 76.5% w/w, about 77% w/w, about 77.5% w/w, about 78% w/w, about 78.5% w/w, about 79% w/w, about 79.5% w/w, about 80% w/w, about 80.5% w/w, about 81% w/w, about 81.5% w/w, about 82% w/w, about 82.5% w/w, about 83% w/w, about 83.5% w/w, and about 84% w/w.

In one embodiment, the first and/or second ATPS composition comprises a salt and thereby forms a salt solution. In some embodiments, the salt includes, but is not limited to, kosmotropic salts, chaotropic salts, inorganic salts containing cations such as straight or branched trimethyl ammonium, triethyl ammonium, tripropyl ammonium, tributyl ammonium, tetramethyl ammonium, tetraethyl ammonium, tetrapropyl ammonium and tetrabutyl ammonium, and anions such as phosphates, sulphate, nitrate, chloride and hydrogen carbonate. In another embodiment, the salt comprises NaCl, Na₃PO₄, K₃PO₄, Na₂SO₄, potassium citrate, (NH₄)₂SO₄, sodium citrate, sodium acetate or combinations thereof. Other salts, e.g. ammonium acetate, may also be used. In another embodiment, the salt may be selected from magnesium salt, a lithium salt, a sodium salt, a potassium salt, a cesium salt, a zinc salt and an aluminum salt. In some embodiments, the salt may be selected from a bromide salt, an iodide salt, a fluoride salt, a carbonate salt, a sulfate salt, a citrate salt, a carboxylate salt, a borate salt, and a phosphate salt. In some embodiments, the salt comprises potassium phosphate. In some embodiments, the salt comprises ammonium sulfate.

In one embodiment, the total salt concentration is in the range of about 0.01% to about 90%. A skilled person in the art will understand that the amount of salt needed to form an aqueous two-phase system will be influenced by molecular weight, concentration and physical status of the polymer.

In various embodiments, the salt concentration is about 1%-55% w/w. In various embodiments, the salt concentration is about 1-10% w/w, about 1-20% w/w, about about 1.5-45% w/w, or about 5-30% w/w. In various embodiments, the salt concentration is about 1% w/w, about 1.5% w/w, about 2% w/w, about 2.5% w/w, about 3% w/w, about 3.5% w/w, about 4% w/w, about 4.5% w/w, about 5% w/w, about 5.5% w/w, about 6% w/w, about 6.5% w/w, about 7% w/w, about 7.5% w/w, about 8% w/w, about 8.5% w/w, about 9% w/w, about 9.5% w/w, about 10% w/w, about 10.5% w/w, about 11% w/w, about 11.5% w/w, about 12% w/w, about 12.5% w/w, about 13% w/w, about 13.5% w/w, about 14% w/w, about 14.5% w/w, about 15% w/w, about 15.5% w/w, about 16% w/w, about 16.5% w/w, about 17% w/w, about 17.5% w/w, about 18% w/w, about 18.5% w/w, about 19% w/w, about 19.5% w/w, about 20% w/w, about 20.5% w/w, about 21% w/w, about 21.5% w/w, about 22% w/w, about 22.5% w/w, about 23% w/w, about 23.5% w/w, about 24% w/w, about 24.5% w/w, about 35% w/w, about 35.5% w/w, about 36% w/w, about 36.5% w/w, about 37% w/w, about 37.5% w/w, about 38% w/w, about 38.5% w/w, about 39% w/w, about 39.5% w/w, about 40% w/w, about 40.5% w/w, about 41% w/w, about 41.5% w/w, about 42% w/w, about 42.5% w/w, about 43% w/w, about 43.5% w/w, about 44% w/w, about 44.5% w/w, about 45% w/w, about 45.5% w/w, about 46% w/w, about 46.5% w/w, about 47% w/w, about 47.5% w/w, about 48% w/w, about 48.5% w/w, about 49% w/w, about 49.5% w/w, about 50% w/w, about 50.5% w/w, about 51% w/w, about 51.5% w/w, about 52% w/w, about 52.5% w/w, about 53% w/w, about 53.5% w/w, about 54% w/w, about 54.5% w/w, about 55% w/w, about 55.5% w/w, about 56% w/w, about 56.5% w/w, about 57% w/w, about 57.5% w/w, about 58% w/w, about 58.5% w/w, about 59% w/w, about 59.5% w/w, about 60% w/w, about 60.5% w/w, about 61% w/w, about 61.5% w/w, about 62% w/w, about 62.5% w/w, about 63% w/w, about 63.5% w/w, about 64% w/w, about 64.5% w/w, about 65% w/w, about 65.5% w/w, about 66% w/w, about 66.5% w/w, about 67% w/w, about 67.5% w/w, about 68% w/w, about 68.5% w/w, about 69% w/w, about 69.5% w/w, about 70% w/w, about 70.5% w/w, about 71% w/w, about 71.5% w/w, about 72% w/w, about 72.5% w/w, about 73% w/w, about 73.5% w/w, about 74% w/w, about 74.5% w/w, about 75% w/w, about 75.5% w/w, about 76% w/w, about 76.5% w/w, about 77% w/w, about 77.5% w/w, about 78% w/w, about 78.5% w/w, about 79% w/w, about 79.5% w/w, or about 80% w/w.

In one embodiment, the first and/or second ATPS composition comprises a surfactant. In some embodiments, possible surfactants that may be employed include, but are not limited to, Triton-X, Triton-114, Igepal CA-630 and Nonidet P-40, anionic surfactants, such as carboxylates, sulphonates, petroleum sulphonates, alkylbenzenesulphonates, naphthalenesulphonates, olefin sulphonates, alkyl sulphates, sulphates, sulphated natural oils, sulphated natural fats, sulphated esters, sulphated alkanolamides, sulphated alkylphenols, ethoxylated alkylphenols, nonionic surfactants, such as ethoxylated aliphatic alcohol, polyoxyethylene surfactants, carboxylic esters, polyethylene glycol esters, anhydrosorbitol ester, glycol esters of fatty acids, carboxylic amides, monoalkanolamine condensates, polyoxyethylene fatty acid amides, cationic surfactants, such as quaternary ammonium salts, amines with amide linkages, polyoxyethylene alkyl & alicyclic amines, n,n,n′,n′ tetrakis substituted ethylenediamines, 2-alkyl 1-hydroxethyl 2-imidazolines, and amphoteric surfactants, such as n-coco 3-aminopropionic acid and sodium salt thereof, n-tallow 3-iminodipropionate and disodium salt thereof, n-carboxymethyl n dimethyl n-9 octadecenyl ammonium hydroxide, n-cocoamidethyl n hydroxyethylglycine and sodium salt thereof.

In one embodiment, the surfactant concentration of the first ATPS composition is in the range of about 0.05% w/w to about 10% w/w. In one embodiment, the surfactant concentration of the first ATPS composition is in the range of about 0.05-3% w/w, about 0.1-3% w/w, about 1-6% w/w, or about 2-8% w/w. In various embodiments, the surfactant concentration is about 0.05% w/w, 0.1% w/w, about 0.2% w/w, about 0.3% w/w, about 0.4% w/w, about 0.5% w/w, about 0.6% w/w, about 0.7% w/w, about 0.8% w/w, about 0.9% w/w, about 1% w/w, 1.1% w/w, about 1.2% w/w, about 1.3% w/w, about 1.4% w/w, about 1.5% w/w, about 1.6% w/w, about 1.7% w/w, about 1.8% w/w, about 1.9% w/w, about 2% w/w, about 2.1% w/w, about 2.2% w/w, about 2.3% w/w, about 2.4% w/w, about 2.5% w/w, about 2.6% w/w, about 2.7% w/w, about 2.8% w/w, about 2.9% w/w, about 3% w/w, 3.1% w/w, about 3.2% w/w, about 3.3% w/w, about 3.4% w/w, about 3.5% w/w, about 3.6% w/w, about 3.7% w/w, about 3.8% w/w, about 3.9% w/w, about 4% w/w, about 4.1% w/w, about 4.2% w/w, about 4.3% w/w, about 4.4% w/w, about 4.5% w/w, about 4.6% w/w, about 4.7% w/w, about 4.8% w/w, about 4.9% w/w, about 5% w/w, about 5.1% w/w, about 5.2% w/w, about 5.3% w/w, about 5.4% w/w, about 5.5% w/w, about 5.6% w/w, about 5.7% w/w, about 5.8% w/w, about 5.9% w/w, about 6% w/w, 6.1% w/w, about 6.2% w/w, about 6.3% w/w, about 6.4% w/w, about 6.5% w/w, about 6.6% w/w, about 6.7% w/w, about 6.8% w/w, about 6.9% w/w, about 7% w/w, about 7.1% w/w, about 7.2% w/w, about 7.3% w/w, about 7.4% w/w, about 7.5% w/w, about 7.6% w/w, about 7.7% w/w, about 7.8% w/w, about 7.9% w/w, about 8% w/w, about 8.1% w/w, about 8.2% w/w, about 8.3% w/w, about 8.4% w/w, about 8.5% w/w, about 8.6% w/w, about 8.7% w/w, about 8.8% w/w, about 8.9% w/w, about 9% w/w, 9.1% w/w, about 9.2% w/w, about 9.3% w/w, about 9.4% w/w, about 9.5% w/w, about 9.6% w/w, about 9.7% w/w, about 9.8% w/w, about 9.9% w/w, or about 10% w/w.

In one embodiment, the binding buffer comprises a chaotropic agent. In some embodiments, possible chaotropic agents include, but are not limited to, n-butanol, ethanol, guanidinium chloride, guanidinium thiocyanate, lithium perchlorate, lithium acetate, magnesium chloride, phenol, 2-propanol, sodium dodecyl sulfate, thiourea, and urea.

In one embodiment, the concentration of the chaotropic agent in the binding buffer is in the range of about 0.1 M to 8 M. In one embodiment, the concentration of the chaotropic agent in the binding buffer is in the range of about 0.5 M to about 7.5 M, 1 M to 6 M, about 1.5 M to 5 M, or about 2 M to 4 M. In various embodiments, the concentration of the chaotropic agent is about 0.1 M, about 0.2 M, about 0.3 M, about 0.4 M, about 0.5 M, about 0.6 M, about 0.7 M, about 0.8 M, about 0.9 M, about 1 M, about 1.1 M, about 1.2 M, about 1.3 M, about 1.4 M, about 1.5 M, about 1.6 M, about 1.7 M, about 1.8 M, about 1.9 M, about 2 M, about 2.1 M, about 2.2 M, about 2.3 M, about 2.4 M, about 2.5 M, about 2.6 M, about 2.7 M, about 2.8 M, about 2.9 M, about 3 M, about 3.1 M, about 3.2 M, about 3.3 M, about 3.4 M, about 3.5 M, about 3.6 M, about 3.7 M, about 3.8 M, about 3.9 M, about 4 M, about 4.1 M, about 4.2 M, about 4.3 M, about 4.4 M, about 4.5 M, about 4.6 M, about 4.7 M, about 4.8 M, about 4.9 M, about 5 M, about 5.1 M, about 5.2 M, about 5.3 M, about 5.4 M, about 5.5 M, about 5.6 M, about 5.7 M, about 5.8 M, about 5.9 M, about 6 M, about 6.1 M, about 6.2 M, about 6.3 M, about 6.4 M, about 6.5 M, about 6.6 M, about 6.7 M, about 6.8 M, about 6.9 M, about 7 M, about 7.1 M, about 7.2 M, about 7.3 M, about 7.4 M, about 7.5 M, about 7.6 M, about 7.7 M, about 7.8 M, about 7.9 M, or about 8 M.

In one embodiment, the possible extraction columns that may be employed include, but are not limited to, Epoch life science-EconoSpin Silica Membrane Mini Spin Column—1920-250, HiBinds RNA mini—RNACOL-02, Corbition silica spin column—PC0054, PuroSpin micro silica spin—Luna Nano USP003, Purospin nano silica spin—Lunonano USP002, Qiagen RNEasy minElute, Qiagen minElute—700384 Qiagen GMBH, and Qiagen mini.

### EXAMPLES

Provided herein are examples that describe in more detail certain embodiments of the present disclosure. The examples provided herein are merely for illustrative purposes and are not meant to limit the scope of the invention in any way. All references given below and elsewhere in the present application are hereby included by reference.

### Materials

Reagents used in the examples as described herein include ATPS extraction solutions (e.g., purchased from Phase Scientific), Triton X-100, Triton X-114, Triton X-45, Brij L23, Tween 20, IGEPAL CA630, sodium cholate (also referred to as ‘SC’ herein), sodium N-lauroyl sarcosinate (also referred to as ‘NLS’ herein) (30% in aqueous solution), sodium dodecyl sulfate (also referred to as ‘SDS’ herein), sodium dodecyl benzene sulfonate (also referred to as ‘SDBS’ herein), NaCl, EDTA, Tris-HCl, and Proteinase K purchased from Sigma Aldrich; and 145 bp dsDNA purchased from Integrated DNA Technologies. All other chemicals were of analytical grade.

### Example 1: Use of Different Lysis Buffer Compositions to Lyse Plasma Samples Prior to ATPS

### Plasma Cell Lysis

To investigate the effectiveness of lysis with different lysis buffer compositions (also referred to as ‘lysing compositions’ or ‘lysis buffers’), a range of lysis buffers with different combinations of anionic surfactants and non-ionic surfactants have been tested in this study. Pooled plasma was purchased from Innovative Research (EDTA) or TCS Biosciences (citrate). To digest unwanted protein and cells from the plasma samples, 160 μL of lysis buffer and 60 μL of Proteinase K (28.57 mg/mL) were added into a 2 mL aliquot of EDTA (K2) or citrate (TCS) plasma. After vortex for 15 seconds, each sample was incubated at 60° C. for 15 minutes. The lysate was used in further extraction.

Tables 1A-1B show the anionic surfactants and non-ionic surfactants used in the lysis buffers in this study and their properties. The compositions of the lysis buffers used in this study are summarized in Table 2. 100 fg of 145 bp dsDNA was spiked into the above sample.

TABLE 1A
List of example anionic surfactants and non-ionic surfactants used in the lysis buffers

| | | Average | Hydrophilic- | |
|---|---|---|---|---|
| | | molecular | lipophilic | |
| Brand | | weight | balance | |
| name | Linear Formula | (g/mol) | (HLB) | Structure |
| Triton X-100 | (C₂H₄O)ₙC₁₄H₂₂O, n = 9-10 | 625 | 13.4 | 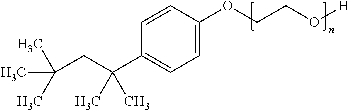 |
| | | | | (Formula I) |
| | | | | n = 9-10 |
| Triton X-114 | (C₂H₄O)ₙC₁₄H₂₂O, n = 7-8 | 537 | 12.4 | 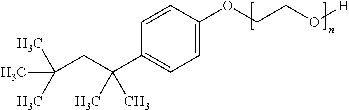 |
| | | | | (Formula I) |
| | | | | n = 7-8 |
| Triton X-45 | (C₂H₄O)ₙC₁₄H₂₂O, n = 4-5 | 383 | 9.8 | 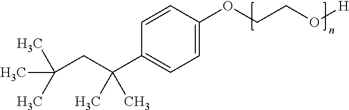 |
| | | | | (Formula I) |
| | | | | n = 4-5 |
| Brij L23 | C₅₈H₁₁₈O₂₄ | ~1198 | 16.9 | CH₃(CH₂)₁₀CH₂(OCH₂CH₂)ₙOH |
| | | | | (Formula III) |
| | | | | n = 23 |
| Tween 20 | C₅H₅((OCH₂CH₂)_{w}OH) ((OCH₂CH₂)ₓOH) (CH(OCH₂CH₂)_{y}OH) CH₂(OCH₂CH₂)_{z}(C═O) CH₂(CH₂)₉CH₃ | ~1228 | 16.7 | 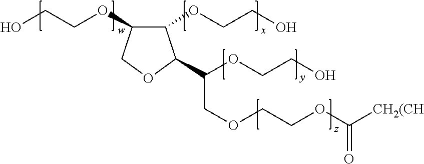 |
| | | | | (Formula II) |
| | | | | w + x + y + z = 20 |
| IGEPA L CA630 | (C₂H₄O)ₙC₁₄H₂₂O, n = 9-10 | 603 | 13.4 | 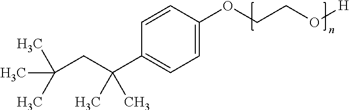 |
| | | | | (Formula I) |
| | | | | n = 9-10 |
| Sodium Cholate (SC) | C₂₄H₃₉NaO₅ | 431 | — | 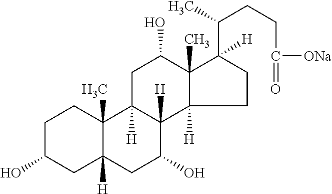 |
| Sodium N- lauroyl sarcosinate (NLS) | CH₃(CH₂)₉CH₂(C═O) NCH₃CH₂(C═O)ONa | 293 | — | 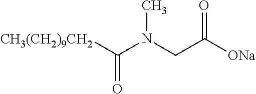 |
| sodium dodecyl sulfate (SDS) | CH₃(CH₂)₁₁OSO₃Na | 288 | — | 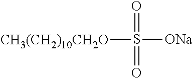 |
| sodium dodecyl benzene sulfonate (SDBS) | CH₃(CH₂)₁₁(C₆H₄)SO₃ Na | 348 | — | 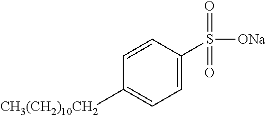 |

TABLE 1B
Properties of example anionic surfactants
and non-ionic surfactants (1 mL = 1 cm³)

| | | | Average | Hydrophilic- |
|---|---|---|---|---|
| | | | molecular | lipophilic |
| | | | weight | balance |
| Brand name | Density | | (g/mol) | (HLB) |
| Triton X-100 | 1.06 | g/mL at 25° C. (lit.) | 625 | 13.4 |
| Triton X-114 | 1.058 | g/mL at 25° C. (lit.) | 537 | 12.4 |
| Triton X-45 | 1.031 | g/mL at 20° C. | 383 | 9.8 |
| Brij L23 | 1.03 | g/cm³ | ~1198 | 16.9 |
| Tween 20 | 1.095 | g/mL at 25° C. (lit.) | ~1228 | 16.7 |
| IGEPAL | 1.06 | g/cm3 at 25° C. (lit.) | 603 | 13.4 |
| CA630 | | | | |

TABLE 2
List of example compositions of lysis buffers

| Lysis | Anionic | Non-ionic | |
|---|---|---|---|
| buffer | surfactants | surfactants | Other components |
| NLS-A | 0.03-0.4 M | — | 0.3-3 M NaCl, |
| | NLS | | 0.3-3 M |
| | (0.88-11.7% | | Tris-HCl |
| | (w/v) NLS) | | |
| NLS-B | 0.03-0.4 M | 1-15% (v/v) | 0.3-3 M NaCl, |
| | NLS | Triton X-100 | 0.3-3 M |
| | (0.88-11.7% | (0.94-15.9% (w/v) | Tris-HCl |
| | (w/v) NLS) | Triton X-100) | |
| NLS-C | 0.03-0.4 M | 1-15% (v/v) | 0.3-3 M NaCl, |
| | NLS | Triton X-114 | 0.3-3 M |
| | (0.88-11.7% | (0.94-15.9% (w/v) | Tris-HCl |
| | (w/v) NLS) | Triton X-114) | |
| NLS-D | 0.03-0.4 M | 1-15% (v/v) | 0.3-3 M NaCl, |
| | NLS | Triton X-45 | 0.3-3 M |
| | (0.88-11.7% | (0.97-15.45% (w/v) | Tris-HCl |
| | (w/v) NLS) | Triton X-45) | |
| NLS-E | 0.03-0.4 M | 1-15% (v/v) | 0.3-3 M NaCl, |
| | NLS | Brij L23 | 0.3-3 M |
| | (0.88-11.7% | (0.97-15.45% | Tris-HCl |
| | (w/v) NLS) | (w/v) Brij L23) | |
| NLS-F | 0.03-0.4 M | 1-15% (v/v) | 0.3-3 M NaCl, |
| | NLS | Tween 20 | 0.3-3 M |
| | (0.88-11.7% | (0.91-16.43% | Tris-HCl |
| | (w/v) NLS) | (w/v) Tween 20) | |
| NLS-G | 0.03-0.4 M | 1-15% (v/v) | 0.3-3 M NaCl, |
| | NLS | IGEPAL CA630 | 0.3-3 M |
| | (0.88-11.7% | (0.94-15.90% (w/v) | Tris-HCl |
| | (w/v) NLS) | IGEPAL CA630) | |
| SDS-A | 0.03-0.4 M | — | — |
| | SDS | | |
| | (0.86-11.52% | | |
| | (w/v) SDS) | | |
| SDS-B | 0.03-0.4 M | 1-15% (v/v) | — |
| | SDS | Triton X-100 | |
| | (0.86-11.52% | (0.94-15.9% | |
| | (w/v) SDS) | (w/v) Triton | |
| | | X-100) | |
| SDS-C | 0.03-0.4 M | 1-15% (v/v) | — |
| | SDS | Triton X-114 | |
| | (0.86-11.52% | (0.94-15.9% (w/v) | |
| | (w/v) SDS) | Triton X-114) | |
| SDS-D | 0.03-0.4 M | 1-15% (v/v) | — |
| | SDS | Triton X-45 | |
| | (0.86-11.52% | (0.97-15.45% (w/v) | |
| | (w/v) SDS) | Triton X-45) | |
| SDS-E | 0.03-0.4 M | 1-15% (v/v) | — |
| | SDS | Brij L23 | |
| | (0.86-11.52% | (0.97-15.45% | |
| | (w/v) SDS) | (w/v) Brij L23) | |
| SDS-F | 0.03-0.4 M | 1-15% (v/v) | — |
| | SDS | Tween 20 | |
| | (0.86-11.52% | (0.91-16.43% | |
| | (w/v) SDS) | (w/v) Tween 20) | |
| SDS-G | 0.03-0.4 M | 1-15% (v/v) | — |
| | SDS | IGEPAL CA630 | |
| | (0.86-11.52% | (0.94-15.90% (w/v) | |
| | (w/v) SDS) | IGEPAL CA630) | |
| SDBS-A | 0.03-0.4 M | — | — |
| | SDBS | | |
| | (1.04-13.9% | | |
| | (w/v) SDBS) | | |
| SDBS-B | 0.03-0.4 M | 1-15% (v/v) | — |
| | SDBS | Triton X-100 | |
| | (1.04-13.9% | (0.94-15.9% | |
| | (w/v) SDBS) | (w/v) Triton | |
| | | X-100) | |
| SDBS-C | 0.03-0.4 M | 1-15% (v/v) | — |
| | SDBS | Triton X-114 | |
| | (1.04-13.9% | (0.94-15.9% (w/v) | |
| | (w/v) SDBS) | Triton X-114) | |
| SDBS-D | 0.03-0.4 M | 1-15% (v/v) | — |
| | SDBS | Triton X-45 | |
| | (1.04-13.9% | (0.97-15.45% (w/v) | |
| | (w/v) SDBS) | Triton X-45) | |
| SDBS-E | 0.03-0.4 M | 1-15% (v/v) | — |
| | SDBS | Brij L23 | |
| | (1.04-13.9% | (0.97-15.45% | |
| | (w/v) SDBS) | (w/v) Brij L23) | |
| SDBS-F | 0.03-0.4 M | 1-15% (v/v) | — |
| | SDBS | Tween 20 | |
| | (1.04-13.9% | (0.91-16.43% | |
| | (w/v) SDBS) | (w/v) Tween 20) | |
| SDBS-G | 0.03-0.4 M | 1-15% (v/v) | — |
| | SDBS | IGEPAL CA630 | |
| | (1.04-13.9% | (0.94-15.90% (w/v) | |
| | (w/v) SDBS) | IGEPAL CA630) | |
| SC-A | 0.03-0.4 M SC | — | — |
| | (1.29-17.24% | | |
| | (w/v) SC) | | |
| SC-B | 0.03-0.4 M SC | 1-15% (v/v) | — |
| | (1.29-17.24% | Triton X-100 | |
| | (w/v) SC) | (0.94-15.9% | |
| | | (w/v) Triton | |
| | | X-100) | |
| SC-C | 0.03-0.4 M SC | 1-15% (v/v) | — |
| | (1.29-17.24% | Triton X-114 | |
| | (w/v) SC) | (0.94-15.9% (w/v) | |
| | | Triton X-114) | |
| SC-D | 0.03-0.4 M SC | 1-15% (v/v) | — |
| | (1.29-17.24% | Triton X-45 | |
| | (w/v) SC) | (0.97-15.45% | |
| | | (w/v) Triton | |
| | | X-45) | |
| SC-E | 0.03-0.4 M SC | 1-15% (v/v) | — |
| | (1.29-17.24% | Brij L23 | |
| | (w/v) SC) | (0.97-15.45% | |
| | | (w/v) Brij | |
| | | L23) | |
| SC-F | 0.03-0.4 M SC | 1-15% (v/v) | — |
| | (1.29-17.24% | Tween 20 | |
| | (w/v) SC) | (0.91-16.43% | |
| | | (w/v) Tween 20) | |
| SC-G | 0.03-0.4 M SC | 1-15% (v/v) | — |
| | (1.29-17.24% | IGEPAL CA630 | |
| | (w/v) SC) | (0.94-15.90% | |
| | | (w/v) IGEPAL | |
| | | CA630) | |

### Example 2: Concentration and Purification of DNA from Sample Lysates

### Sequential Aqueous Two-Phase Systems (ATPS)

In this example, the extraction procedure involves two sequential aqueous two-phase systems (ATPS), including a first ATPS and a second ATPS to isolate, purify and concentrate DNA from sample lysates of plasma samples prepared in Example 1. Many types of first ATPS compositions and second ATPS compositions can be used. In some embodiments, the first ATPS composition and/or second ATPS composition is purchased from Phase Scientific. In some embodiments, the first ATPS composition and/or the second ATPS composition is selected from Table 3.

TABLE 3
Example ATPS compositions. All concentrations
are shown in w/v ratio unless otherwise noted.

| ATPS | Composition |
|---|---|
| 1 | 3-18% PEG 6000, 1-6% dextran 450-650 kDa, |
| | 5-50 mM Na₂HPO₄/NaH₂PO₄ |
| 2 | 1-9% PEG 6000, 17-25% dextran 2,200,000 Da |
| 3 | 9-18% PEG 6000, 9-17% dextran 2,200,000 Da |
| 4 | 18-25% PEG 6000, 0.5-9% dextran 2,200,000 Da |
| 5 | 1-9% PEG 6000, 20-30% dextran 460,000 Da |
| 6 | 9-18% PEG 6000, 9-20% dextran 460,000 Da |
| 7 | 18-25% PEG 6000, 0.5-9% dextran 460,000 Da |
| 8 | 1-9% PEG 6000, 17-25% dextran 179,000 Da |
| 9 | 9-18% PEG 6000, 9-17% dextran 179,000 Da |
| 10 | 18-25% PEG 6000, 0.5-9% dextran 179,000 Da |
| 11 | 1-9% PEG 8000, 17-25% dextran 70,000 Da |
| 12 | 9-18% PEG 8000, 9-17% dextran 70,000 Da |
| 13 | 18-25% PEG 8000, 0.5-9% dextran 70,000 Da |
| 14 | 1-16% PEG 6000, 33-50% dextran 3,400 Da |
| 15 | 16-33% PEG 6000, 16-33% dextran 3,400 Da |
| 16 | 33-50% PEG 6000, 1-16% dextran 3,400 Da |
| 17 | 1-16% PEG 4000, 33-50% dextran 3,400 Da |
| 18 | 16-33% PEG 4000, 16-33% dextran 3,400 Da |
| 19 | 33-50% PEG 4000, 1-16% dextran 3,400 Da |
| 20 | 1-5% PEG 20000, 20-30% dextran 500,000 Da |
| 21 | 6-11% PEG 20000, 9-20% dextran 500,000 Da |
| 22 | 11-15% PEG 20000, 0.5-9% dextran 500,000 Da |
| 23 | 1-7% PEG 8000, 20-30% dextran 500,000 Da |
| 24 | 7-14% PEG 8000, 9-20% dextran 500,000 Da |
| 25 | 14-20% PEG 8000, 0.5-9% dextran 500,000 Da |
| 26 | 2-16% PEG 3400, 20-30% dextran 500,000 Da |
| 27 | 16-33% PEG 3400, 9-20% dextran 500,000 Da |
| 28 | 33-50% PEG 3400, 0.5-9% dextran 500,000 Da |
| 29 | 0.1-7% methylcellulose^{[1]}, 14-20% dextran 2,200,000 Da |
| 30 | 7-14% methylcellulose^{[1]}, 7-14% dextran 2,200,000 Da |
| 31 | 14-20% methylcellulose^{[1]}, 0.1-7% dextran 2,200,000 Da |
| 32 | 0.5-16% PolyViol 28/20^{[2]}, 33-50% dextran 2,200,000 Da |
| 33 | 16-33% PolyViol 28/20^{[2]}, 16-33% dextran 2,200,000 Da |
| 34 | 33-50% PolyViol 28/20^{[2]}, 0.5-16% dextran 2,200,000 Da |
| 35 | 4-21% PEG 600, 58-70% PPG 400 |
| 36 | 21-42% PEG 600, 36-58% PPG 400 |
| 37 | 42-60% PEG 600, 4-36% PPG 400 |
| 38 | 0.5-7% hydroxypropyl dextran 70, 14-20% dextran |
| | 2,200,000 Da |
| 39 | 7-14% hydroxypropyl dextran 70, 7-14% dextran |
| | 2,200,000 Da |
| 40 | 14-20% hydroxypropyl dextran 70, 0.5-7% dextran |
| | 2,200,000 Da |
| 41 | 0.5-7% PEG 8000, 20-30% hydroxypropyl starch 60-70 kDa |
| 42 | 7-14% PEG 8000, 10-20% hydroxypropyl starch 60-70 kDa |
| 43 | 14-20% PEG 8000, 1-10% hydroxypropyl starch 60-70 kDa |
| 44 | 10-20% Triton X-100 |
| 45 | 20-30% Triton X-100 |
| 46 | 30-40% Triton X-100 |
| 47 | 5-18% Triton X-114 |
| 48 | 18-29% Triton X-114 |
| 49 | 29-40% Triton X-114 |
| 50 | 60-90% Triton X-114, 5-17% Triton X-45 |
| 51 | 3-19% C10E4 (decy tetraethylene glycol ether) |
| 52 | 19-34% C10E4 (decy tetraethylene glycol ether) |
| 53 | 34-50% C10E4 (decy tetraethylene glycol ether) |
| 54 | 1-10% Pluronic F68, 14-20% potassium phosphate |
| 55 | 10-20% Pluronic F68, 7-14% potassium phosphate |
| 56 | 20-30% Pluronic F68, 1-7% potassium phosphate |
| 57 | 5-21% Pluronic 17R4, 7-10% potassium phosphate |
| 58 | 21-42% Pluronic 17R4, 3-7% potassium phosphate |
| 59 | 42-60% Pluronic 17R4, 1-3% potassium phosphate |
| 60 | 2-21% Pluronic L-35, 14-20% sodium sulfate |
| 61 | 21-42% Pluronic L-35, 7-14% sodium sulfate |
| 62 | 42-60% Pluronic L-35, 1-7% sodium sulfate |
| 63 | 8-18% PEG 600, 7-20% K₂HPO₄, 0.1-5% KH₂PO₄ |
| 64 | 8-30% PEG 600, 1-6% K₂HPO₄, 12-20% KH₂PO₄ |
| 65 | 8-25% PEG 200, 40-50% K₂HPO₄, 4-16% KH₂PO₄ |
| 66 | 8-18% PEG 600, 7-20% K₂HPO₄, 0.1-5% KH₂PO₄ |
| 67 | 6-18% PEG 600, 2-12% K₂HPO₄, 0.1-5% KH₂PO₄, |
| | 0.01-10% (v/v) Triton X-114 |
| 68 | 6-18% PEG 600, 2-12% K₂HPO₄, 0.1-5% KH₂PO₄ |
| 69 | 5-20% PEG 600, 0.1-6% K₂HPO₄, 6-18% KH₂PO₄ |
| 70 | 5-20% PEG 600, 2-12% K₂HPO₄, 0.1-5% KH₂PO₄ |
| 71 | 5-17% PEG 200, 3-12% K₂HPO₄, 0.1-5% KH₂PO₄ |
| 72 | 5-17% PEG 200, 0.1-4% K₂HPO₄, 5-16% KH₂PO₄ |
| 73 | 5-15% PEG 200, 3-13% K₂HPO₄, 0.1-5% KH₂PO₄ |
| 74 | 5-15% PEG 200, 0.1-4% K₂HPO₄, 6-20% KH₂PO₄ |
| 75 | 36-60% PPG 425, 0.1-5% K₂HPO₄, 0.1-3% KH₂PO₄ |
| 76 | 40-60% PPG 425, 0.1-4% K₂HPO₄, 0.1-5% KH₂PO₄ |
| 77 | 40-50% PPG 425, 0.1-4% K₂HPO₄, 0.1-5% KH₂PO₄ |
| 78 | 36-60% PPG 425, 0.1-3% K₂HPO₄, 0.1-5% KH₂PO₄ |
| 79 | 3-20% PEG 200, 10-30% K₂HPO₄, 1-9% KH₂PO₄ |
| 80 | 3-13% PEG 200, 3-12% K₂HPO₄, 0.1-5% KH₂PO₄ |
| 81 | 3-13% PEG 200, 1-4% K₂HPO₄, 5-20% KH₂PO₄ |
| 82 | 3-13% PEG 200, 13-20% K₂HPO₄, 1-7% KH₂PO₄ |
| 83 | 3-13% PEG 200, 0.1-12% K₂HPO₄, 16-36% KH₂PO₄ |
| 84 | 3-12% PEG 200, 2-8% K₂HPO₄, 20-40% KH₂PO₄ |
| 85 | 3-12% PEG 200, 10-30% K₂HPO₄, 1-7% KH₂PO₄ |
| 86 | 30-50% PEG-ran-PPG^{[4]}, 0.1-5% K₂HPO₄, 0.1-3% KH₂PO₄ |
| 87 | 2-9% PEG 200, 8-20% K₂HPO₄, 1-7% KH₂PO₄ |
| 88 | 2-9% PEG 200, 5-20% K₂HPO₄, 0.1-4% KH₂PO₄ |
| 89 | 2-9% PEG 200, 2-8% K₂HPO₄, 20-40% KH₂PO₄ |
| 90 | 2-9% PEG 200, 2-8% K₂HPO₄, 16-36% KH₂PO₄ |
| 91 | 2-9% PEG 200, 1-8% K₂HPO₄, 20-40% KH₂PO₄ |
| 92 | 2-9% PEG 200, 1-7% K₂HPO₄, 8-32% KH₂PO₄ |
| 93 | 2-9% PEG 200, 10-30% K₂HPO₄, 1-7% KH₂PO₄ |
| 94 | 2-8% PEG 200, 4-13% K₂HPO₄, 0.1-5% KH₂PO₄ |
| 95 | 2-8% PEG 200, 1-5% K₂HPO₄, 6-20% KH₂PO₄ |
| 96 | 2-12% PEG 200, 5-20% Na₂SO₄, 0.01-0.1M Na₂HPO₄, |
| | 0.01-0.1M NaH₂PO₄ |
| 97 | 20-40% PEG 600, 2-12% K₂HPO₄, 0.1-5% KH₂PO₄ |
| 98 | 20-40% PEG 600, 0.1-4% K₂HPO₄, 2-9% KH₂PO₄ |
| 99 | 20-40% PEG 1000, 1-12% K₂HPO₄, 0.1-3% KH₂PO₄ |
| 100 | 20-40% PEG 1000, 0.1-4% K₂HPO₄, 3-12% KH₂PO₄ |
| 101 | 20-40% PEG 1000, 0.1-4% K₂HPO₄, 2-9% KH₂PO₄ |
| 102 | 16-36% PEG 600, 2-12% Na₂SO₄, 0.01-0.1M Na₂HPO₄, |
| | 0.01-0.1M NaH₂PO₄ |
| 103 | 1-6% PEG 200, 3-12% K₂HPO₄, 25-50% KH₂PO₄ |
| 104 | 1-6% PEG 200, 0.1-5% K₂HPO₄, 6-36% KH₂PO₄ |
| 105 | 1-6% PEG 200, 0.1-5% K₂HPO₄, 5-20% KH₂PO₄ |
| 106 | 11-33% PEG 3000, 0.1-6% K₂HPO₄, 0.1-3% KH₂PO₄ |
| 107 | 11-33% PEG 3000, 0.1-4% K₂HPO₄, 1-8% KH₂PO₄ |
| 108 | 10-32% PEG-ran-PPG^{[3]}, 0.1-4% citric acid, 3-12% |
| | sodium citrate |
| 109 | 10-32% PEG-ran-PPG^{[3]}, 0.1-4% citric acid, 2-9% |
| | sodium citrate |
| 110 | 10-32% PEG-ran-PPG^{[3]}, 0.1-3% citric acid, 1-6% |
| | sodium citrate |
| 111 | 10-32% PEG-ran-PPG^{[3]}, 0.1-3% citric acid, 0.1-4% |
| | sodium citrate |
| 112 | 10-32% PEG 8000, 0.1-6% K₂HPO₄, 0.1-3% KH₂PO₄ |
| 113 | 10-32% PEG 8000, 0.1-4% K₂HPO₄, 2-8% KH₂PO₄ |
| 114 | 10-32% PEG 8000, 0.1-4% K₂HPO₄, 0.1-9% KH₂PO₄ |
| 115 | 10-30% PEG-ran-PPG^{[4]}, 0.1-4% K₂HPO₄, 0.1-6% KH₂PO₄ |
| 116 | 10-30% PEG-ran-PPG^{[4]}, 0.1-3% K₂HPO₄, 2-8% KH₂PO₄ |
| 117 | 10-25% polyvinyl pyrrolidone 3,500 Da, 15-20% |
| | potassium phosphate |
| 118 | 25-35% polyvinyl pyrrolidone 3,500 Da, 10-15% |
| | potassium phosphate |
| 119 | 35-50% polyvinyl pyrrolidone 3,500 Da, 5-10% |
| | potassium phosphate |
| 120 | 2-12% PEG 600, 35-50% sodium phosphate |
| 121 | 12-18% PEG 600, 20-35% sodium phosphate |
| 122 | 18-25% PEG 600, 10-20% sodium phosphate |
| 123 | 5-20% polyvinyl pyrrolidone 10,000 Da, 30-40% |
| | sodium citrate |
| 124 | 20-35% polyvinyl pyrrolidone 10,000 Da, 20-30% |
| | sodium citrate |
| 125 | 35-50% polyvinyl pyrrolidone 10,000 Da, 10-20% |
| | sodium citrate |
| 126 | 2-10% PEG 300, 49-60% sodium citrate |
| 127 | 10-18% PEG 300, 37-49% sodium citrate |
| 128 | 18-25% PEG 300, 20-37% sodium citrate |
| 129 | 10-35% PPG 425, 14-20% sodium sulfate |
| 130 | 35-50% PPG 425, 8-14% sodium sulfate |
| 131 | 55-70% PPG 425, 3-8% sodium sulfate |
| 132 | 5-16% PPG 425, 11-15% sodium polyacrylate 240,000 |
| 133 | 16-24% PPG 425, 5-11% sodium polyacrylate 240,000 |
| 134 | 24-40% PPG 425, 0.5-5% sodium polyacrylate 240,000 |
| 135 | 4-12% PEG 8000, 21-30% sodium carbonate |
| 136 | 12-21% PEG 8000, 11-21% sodium carbonate |
| 137 | 21-30% PEG 8000, 3-11% sodium carbonate |
| 138 | 1-5% PEG 8000, 14-20% sodium polyacrylate 16,000 |
| 139 | 6-11% PEG 8000, 7-14% sodium polyacrylate 16,000 |
| 140 | 11-15% PEG 8000, 2-7% sodium polyacrylate 16,000 |
| 141 | 10-25% polyvinyl alcohol 3,500 Da, 7-10% potassium |
| | phosphate |
| 142 | 25-35% polyvinyl alcohol 3,500 Da, 4-7% potassium |
| | phosphate |
| 143 | 35-50% polyvinyl alcohol 3,500 Da, 2-4% potassium |
| | phosphate |
| 144 | 2-5% PEG 400, 34-50% potassium phosphate |
| 145 | 6-11% PEG 400, 22-34% potassium phosphate |
| 146 | 11-15% PEG 400, 10-22% potassium phosphate |
| 147 | 4-12% polyvinyl alcohol 8,000 Da, 21-30% sodium |
| | citrate |
| 148 | 12-21% polyvinyl alcohol 8,000 Da, 11-21% sodium |
| | citrate |
| 149 | 21-30% polyvinyl alcohol 8,000 Da, 3-11% sodium |
| | citrate |
| 150 | 1-5% PPG 425, 20-30% sodium citrate |
| 151 | 6-11% PPG 425, 9-20% sodium citrate |
| 152 | 11-15% PPG 425, 0.5-9% sodium citrate |
| 153 | 2-12% polyvinyl alcohol 20,000 Da, 21-30% |
| | sodium sulfate |
| 154 | 12-21% polyvinyl alcohol 20,000 Da, 11-21% |
| | sodium sulfate |
| 155 | 21-30% polyvinyl alcohol 20,000 Da, 3-11% |
| | sodium sulfate |
| 156 | 0.5-18% PEG-ran-PPG 12,000 Da, 14-20% |
| | sodium sulfate |
| 157 | 18-35% PEG-ran-PPG 12,000 Da, 7-14% |
| | sodium sulfate |
| 158 | 35-50% PEG-ran-PPG 12,000 Da, 2-7% |
| | sodium sulfate |

^{[1]}provided as 4000 cP @ 2% solution
^{[2]}polyvinyl alcohol characterized as 97-99% hydrolysis, viscosity cP (@ 4% solution @ 20° C.) = 20-30, and degree of polymerization = 1700-2000
^{[3]}PEG-ran-PPG having an average molecular weight of about 2,500 Da and an EO:PO ratio of about 75:25
^{[4]}PEG-ran-PPG having an average molecular weight of about 12,000 Da and an EO:PO ratio of about 75:25

2.22 mL of each lysate prepared in Example 1 was added to the first ATPS. A dye was added to visualize the two phases, which resulted in a colored top phase. In the first ATPS, DNA partitions to the bottom phase (target-rich phase) and proteins partition to the top phase (target-poor phase). The solution was forced to become turbid by vigorous vortex for 15 seconds. Phase separation was then facilitated by centrifuge at 2300 rcf for 6 minutes. After phase separation, the bottom phase, which amount to around 1 mL, was extracted carefully and transferred to the second ATPS. The mixture was vortexed for 15 seconds to yield a turbid solution and was centrifuged at 7000 rcf for 1 minute to achieve phase separation. The top phase and the bottom phase volumes in the resulting solution was around 150 μL and 1150 μL, respectively. DNA partitions to the top phase (target-rich phase) of the second ATPS, which was extracted carefully for further purification and detection.

### Purification of DNA

In this example, the purification of DNA was done by magnetic beads extraction. The top phase from the second ATPS was transferred to a tube containing 800 μL of binding buffer. 12 μL of MagQu magnetic beads was added to the tube, and the sample was incubated with tilt rotation for 5 minutes. The tube was then briefly spun down and placed on a magnetic stand for 2 minutes to immobilize the beads at the tube wall. The supernatant was pipetted and discarded without disturbing the magnetic beads. 800 μL of binding buffer was added to the sample. The tubes were rotated on the rack for 720° in total. The supernatant was pipetted and discarded. 800 μL of washing buffer (70% ethanol, 0.001 M EDTA, 0.01 M Tris-HCl) was added to the sample, and the tube was rotated on the rack for 720° in total. The supernatant was pipetted and discarded. The washing was performed twice, and the tube was placed on the rack with cap opened to allow the bead to dry. 40 μL of elution buffer (0.01 M Tris-HCl, 0.001 M EDTA) was added to the sample after drying. The beads complex was resuspended by continuous pipette mixing, followed by mild vortex. The sample was incubated at room temperature for 3 minutes and was briefly spun down. The tube was then placed on the magnetic rack for 1 minute. The supernatant was collected carefully without disturbing the magnetic beads for detection.

### Detection of DNA

Recovery of DNA spike in the extracted samples were quantified by quantitative real-time polymerase chain reaction (qPCR) using the Quant Studio 5. qPCR master mix was prepared as follows per reaction: 5 μL of Taqman Fast Advanced Master Mix (Applied Biosystems, Ref: 4444557), 0.5 μL of 20× custom pre-mixed custom oligo PSI-145 FAM Dental, 0.4 μL of Universal Spike II Primer (TATAA, DS25SII), 0.2 μL Universal Spike II Probe (TATAA, DSSII), 1.9 μL of Ultra-Pure water. The total DNA extraction yields were evaluated using Qubit 4.0 fluorometer (Thermo Scientific), with Qubit 1× dsDNA HS Assay Kit.

### Results

### Observations

To evaluate the effect of surfactants in preventing the debris layer formation in the ATPS system, various combinations of anionic and non-ionic surfactants in lysis buffer as listed in Table 2 have been tested.

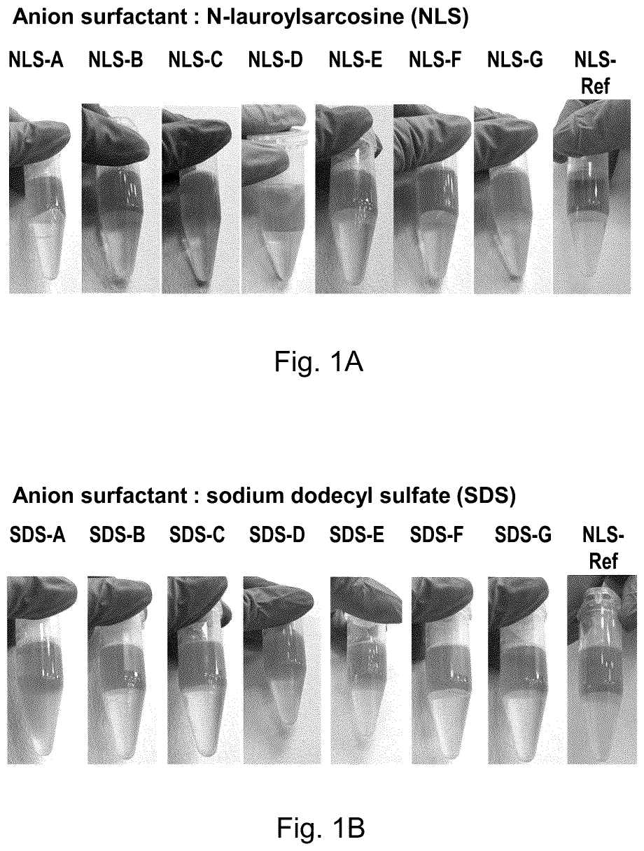

Now referring to FIGS. 1A-1D, which show the ATPS-sample lysate mixtures after phase separation in the first ATPS, in which the sample lysates were prepared from example lysis buffer compositions according to Table 2. The observations are summarized in Table 4. The mixing of first ATPS with sample lysates prepared from lysis buffers NLS-A, SDS-A, SDBS-A and SC-A (i.e. lysis buffers that do not contain non-ionic surfactants) resulted in a thick debris layer in the interface, where all were rated a debris layer score of 5. Reference samples (NLS-Ref, SDS-Ref, SDBS-Ref and SC-Ref) containing a non-ionic surfactant with the lowest HLB (such as Span 80, with HLB value of 4.3) also resulted in very large debris layers, where all were rated a debris layer score of 5. The combination of NLS with Triton X-45 (NLS-D), SDBS with Triton X-45 (SDBS-D) and sodium cholate with Triton X-45 (SC-D) resulted in a debris layer in the top phase, while the combination of sodium cholate with Triton X-114 (SC-C) and sodium cholate with IGEPAL CA630 (SC-G) gave rise to a thinner layer of proteins. Other combinations (NLS-B, C, E, F, G; SDS-B-G; SDBS-B, C, E-G; SC-B, E, F) resulted in very thin debris layer, in which the combination of NLS with Brij L23 (NLS-E) and IGEPAL CA630 (NLS-G), SDS with Triton X-100 (SDS-B) and Triton X-114 (SDS-C), and sodium cholate with Brij L23 (SC-E) gave rise to almost no debris layer. The observations are summarized in Table 3. When compared to lysis buffers that do not contain non-ionic surfactants (NLS-A, SDS-A, SDBS-A and SC-A) and/or the reference samples (NLS-Ref, SDS-Ref, SDBS-Ref and SC-Ref), most example combinations (NLS-B-G; SDS-B-G; SDBS-B-G; SC-B, E-G) show significant results in reducing the debris layer and were rated at debris layer scores ranging from 1-3.

These results highlight the importance of having both anionic and non-ionic surfactants in the digestion of proteins during lysis. When using lysis buffer compositions that do not contain non-ionic surfactants to treat the samples, the resulting sample lysates will form a thick debris layer when mixing with the ATPS. The chunk of proteins affects the extraction of bottom phase, and therefore is not desirable. The lysis buffer compositions with the combination of anionic surfactants with non-ionic surfactants with low HLB values (such as Triton X-45 with HLB value of 9.8, and Span80 in the reference samples with HLB value of 4.3) demonstrate the lowest capacity to prevent the debris layer formation within the ATPS. On the other hand, the lysis buffer compositions with the combination of anionic surfactants with non-ionic surfactants with higher HLB values (Triton X-100, Triton X-114, Brij L23, Tween 20 and IGEPAL CA630) can effectively digest the proteins and prevent the debris layer formation within the ATPS.

TABLE 4
Observation of debris layers formation using different lysis buffers

| | Debris | | | Debris | |
|---|---|---|---|---|---|
| Lysis | layer | | Lysis | layer | |
| buffer | score | Observations | buffer | score | Observations |
| NLS-A | 5 | thick in | SDBS-A | 5 | thick in middle |
| | | middle | | | |
| NLS-B | 2 | little in | SDBS-B | 2 | little in middle |
| | | middle | | | |
| NLS-C | 2 | little in | SDBS-C | 2 | little in middle |
| | | middle | | | |
| NLS-D | 3 | thick on | SDBS-D | 3 | little in middle, |
| | | top | | | thick on top |
| NLS-E | 1 | almost none | SDBS-E | 2 | little in middle |
| NLS-F | 2 | little in | SDBS-F | 2 | little in middle |
| | | middle | | | |
| NLS-G | 1 | almost none | SDBS-G | 2 | little in middle |
| NLS-Ref | 5 | thick in | SDBS-Ref | 5 | thick on top |
| | | middle | | | |
| SDS-A | 5 | very thick | SC-A | 5 | thick in middle |
| | | in middle | | | |
| SDS-B | 1 | almost none | SC-B | 2 | little in middle |
| SDS-C | 1 | almost none | SC-C | 4 | some in middle |
| SDS-D | 2 | little in | SC-D | 5 | thick in middle |
| | | middle and | | | and top |
| | | top | | | |
| SDS-E | 2 | little in | SC-E | 1 | almost none |
| | | middle | | | |
| SDS-F | 2 | little in | SC-F | 2 | little in middle |
| | | middle | | | |
| SDS-G | 2 | little in | SC-G | 3 | some in middle |
| | | middle | | | |
| SDS-Ref | 5 | thick in | SC-Ref | 5 | thick in middle |
| | | middle | | | |

5: Very thick visible debris layers in interface
4: Thick visible debris layers in interface
3: Visible debris layer in interface or top phase
2: Very thin debris layer in interface
1: Almost no debris layer observed

### Example 3: Evaluation of DNA Extraction Efficiency

### Extraction Efficiency

To further evaluate the efficiency of extraction of DNA in sequential ATPS systems using lysis buffers which form lesser or almost no debris layer as demonstrated in Example 2, lysis buffers NLS-E, NLS-G, SDS-C, SDS-D, SDBS-C, SDBS-G, SC-B and SC-F were employed in the extraction of DNA of EDTA plasma.

The samples containing 100 fg of 145 bp dsDNA in 2 mL human plasma were prepared, amounting to 2.5 fg per μL of eluant assuming 100% recovery. The positive control was prepared by spiking appropriate amount of 145 bp dsDNA into the respective elution volume to form 2.5 fg DNA per μL of eluant, which represents the 100% recovery condition. The steps for lysis of samples and extraction of DNA using sequential ATPS were the same or similar to the steps as described in the preceding examples.

The extraction efficiency was quantified by qPCR. The extraction efficiency of cfDNA present in the plasma was quantified by Qubit.

### Results

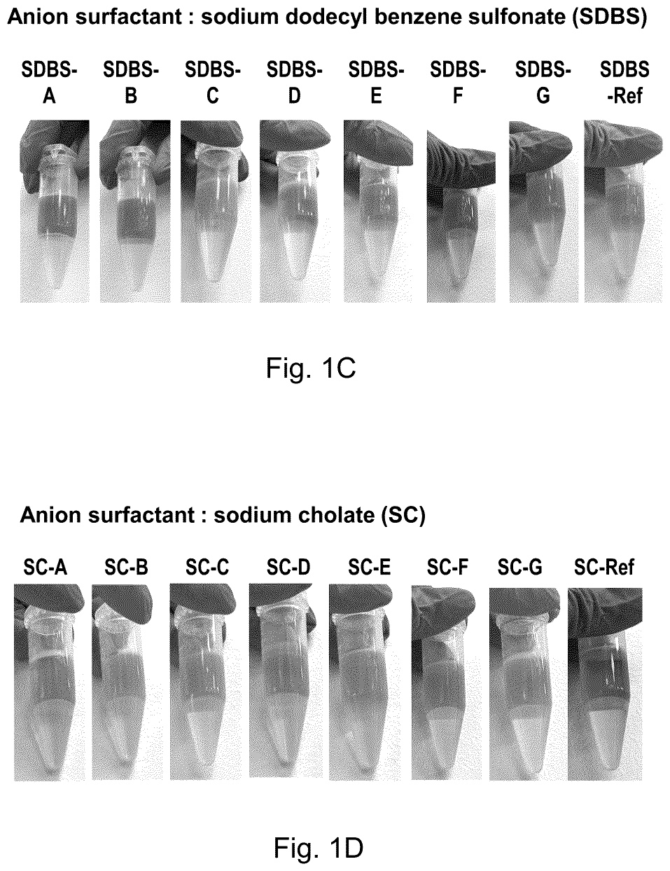

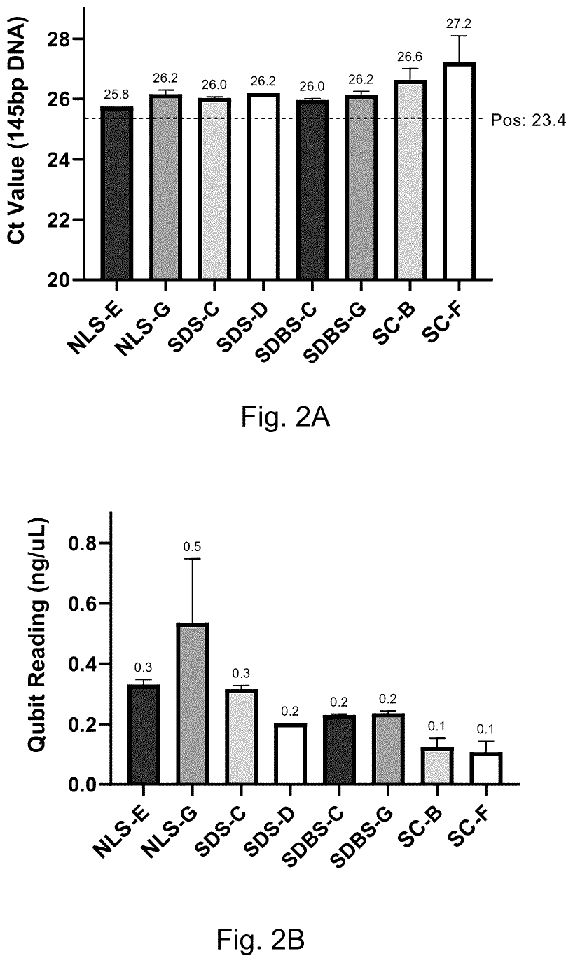

The results of extraction efficiency as quantified by qPCR are summarized in FIG. 2A and Table 5. The cycle threshold (Ct) values generated were found to be similar among the samples, with a difference from the positive control of 0.4 to 1.9 Ct values, indicative of satisfactory recovery of 145 bp dsDNA from the lysate. The results suggested that DNA can be extracted effectively by ATPS without substantial debris layer formation using lysates prepared from lysis buffers with suitable combinations of anionic surfactants and non-ionic surfactants with higher HLB values.

TABLE 5
Extraction efficiency quantified by qPCR (145 bp DNA)

| Lysis Buffer | NLS-E | NLS-G | SDS-C | SDS-D | SDBS-C | SDBS-G | SC-B | SC-F |
|---|---|---|---|---|---|---|---|---|
| CT value | 25.75 | 26.26 | 26.06 | * | 25.94 | 26.08 | 26.90 | 26.59 |
| (145 bp) | 25.75 | 26.07 | 26.01 | 26.19 | 26.00 | 26.22 | 26.37 | 27.84 |
| Average ± | 25.75 ± 0.00 | 26.17 ± 0.14 | 26.04 ± 0.03 | 26.19 | 25.97 ± 0.04 | 26.15 ± 0.1 | 26.63 ± 0.38 | 27.22 ± 0.89 |
| SD | | | | | | | | |
| Difference | 0.3967 | 0.8167 | 0.6867 | 0.837 | 0.6167 | 0.7967 | 1.2767 | 1.8667 |
| from | | | | | | | | |
| positive | | | | | | | | |
| control** | | | | | | | | |

* Datapoint removed from SDS-D (SDS + Triton X-45) due to user operator error
**CT value for Positive Control = 25.353 ± 0.06.

The results of extraction efficiency of cfDNA present in the plasma as quantified by Qubit are summarized in FIG. 2B and Table 6. As the amount of spike in dsDNA was small and out of the detection range of Qubit, the Qubit readings corresponds only to the DNA released from the plasma sample during lysis, which is mostly cfDNA and genomic DNA. As shown in FIG. 2B and Table 6, the Qubit readings for sample using different lysis buffer were found to be in the range of 0.1 to 0.5 ng/μL. The variation between different samples may indicate the effectiveness of extraction of cfDNA in plasma with the presence of various surfactants. The results showed that cfDNA can be extracted effectively by ATPS without substantial debris layer formation using lysates prepared from lysis buffers with suitable combinations of anionic surfactants and non-ionic surfactants with higher HLB values.

TABLE 6
Qubit detection of dsDNA recovery of lysates prepared from selected lysis buffers

| Lysis Buffer | NLS-E | NLS-G | SDS-C | SDS-D | SDBS-C | SDBS-G | SC-B | SC-F |
|---|---|---|---|---|---|---|---|---|
| Qubit | 0.319 | 0.387 | 0.324 | * | 0.227 | 0.230 | 0.103 | 0.132 |
| Reading | 0.343 | 0.686 | 0.307 | 0.203 | 0.232 | 0.241 | 0.144 | 0.080 |
| (ng/uL) | | | | | | | | |
| Average ± | 0.331 | 0.537 | 0.316 | 0.203 | 0.230 | 0.236 | 0.124 | 0.106 |
| SD | | | | | | | | |

* out of range result is removed from sample SDS-D (SDS + Triton X-45) due to user operator error

The exemplary embodiments of the present invention are thus fully described. Although the description referred to particular embodiments, it will be clear to one skilled in the art that the present invention may be practiced with variation of these specific details. Hence this invention should not be construed as limited to the embodiments set forth herein.

### NUMBERED EMBODIMENTS

Embodiment 1. A method for preventing debris layer formation during phase separation in an ATPS-sample lysate mixture, comprising the steps of: (a) mixing and incubating a biological sample comprising nucleic acid components and proteins with a lysing composition to form a sample lysate, wherein the composition comprises: (i) at least one non-ionic surfactant with a hydrophilic-lipophilic balance (HLB) value greater than or equal to 10; and (ii) at least one anionic surfactant, wherein the anionic surfactant comprises a cation and an anionic group; wherein the anionic group is selected from the group consisting of a cholate, a carboxylate, a sulfonate, a sulfate, and a phosphate ester; wherein proteins are substantially digested in the sample lysates; (b) adding the sample lysate to an aqueous two-phase system (ATPS) composition comprising a polymer, a salt, a surfactant, or any combination thereof dissolved in an aqueous solution to form the ATPS-sample lysate mixture, wherein the ATPS-sample lysate mixture separates into a target-rich phase solution and a target-poor phase solution such that the nucleic acid components partition selectively to the target-rich phase solution without substantial debris layer formation.

Embodiment 2. The method of embodiment 1, further comprising the steps of (c) optionally collecting the first target-rich phase solution and mixing the first target-rich phase solution with a second ATPS composition comprising a polymer, a salt, a surfactant, or any combination thereof dissolved in an aqueous solution to form a second target-rich phase solution and a second target-poor phase solution, such that the nucleic acid components partition to the second target-rich phase solution without substantial debris layer formation; and (d) isolating the nucleic acid components from the target-rich phase solution and/or the second target-rich phase solution.

Embodiment 3. The method of embodiment 2, wherein step (d) further comprises the following steps: mixing the target-rich phase solution or the second target-rich phase solution with a binding buffer to form a mixed solution; loading the mixed solution onto a purification system configured to selectively extract and purify the nucleic acid components; and eluting and collecting the nucleic acid components from the purification system, resulting in a final solution containing the concentrated and purified nucleic acid components.

Embodiment 4. The method of embodiment 3, wherein the purification system is a solid phase extraction column, liquid chromatography column, or magnetic beads.

Embodiment 5. The method of any one of the preceding embodiments, wherein the hydrophilic-lipophilic balance (HLB) value of the at least one non-ionic surfactant is between 10 and 20.

Embodiment 6. The method of any one of the preceding embodiments, wherein the hydrophilic-lipophilic balance (HLB) value of the at least one non-ionic surfactant is between 10 and 17.

Embodiment 7. The method of any one of the preceding embodiments, wherein the hydrophilic-lipophilic balance (HLB) value of the at least one non-ionic surfactant is between 12.4 and 16.9.

Embodiment 8. The method of any one of the preceding embodiments, wherein the hydrophilic-lipophilic balance (HLB) value of the at least one non-ionic surfactant is 12.4, 13.4, 16.7 or 16.9.

Embodiment 9. The method of any one of the preceding embodiments, wherein the at least one non-ionic surfactant comprises at least one —(OCH₂CH₂)ₙ— chain and at least one OH group, wherein n is a whole number between 2 and 40.

Embodiment 10. The method of embodiment 9, wherein the at least one non-ionic surfactant is selected from the group consisting of ethoxylated natural fatty alcohol, octylphenoxy alcohol, ethoxylated sorbitan ester, and any combination thereof.

Embodiment 11. The method of embodiment 10, wherein the ethoxylated natural fatty alcohol is a compound of Formula III: CH₃(CH₂)₁₀CH₂(OCH₂CH₂)ₙOH (Formula III), wherein n is 10-23.

Embodiment 12. The method of embodiment 10, wherein the octylphenoxy alcohol comprises one or more compounds of Formula I:

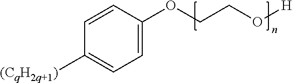

(Formula I), wherein n is 6, 7, 8, 9, or 10, and q is 6, 7, 8, 9, 10, 11, or 12.

Embodiment 13. The method of embodiment 12, wherein n is 7 or 8 or a mixture thereof; or 9 or 10 or a mixture thereof.

Embodiment 14. The method of embodiment 12, wherein n is 9, 10, or a mixture thereof; and the average molecular weight of the octylphenoxy alcohol is about 603 g/mol or about 625 g/mol.

Embodiment 15. The method of embodiment 10, wherein the ethoxylated sorbitan ester is a compound of Formula II:

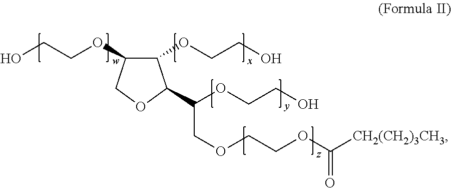

wherein w+x+y+z=20

Embodiment 16. The method of embodiment 1, wherein the at least one non-ionic surfactant is selected from the group consisting of Triton X-100, Triton X-114, Brij L23, Tween 20 and IGEPAL CA630.

Embodiment 17. The method of any one of the preceding embodiments, wherein the sulfonate is selected from the group consisting of petroleum sulfonate, alkylbenzenesulfonate, naphthalenesulfonate, olefin sulfonate, and any combination thereof; and wherein the sulfate is selected from the group consisting of alkyl sulfate, sulfated natural oil, sulfated natural fat, sulfated ester, sulfated alkanolamide, sulfated alkylphenol, and any combination thereof.

Embodiment 18. The method of any one of the preceding embodiments, wherein the cholate is sodium cholate, the carboxylate is sodium N-lauroyl sarcosine (NLS), the sulfate is sodium dodecyl sulfate (SDS), and the sulfonate is sodium dodecyl benzene sulfonate (SDBS).

Embodiment 19. The method of any one of the preceding embodiments, wherein the cation is selected from the group consisting of sodium, potassium, ammonium, calcium, lithium, magnesium, aluminum, cesium, barium, straight trimethyl ammonium, branched trimethyl ammonium, triethyl ammonium, tripropyl ammonium, tributyl ammonium, tetramethyl ammonium, tetraethyl ammonium, tetrapropyl ammonium, tetrabutyl ammonium, and any combination thereof.

Embodiment 20. The method of any one of the preceding embodiments, wherein the at least one anionic surfactant is selected from the group consisting of sodium cholate, sodium N-lauroyl sarcosine (NLS), sodium dodecyl sulfate (SDS), sodium dodecyl benzene sulfonate (SDBS), and any combination thereof.

Embodiment 21. The method of any one of the preceding embodiments, wherein when the at least one non-ionic surfactant and the at least one anionic surfactant are dissolved in a volume of water, the anionic surfactant is present at a concentration of about 0.03M to 0.4M, and the non-ionic surfactant is present at a concentration of about 1-20% (v/v).

Embodiment 22. The method of embodiment 21, wherein the anionic surfactant is present at a concentration of about 0.09M to 0.095M, and the non-ionic surfactant is present at a concentration of about 10% (v/v).

Embodiment 23. The method of any one of embodiments 1-22, wherein the at least one anionic surfactant is NLS, and the at least one non-ionic surfactant is selected from the group consisting of: (i) the compound of Formula I, wherein n is 7 or 8 or a mixture thereof, and q is 8; or n is 9 or 10 or a mixture thereof, and q is 8; (ii) the compound of Formula II, wherein w+x+y+z=20; and (iii) the compound of Formula III, wherein n is 23.

Embodiment 24. The method of any one of embodiments 1-22, wherein the at least one anionic surfactant is SDS, and the at least one non-ionic surfactant is selected from the group consisting of: (i) the compound of Formula I, wherein n is 7 or 8 or a mixture thereof, and q is 8; or n is 9 or 10 or a mixture thereof, and q is 8; (ii) the compound of Formula II, wherein w+x+y+z=20; and (iii) the compound of Formula III, wherein n is 23.

Embodiment 25. The method of any one of embodiments 1-22, wherein the at least one anionic surfactant is SDBS, and the at least one non-ionic surfactant is selected from the group consisting of: (i) the compound of Formula I, wherein n is 7 or 8 or a mixture thereof, and q is 8; or n is 9 or 10 or a mixture thereof, and q is 8; (ii) the compound of Formula II, wherein w+x+y+z=20; and (iii) the compound of Formula III, wherein n is 23.

Embodiment 26. The method of any one of embodiments 1-22, wherein the at least one anionic surfactant is sodium cholate, and the at least one non-ionic surfactant is selected from the group consisting of: (i) the compound of Formula I, wherein n is 9 or 10 or a mixture thereof, and q is 8; (ii) the compound of Formula II, wherein w+x+y+Z=20; and (iii) the compound of Formula III, wherein n is 23.

Embodiment 27. The method of embodiment 23, wherein the at least one anionic surfactant is NLS, and the at least one non-ionic surfactant is the compound of Formula I, wherein n is 9 or 10 or a mixture thereof, and q is 8, and the average molecular weight of the at least one non-ionic surfactant is about 625 g/mol.

Embodiment 28. The method of embodiment 23, wherein the at least one anionic surfactant is NLS, and the at least one non-ionic surfactant is the compound of Formula I, wherein n is 9 or 10 or a mixture thereof, and q is 8, and the average molecular weight of the at least one non-ionic surfactant is about 603 g/mol.

Embodiment 29. The method of embodiment 23, wherein the at least one anionic surfactant is NLS, and the at least one non-ionic surfactant is the compound of Formula I, wherein n is 7 or 8 or a mixture thereof, and q is 8.

Embodiment 30. The method of embodiment 23, wherein the at least one anionic surfactant is NLS, and the at least one non-ionic surfactant is the compound of Formula III, wherein n is 23.

Embodiment 31. The method of embodiment 23, wherein the at least one anionic surfactant is NLS, and the at least one non-ionic surfactant is the compound of Formula II, wherein w+x+y+z=20.

Embodiment 32. The method of embodiment 24, wherein the at least one anionic surfactant is SDS, and the at least one non-ionic surfactant is Formula I, wherein n is 9 or 10 or a mixture thereof, and q is 8, and the average molecular weight of the at least one non-ionic surfactant is about 625 g/mol.

Embodiment 33. The method of embodiment 24, wherein the at least one anionic surfactant is SDS, and the at least one non-ionic surfactant is Formula I, wherein n is 9 or 10 or a mixture thereof, and q is 8, and the average molecular weight of the at least one non-ionic surfactant is about 603 g/mol.

Embodiment 34. The method of embodiment 24, wherein the at least one anionic surfactant is SDS, and the at least one non-ionic surfactant is Formula I, wherein n is 7 or 8 or a mixture thereof, and q is 8.

Embodiment 35. The method of embodiment 24, wherein the at least one anionic surfactant is SDS, and the at least one non-ionic surfactant is Formula III, wherein n is 23.

Embodiment 36. The method of embodiment 24, wherein the at least one anionic surfactant is SDS, and the at least one non-ionic surfactant is Formula II, wherein w+x+y+z=20.

Embodiment 37. The method of embodiment 25, wherein the at least one anionic surfactant is SDBS, and the at least one non-ionic surfactant is Formula I, wherein n is 9 or 10 or a mixture thereof, and q is 8, and the average molecular weight of the at least one non-ionic surfactant is about 625 g/mol.

Embodiment 38. The method of embodiment 25, wherein the at least one anionic surfactant is SDBS, and the at least one non-ionic surfactant is Formula I, wherein n is 9 or 10 or a mixture thereof, and q is 8, and the average molecular weight of the at least one non-ionic surfactant is about 603 g/mol.

Embodiment 39. The method of embodiment 25, wherein the at least one anionic surfactant is SDBS, and the at least one non-ionic surfactant is Formula I, wherein n is 7 or 8 or a mixture thereof, and q is 8.

Embodiment 40. The method of embodiment 25, wherein the at least one anionic surfactant is SDBS, and the at least one non-ionic surfactant is Formula III, wherein n is 23.

Embodiment 41. The method of embodiment 25, wherein the at least one anionic surfactant is SDBS, and the at least one non-ionic surfactant is Formula II, wherein w+x+y+z=20.

Embodiment 42. The method of embodiment 26, wherein the at least one anionic surfactant is sodium cholate, and the at least one non-ionic surfactant is Formula I, wherein n is 9 or 10 or a mixture thereof, and q is 8, and the average molecular weight of the at least one non-ionic surfactant is about 625 g/mol.

Embodiment 43. The method of embodiment 26, wherein the at least one anionic surfactant is sodium cholate, and the at least one non-ionic surfactant is Formula I, wherein n is 9 or 10 or a mixture thereof, and q is 8, and the average molecular weight of the at least one non-ionic surfactant is about 603 g/mol.

Embodiment 44. The method of embodiment 26, wherein the at least one anionic surfactant is sodium cholate, and the at least one non-ionic surfactant is Formula I, wherein n is 7 or 8 or a mixture thereof, and q is 8.

Embodiment 45. The method of embodiment 26, wherein the at least one anionic surfactant is sodium cholate, and the at least one non-ionic surfactant is Formula III, wherein n is 23.

Embodiment 46. The method of embodiment 26, wherein the at least one anionic surfactant is sodium cholate, and the at least one non-ionic surfactant is Formula II, wherein w+x+y+z=20.

Embodiment 47. The method of any one of embodiments 1-46, wherein the composition further comprises one or more of a pH buffer, salt, proteinase, or combination thereof.

Embodiment 48. The method of embodiment 47, wherein the pH buffer is Tris-HCl.

Embodiment 49. The method of embodiment 47 or 48, wherein the pH buffer has a concentration of about 0.3-3M.

Embodiment 50. The method of any one of embodiments 47-49, wherein the salt is selected from the group consisting of NaCl, KCl, NH₄Cl, Na₂SO₄, K₂SO₄, (NH₄)₂SO₄, guanidinium hydrochloride, and guanidinium thiocyanate.

Embodiment 51. The method of embodiment 47, wherein the salt is NaCl.

Embodiment 52. The method of any one of embodiments 47-51, wherein the salt has a concentration of about 0.3-3M.

Embodiment 53. The method of any one of embodiments 47-52, wherein the proteinase is selected from the group consisting of Arg-C proteinase, BNPS-Skatole, Caspase3, Caspase6, Caspase9, Chymotrypsin-high specificity, Clostripain, Factor Xa, GranzymeB, LysC, Neutrophil elastase, Pepsin, Proteinase K, Thermolysin, Asp-N endopeptidase, Caspase1, Caspase4, Caspase7, Caspase10, Chymotrypsin-low specificity, CNBr, Formic acid, Hydroxylamine, LysN, Staphylococcal peptidase I, Thrombin, Asp-N endopeptidase+N-terminal Glu, Caspase2, Caspase5, Caspase8, Enterokinase, Glutamyl endopeptidase, Iodosobenzoic acid, 2-nitro-5-thiocyanobenzoic acid, Proline-endopeptidase, Tobacco etch virus protease, and Trypsin.

Embodiment 54. The method of embodiment 53, wherein the proteinase is Proteinase K.

Embodiment 55. The method of any one of embodiments 27-31, wherein the composition further comprises NaCl present at a concentration of about 0.3-3M and Tris-HCl present at a concentration of about 0.3-3M.

Embodiment 56. The method of any one of embodiments 1-21, wherein the at least one anionic surfactant is 0.07-0.11M NLS, the at least one non-ionic surfactant is 8-12% (v/v) Formula I, wherein n is 9 or 10 or a mixture thereof, and q is 8, the average molecular weight of the at least one non-ionic surfactant is about 625 g/mol, and wherein the composition further comprises 1.2-1.5M NaCl and 2.1-2.4M Tris-HCl.

Embodiment 57. The method of any one of embodiments 1-21, wherein the at least one anionic surfactant is 0.07-0.11M NLS, the at least one non-ionic surfactant is 8-12% (v/v) Formula I, wherein n is 9 or 10 or a mixture thereof, and q is 8, the average molecular weight of the at least one non-ionic surfactant is about 603 g/mol, and wherein the composition further comprises 1.2-1.5M NaCl and 2.1-2.4M Tris-HCl.

Embodiment 58. The method of any one of embodiments 1-21, wherein the at least one anionic surfactant is 0.07-0.11M NLS, the at least one non-ionic surfactant is 8-12% (v/v) Formula I, wherein n is 7 or 8 or a mixture thereof, and q is 8, and wherein the composition further comprises 1.2-1.5M NaCl and 2.1-2.4M Tris-HCl.

Embodiment 59. The method of any one of embodiments 1-21, wherein the at least one anionic surfactant is 0.07-0.11M NLS, the at least one non-ionic surfactant is 8-12% (v/v) Formula III, wherein n is 23, and wherein the composition further comprises 1.2-1.5M NaCl and 2.1-2.4M Tris-HCl.

Embodiment 60. The method of any one of embodiments 1-21, wherein the at least one anionic surfactant is 0.07-0.11M NLS, the at least one non-ionic surfactant is 8-12% (v/v) Formula II, wherein w+x+y+z is 20, and wherein the composition further comprises 1.2-1.5M NaCl and 2.1-2.4M Tris-HCl.

Embodiment 61. The method of any one of embodiments 1-21, wherein the at least one anionic surfactant is 0.07-0.11M SDS, the at least one non-ionic surfactant is 8-12% (v/v) Formula I, wherein n is 9 or 10 or a mixture thereof, and q is 8, and the average molecular weight of the at least one non-ionic surfactant is about 625 g/mol.

Embodiment 62. The method of any one of embodiments 1-21, wherein the at least one anionic surfactant is 0.07-0.11M SDS, the at least one non-ionic surfactant is 8-12% (v/v) Formula I, wherein n is 9 or 10 or a mixture thereof, and q is 8, and the average molecular weight of the at least one non-ionic surfactant is about 603 g/mol.

Embodiment 63. The method of any one of embodiments 1-21, wherein the at least one anionic surfactant is 0.07-0.11M SDS, the at least one non-ionic surfactant is 8-12% (v/v) Formula I, wherein n is 7 or 8 or a mixture thereof, and q is 8.

Embodiment 64. The method of any one of embodiments 1-21, wherein the at least one anionic surfactant is 0.07-0.11M SDS, and the at least one non-ionic surfactant is 8-12% (v/v) Formula III, wherein n is 23.

Embodiment 65. The method of any one of embodiments 1-21, wherein the at least one anionic surfactant is 0.07-0.11M SDS, and the at least one non-ionic surfactant is 8-12% (v/v) Formula II, wherein w+x+y+z is 20.

Embodiment 66. The method of any one of embodiments 1-21, wherein the at least one anionic surfactant is 0.07-0.11M SDS, and the at least one non-ionic surfactant is 8-12% (v/v) Formula I, wherein n is 9 or 10 or a mixture thereof, and q is 8.

Embodiment 67. The method of any one of embodiments 1-21, wherein the at least one anionic surfactant is 0.07-0.11M SDBS, and the at least one non-ionic surfactant is 8-12% (v/v) Formula I, wherein n is 9 or 10 or a mixture thereof, and q is 8, and the average molecular weight of the at least one non-ionic surfactant is about 625 g/mol.

Embodiment 68. The method of any one of embodiments 1-21, wherein the at least one anionic surfactant is 0.07-0.11M SDBS, and the at least one non-ionic surfactant is 8-12% (v/v) Formula I, wherein n is 9 or 10 or a mixture thereof, and q is 8, and the average molecular weight of the at least one non-ionic surfactant is about 603 g/mol.

Embodiment 69. The method of any one of embodiments 1-21, wherein the at least one anionic surfactant is 0.07-0.11M SDBS, and the at least one non-ionic surfactant is 8-12% (v/v) Formula I, wherein n is 7 or 8 or a mixture thereof.

Embodiment 70. The method of any one of embodiments 1-21, wherein the at least one anionic surfactant is 0.07-0.11M SDBS, and the at least one non-ionic surfactant is 8-12% (v/v) Formula III, wherein n is 23.

Embodiment 71. The method of any one of embodiments 1-21, wherein the at least one anionic surfactant is 0.07-0.11M SDBS, and the at least one non-ionic surfactant is 8-12% (v/v) Formula II, wherein w+x+y+z is 20.

Embodiment 72. The method of any one of embodiments 1-21, wherein the at least one anionic surfactant is 0.07-0.11M SDBS, and the at least one non-ionic surfactant is 8-12% (v/v) Formula I, wherein n is 9 or 10 or a mixture thereof, and q is 8.

Embodiment 73. The method of any one of embodiments 1-21, wherein the at least one anionic surfactant is 0.07-0.11M sodium cholate, and the at least one non-ionic surfactant is 8-12% (v/v) Formula I, wherein n is 9 or 10 or a mixture thereof, and q is 8, and the average molecular weight of the at least one non-ionic surfactant is about 625 g/mol.

Embodiment 74. The method of any one of embodiments 1-21, wherein the at least one anionic surfactant is 0.07-0.11M sodium cholate, and the at least one non-ionic surfactant is 8-12% (v/v) Formula I, wherein n is 9 or 10 or a mixture thereof, and q is 8, and the average molecular weight of the at least one non-ionic surfactant is about 603 g/mol.

Embodiment 75. The method of any one of embodiments 1-21, wherein the at least one anionic surfactant is 0.07-0.11M sodium cholate, and the at least one non-ionic surfactant is 8-12% (v/v) Formula I, wherein n is 7 or 8 or a mixture thereof.

Embodiment 76. The method of any one of embodiments 1-21, wherein the at least one anionic surfactant is 0.07-0.11M sodium cholate, and the at least one non-ionic surfactant is 8-12% (v/v) Formula III, wherein n is 23.

Embodiment 77. The method of any one of embodiments 1-21, wherein the at least one anionic surfactant is 0.07-0.11M sodium cholate, and the at least one non-ionic surfactant is 8-12% (v/v) Formula II, wherein w+x+y+z is 20.

Embodiment 78. The method of any one of embodiments 1-21, wherein the at least one anionic surfactant is 0.07-0.11M sodium cholate, and the at least one non-ionic surfactant is 8-12% (v/v) Formula I, wherein n is 9 or 10 or a mixture thereof, and q is 8.

Embodiment 79. The method of any one of embodiments 1-21, wherein the at least one anionic surfactant is about 0.095M NLS, the at least one non-ionic surfactant is about 10% (v/v) Formula I, wherein n is 9 or 10 or a mixture thereof, and q is 8, the average molecular weight of the at least one non-ionic surfactant is about 625 g/mol, and wherein the composition further comprises about 1.35 M NaCl and about 2.25 M Tris-HCl.

Embodiment 80. The method of any one of embodiments 1-21, wherein the at least one anionic surfactant is about 0.095M NLS, the at least one non-ionic surfactant is about 10% (v/v) Formula I, wherein n is 9 or 10 or a mixture thereof, and q is 8, the average molecular weight of the at least one non-ionic surfactant is about 603 g/mol, and wherein the composition further comprises about 1.35 M NaCl and about 2.25 M Tris-HCl.

Embodiment 81. The method of any one of embodiments 1-21, wherein the at least one anionic surfactant is about 0.095M NLS, the at least one non-ionic surfactant is about 10% (v/v) Formula I, wherein n is 7 or 8 or a mixture thereof, and q is 8, and wherein the composition further comprises 1 about 1.35 M NaCl and about 2.25 M Tris-HCl.

Embodiment 82. The method of any one of embodiments 1-21, wherein the at least one anionic surfactant is about 0.095M NLS, the at least one non-ionic surfactant is about 10% (v/v) Formula III, wherein n is 23, and wherein the composition further about 1.35 M NaCl and about 2.25 M Tris-HCl.

Embodiment 83. The method of any one of embodiments 1-21, wherein the at least one anionic surfactant is about 0.095M NLS, the at least one non-ionic surfactant is about 10% (v/v) Formula II, wherein w+x+y+z is 20, and wherein the composition further comprises about 1.35 M NaCl and about 2.25 M Tris-HCl.

Embodiment 84. The method of any one of embodiments 1-21, wherein the at least one anionic surfactant is about 0.09M SDS, the at least one non-ionic surfactant is about 10% (v/v) Formula I, wherein n is 9 or 10 or a mixture thereof, and q is 8, and the average molecular weight of the at least one non-ionic surfactant is about 625 g/mol.

Embodiment 85. The method of any one of embodiments 1-21, wherein the at least one anionic surfactant is about 0.09M SDS, the at least one non-ionic surfactant is about 10% (v/v) Formula I, wherein n is 9 or 10 or a mixture thereof, and q is 8, and the average molecular weight of the at least one non-ionic surfactant is about 603 g/mol.

Embodiment 86. The method of any one of embodiments 1-21, wherein the at least one anionic surfactant is about 0.09M SDS, and the at least one non-ionic surfactant is about 10% (v/v) Formula I, wherein n is 7 or 8 or a mixture thereof, and q is 8.

Embodiment 87. The method of any one of embodiments 1-21, wherein the at least one anionic surfactant is about 0.09M SDS, and the at least one non-ionic surfactant is about 10% (v/v) Formula III, wherein n is 23.

Embodiment 88. The method of any one of embodiments 1-21, wherein the at least one anionic surfactant is about 0.09M SDS, and the at least one non-ionic surfactant is about 10% (v/v) Formula II, wherein w+x+y+z is 20.

Embodiment 89. The method of any one of embodiments 1-21, wherein the at least one anionic surfactant is about 0.09M SDS, and the at least one non-ionic surfactant is about 10% (v/v) Formula I, wherein n is 9 or 10 or a mixture thereof, and q is 8.

Embodiment 90. The method of any one of embodiments 1-21, wherein the at least one anionic surfactant is about 0.09M SDBS, the at least one non-ionic surfactant is about 10% (v/v) Formula I, wherein n is 9 or 10 or a mixture thereof, and q is 8, and the average molecular weight of the at least one non-ionic surfactant is about 625 g/mol.

Embodiment 91. The method of any one of embodiments 1-21, wherein the at least one anionic surfactant is about 0.09M SDBS, the at least one non-ionic surfactant is about 10% (v/v) Formula I, wherein n is 9 or 10 or a mixture thereof, and q is 8, and the average molecular weight of the at least one non-ionic surfactant is about 603 g/mol.

Embodiment 92. The method of any one of embodiments 1-21, wherein the at least one anionic surfactant is about 0.09M SDBS, and the at least one non-ionic surfactant is about 10% (v/v) Formula I, wherein n is 7 or 8 or a mixture thereof, and q is 8.

Embodiment 93. The method of any one of embodiments 1-21, wherein the at least one anionic surfactant is about 0.09M SDBS, and the at least one non-ionic surfactant is about 10% (v/v) Formula III, wherein n is 23.

Embodiment 94. The method of any one of embodiments 1-21, wherein the at least one anionic surfactant is about 0.09M SDBS, and the at least one non-ionic surfactant is about 10% (v/v) Formula II, wherein w+x+y+z is 20.

Embodiment 95. The method of any one of embodiments 1-21, wherein the at least one anionic surfactant is about 0.09M SDBS, and the at least one non-ionic surfactant is about 10% (v/v) Formula I, wherein n is 9 or 10 or a mixture thereof, and q is 8.

Embodiment 96. The method of any one of embodiments 1-21, wherein the at least one anionic surfactant is about 0.09M sodium cholate, the at least one non-ionic surfactant is about 10% (v/v) Formula I, wherein n is 9 or 10 or a mixture thereof, and q is 8, and the average molecular weight of the at least one non-ionic surfactant is about 625 g/mol.

Embodiment 97. The method of any one of embodiments 1-21, wherein the at least one anionic surfactant is about 0.09M sodium cholate, the at least one non-ionic surfactant is about 10% (v/v) Formula I, wherein n is 9 or 10 or a mixture thereof, and q is 8, and the average molecular weight of the at least one non-ionic surfactant is about 603 g/mol.

Embodiment 98. The method of any one of embodiments 1-21, wherein the at least one anionic surfactant is about 0.09M sodium cholate, and the at least one non-ionic surfactant is about 10% (v/v) Formula I, wherein n is 7 or 8 or a mixture thereof, and q is 8.

Embodiment 99. The method of any one of embodiments 1-21, wherein the at least one anionic surfactant is about 0.09M sodium cholate, and the at least one non-ionic surfactant is about 10% (v/v) Formula III, wherein n is 23.

Embodiment 100. The method of any one of embodiments 1-21, wherein the at least one anionic surfactant is about 0.09M sodium cholate, and the at least one non-ionic surfactant is about 10% (v/v) Formula II, wherein w+x+y+z is 20.

Embodiment 101. The method of any one of embodiments 1-21, wherein the at least one anionic surfactant is about 0.09M sodium cholate, and the at least one non-ionic surfactant is about 10% (v/v) Formula I, wherein n is 9 or 10 or a mixture thereof, and q is 8.

Embodiment 102. The method of any one of the preceding embodiments, wherein the nucleic acid components are DNA, RNA, or any combination thereof.

Embodiment 103. The method of embodiment 102, wherein the nucleic acid components are genomic DNA, cDNA, plasmid DNA, cell-free DNA, circulating tumor DNA, micro RNA, messenger RNA, transfer RNA, ribosomal RNA, long non-coding RNA, siRNA, or any combination thereof.

Embodiment 104. The method of embodiment 102, wherein the nucleic acid components are cell-free DNA.

Embodiment 105. The method of any one of the preceding embodiments, wherein the binding buffer comprises a chaotropic agent selected from the group consisting of guanidinium hydrochloride (GHCl), guanidinium thiocyanate, guanidinium isothiocyanate (GITC)), sodium thiocyanate, sodium iodide, sodium perchlorate, sodium trichloroacetate, sodium trifluoroacetate, lithium perchlorate, lithium acetate, magnesium chloride, phenol, 2-propanol, thiourea, and urea.

Embodiment 106. The method of embodiment 105, wherein the chaotropic agent is present at a concentration of 2M to 7M.

Embodiment 107. The method of embodiment 105 or 106, wherein the binding buffer further comprises a polymer at a concentration of 5-20% (w/v).

Embodiment 108. The method of any one of the preceding embodiments, wherein the binding buffer comprises guanidine thiocyanate and PEG, and the purification system is magnetic beads.

Embodiment 109. The method of any one of the preceding embodiments, wherein the biological sample is selected from the group consisting of blood, plasma, urine, saliva, stool, cerebrospinal fluid (CSF), lymph, serum, sputum, peritoneal fluid, sweat, tears, nasal swab, vaginal swab, endocervical swab, semen, or breast milk.

Embodiment 110. The method of any one of the preceding embodiments, wherein the composition consists essentially of one non-ionic surfactant and one anionic surfactant.

Embodiment 111. A lysing composition, comprising: (a) at least one non-ionic surfactant with a hydrophilic-lipophilic balance (HLB) value greater than or equal to 10; and (b) at least one anionic surfactant, wherein the anionic surfactant comprises a cation and an anionic group; wherein the anionic group is selected from the group consisting of a cholate, a carboxylate, a sulfonate, a sulfate, and a phosphate ester.

Embodiment 112. The composition of embodiment 111, wherein the hydrophilic-lipophilic balance (HLB) value of the at least one non-ionic surfactant is between 10 and 20.

Embodiment 113. The composition of embodiment 111, wherein the hydrophilic-lipophilic balance (HLB) value of the at least one non-ionic surfactant is between 10 and 17.

Embodiment 114. The composition of embodiment 111, wherein the hydrophilic-lipophilic balance (HLB) value of the at least one non-ionic surfactant is between 12.4 and 16.9.

Embodiment 115. The composition of embodiment 111, wherein the hydrophilic-lipophilic balance (HLB) value of the at least one non-ionic surfactant is 12.4, 13.4, 16.7 or 16.9.

Embodiment 116. The composition of any one of embodiments 111-115, wherein the at least one non-ionic surfactant comprises at least one —(OCH₂CH₂)ₙ— chain and at least one OH group, wherein n is a whole number between 2 and 40.

Embodiment 117. The composition of embodiment 116, wherein the at least one non-ionic surfactant is selected from the group consisting of ethoxylated natural fatty alcohol, octylphenoxy alcohol, ethoxylated sorbitan ester, and any combination thereof.

Embodiment 118. The composition of embodiment 117, wherein the ethoxylated natural fatty alcohol is a compound of Formula III: CH₃(CH₂)₁₀CH₂(OCH₂CH₂)ₙOH (Formula III), wherein n is 10-23.

Embodiment 119. The composition of embodiment 117, wherein the octylphenoxy alcohol comprises one or more compounds of Formula I:

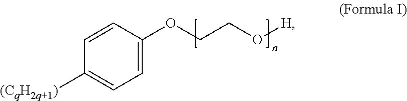

wherein n is 6, 7, 8, 9, or 10, and q is 6, 7, 8, 9, 10, 11, or 12.

Embodiment 120. The composition of embodiment 119, wherein n is 7 or 8 or a mixture thereof; or 9 or 10 or a mixture thereof.

Embodiment 121. The composition of embodiment 119, wherein n is 9, 10, or a mixture thereof; and the average molecular weight of the octylphenoxy alcohol is about 603 g/mol or about 625 g/mol.

Embodiment 122. The composition of embodiment 117, wherein the ethoxylated sorbitan ester is a compound of Formula II:

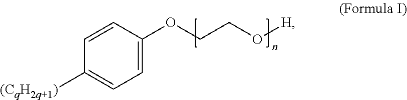

wherein w+x+y+z=20.

Embodiment 123. The composition of embodiment 111, wherein the at least one non-ionic surfactant is selected from the group consisting of Triton X-100, Triton X-114, Brij L23, Tween 20 and IGEPAL CA630.

Embodiment 124. The composition of any one of embodiments 111-123, wherein the sulfonate is selected from the group consisting of petroleum sulfonate, alkylbenzenesulfonate, naphthalenesulfonate, olefin sulfonate, and any combination thereof; and wherein the sulfate is selected from the group consisting of alkyl sulfate, sulfated natural oil, sulfated natural fat, sulfated ester, sulfated alkanolamide, sulfated alkylphenol, and any combination thereof.

Embodiment 125. The composition of any one of embodiments 111-124, wherein the cholate is sodium cholate, the carboxylate is sodium N-lauroyl sarcosine (NLS), the sulfate is sodium dodecyl sulfate (SDS), and the sulfonate is sodium dodecyl benzene sulfonate (SDBS).

Embodiment 126. The composition of any one of embodiments 111-125, wherein the cation is selected from the group consisting of sodium, potassium, ammonium, calcium, lithium, magnesium, aluminium, cesium, barium, straight trimethyl ammonium, branched trimethyl ammonium, triethyl ammonium, tripropyl ammonium, tributyl ammonium, tetramethyl ammonium, tetraethyl ammonium, tetrapropyl ammonium, tetrabutyl ammonium, and any combination thereof.

Embodiment 127. The composition of any one of embodiments 111-126, wherein the at least one anionic surfactant is selected from the group consisting of sodium cholate, sodium N-lauroyl sarcosine (NLS), sodium dodecyl sulfate (SDS), sodium dodecyl benzene sulfonate (SDBS), and any combination thereof.

Embodiment 128. The composition of any one of embodiments 111-127, wherein when the at least one non-ionic surfactant and the at least one anionic surfactant are dissolved in a volume of water, the anionic surfactant is present at a concentration of about 0.03M to 0.4M, and the non-ionic surfactant is present at a concentration of about 1-20% (v/v).

Embodiment 129. The composition of embodiment 128, wherein the anionic surfactant is present at a concentration of about 0.09M to 0.095M, and the non-ionic surfactant is present at a concentration of about 10% (v/v).

Embodiment 130. The composition of any one of embodiments 111-129, wherein the at least one anionic surfactant is NLS, and the at least one non-ionic surfactant is selected from the group consisting of: (i) the compound of Formula I, wherein n is 7 or 8 or a mixture thereof, and q is 8; or n is 9 or 10 or a mixture thereof, and q is 8; (ii) the compound of Formula II, wherein w+x+y+z=20; and (iii) the compound of Formula III, wherein n is 23.

Embodiment 131. The composition of any one of embodiments 111-129, wherein the at least one anionic surfactant is SDS, and the at least one non-ionic surfactant is selected from the group consisting of: (i) the compound of Formula I, wherein n is 7 or 8 or a mixture thereof, and q is 8; or n is 9 or 10 or a mixture thereof, and q is 8; (ii) the compound of Formula II, wherein w+x+y+z=20; and (iii) the compound of Formula III, wherein n is 23.

Embodiment 132. The composition of any one of embodiments 111-129, wherein the at least one anionic surfactant is SDBS, and the at least one non-ionic surfactant is selected from the group consisting of: (i) the compound of Formula I, wherein n is 7 or 8 or a mixture thereof, and q is 8; or n is 9 or 10 or a mixture thereof, and q is 8; (ii) the compound of Formula II, wherein w+x+y+z=20; and (iii) the compound of Formula III, wherein n is 23.

Embodiment 133. The composition of any one of embodiments 111-129, wherein the at least one anionic surfactant is sodium cholate, and the at least one non-ionic surfactant is selected from the group consisting of: (i) the compound of Formula I, wherein n is 9 or 10 or a mixture thereof, and q is 8; (ii) the compound of Formula II, wherein w+x+y+z=20; and (iii) the compound of Formula III, wherein n is 23.

Embodiment 134. The composition of embodiment 130, wherein the at least one anionic surfactant is NLS, and the at least one non-ionic surfactant is Formula I, wherein n is 9 or 10 or a mixture thereof, and q is 8, and the average molecular weight of the at least one non-ionic surfactant is about 625 g/mol.

Embodiment 135. The composition of embodiment 130, wherein the at least one anionic surfactant is NLS, and the at least one non-ionic surfactant is Formula I, wherein n is 9 or 10 or a mixture thereof, and q is 8, and the average molecular weight of the at least one non-ionic surfactant is about 603 g/mol.

Embodiment 136. The composition of embodiment 130, wherein the at least one anionic surfactant is NLS, and the at least one non-ionic surfactant is Formula I, wherein n is 7 or 8 or a mixture thereof, and q is 8.

Embodiment 137. The composition of embodiment 130, wherein the at least one anionic surfactant is NLS, and the at least one non-ionic surfactant is Formula III, wherein n is 23.

Embodiment 138. The composition of embodiment 130, wherein the at least one anionic surfactant is NLS, and the at least one non-ionic surfactant is Formula II, wherein w+x+y+z=20.

Embodiment 139. The composition of embodiment 131, wherein the at least one anionic surfactant is SDS, and the at least one non-ionic surfactant is Formula I, wherein n is 9 or 10 or a mixture thereof, and q is 8, and the average molecular weight of the at least one non-ionic surfactant is about 625 g/mol.

Embodiment 140. The composition of embodiment 131, wherein the at least one anionic surfactant is SDS, and the at least one non-ionic surfactant is Formula I, wherein n is 9 or 10 or a mixture thereof, and q is 8, and the average molecular weight of the at least one non-ionic surfactant is about 603 g/mol.

Embodiment 141. The composition of embodiment 131, wherein the at least one anionic surfactant is SDS, and the at least one non-ionic surfactant is Formula I, wherein n is 7 or 8 or a mixture thereof, and q is 8.

Embodiment 142. The composition of embodiment 131, wherein the at least one anionic surfactant is SDS, and the at least one non-ionic surfactant is Formula III, wherein n is 23.

Embodiment 143. The composition of embodiment 131, wherein the at least one anionic surfactant is SDS, and the at least one non-ionic surfactant is Formula II, wherein w+x+y+z=20.

Embodiment 144. The composition of embodiment 132, wherein the at least one anionic surfactant is SDBS, the at least one non-ionic surfactant is Formula I, wherein n is 9 or 10 or a mixture thereof, and q is 8, and the average molecular weight of the at least one non-ionic surfactant is about 625 g/mol.

Embodiment 145. The composition of embodiment 132, wherein the at least one anionic surfactant is SDBS, the at least one non-ionic surfactant is Formula I, wherein n is 9 or 10 or a mixture thereof, and q is 8, and the average molecular weight of the at least one non-ionic surfactant is about 603 g/mol.

Embodiment 146. The composition of embodiment 132, wherein the at least one anionic surfactant is SDBS, and the at least one non-ionic surfactant is Formula I, wherein n is 7 or 8 or a mixture thereof, and q is 8.

Embodiment 147. The composition of embodiment 132, wherein the at least one anionic surfactant is SDBS, and the at least one non-ionic surfactant is Formula III, wherein n is 23.

Embodiment 148. The composition of embodiment 132, wherein the at least one anionic surfactant is SDBS, and the at least one non-ionic surfactant is Formula II, wherein w+x+y+z=20.

Embodiment 149. The composition of embodiment 133, wherein the at least one anionic surfactant is sodium cholate, the at least one non-ionic surfactant is Formula I, wherein n is 9 or 10 or a mixture thereof, and q is 8, and the average molecular weight of the at least one non-ionic surfactant is about 625 g/mol.

Embodiment 150. The composition of embodiment 133, wherein the at least one anionic surfactant is sodium cholate, the at least one non-ionic surfactant is Formula I, wherein n is 9 or 10 or a mixture thereof, and q is 8, and the average molecular weight of the at least one non-ionic surfactant is about 603 g/mol.

Embodiment 151. The composition of embodiment 133, wherein the at least one anionic surfactant is sodium cholate, and the at least one non-ionic surfactant is Formula I, wherein n is 7 or 8 or a mixture thereof, and q is 8.

Embodiment 152. The composition of embodiment 133, wherein the at least one anionic surfactant is sodium cholate, and the at least one non-ionic surfactant is Formula III, wherein n is 23.

Embodiment 153. The composition of embodiment 133, wherein the at least one anionic surfactant is sodium cholate, and the at least one non-ionic surfactant is Formula II, wherein w+x+y+z=20.

Embodiment 154. The composition of any one of embodiments 111-153, further comprising one or more of a pH buffer, salt, proteinase, or combination thereof.

Embodiment 155. The composition of embodiment 154, wherein the pH buffer is Tris-HCl.

Embodiment 156. The composition of embodiment 154 or 155, wherein the pH buffer has a concentration of about 0.3-3M.

Embodiment 157. The composition of any one of embodiments 154-156, wherein the salt is selected from the group consisting of NaCl, KCl, NH₄Cl, Na₂SO₄, K₂SO₄, (NH₄)₂SO₄, guanidinium hydrochloride, and guanidinium thiocyanate.

Embodiment 158. The composition of embodiment 154, wherein the salt is NaCl.

Embodiment 159. The composition of any one of embodiments 154-158, wherein the salt has a concentration of about 0.3-3M.

Embodiment 160. The composition of any one of embodiments 154-159, wherein the proteinase is selected from the group consisting of Arg-C proteinase, BNPS-Skatole, Caspase3, Caspase6, Caspase9, Chymotrypsin-high specificity, Clostripain, Factor Xa, GranzymeB, LysC, Neutrophil elastase, Pepsin, Proteinase K, Thermolysin, Asp-N endopeptidase, Caspase1, Caspase4, Caspase7, Caspase10, Chymotrypsin-low specificity, CNBr, Formic acid, Hydroxylamine, LysN, Staphylococcal peptidase I, Thrombin, Asp-N endopeptidase+N-terminal Glu, Caspase2, Caspase5, Caspase8, Enterokinase, Glutamyl endopeptidase, Iodosobenzoic acid, 2-nitro-5-thiocyanobenzoic acid, Proline-endopeptidase, Tobacco etch virus protease, and Trypsin.

Embodiment 161. The composition of embodiment 160, wherein the proteinase is Proteinase K.

Embodiment 162. The composition of any one of embodiments 134-138, wherein the composition further comprises NaCl present at a concentration of about 0.3-3M and Tris-HCl present at a concentration of about 0.3-3M.

Embodiment 163. The composition of any one of embodiments 111-128, wherein the at least one anionic surfactant is 0.07-0.11M NLS, the at least one non-ionic surfactant is 8-12% (v/v) Formula I, wherein n is 9 or 10 or a mixture thereof, and q is 8, the average molecular weight of the at least one non-ionic surfactant is about 625 g/mol, and wherein the composition further comprises 1.2-1.5M NaCl and 2.1-2.4M Tris-HCl.

Embodiment 164. The composition of any one of embodiments 111-128, wherein the at least one anionic surfactant is 0.07-0.11M NLS, the at least one non-ionic surfactant is 8-12% (v/v) Formula I, wherein n is 9 or 10 or a mixture thereof, and q is 8, the average molecular weight of the at least one non-ionic surfactant is about 603 g/mol, and wherein the composition further comprises 1.2-1.5M NaCl and 2.1-2.4M Tris-HCl.

Embodiment 165. The composition of any one of embodiments 111-128, wherein the at least one anionic surfactant is 0.07-0.11M NLS, the at least one non-ionic surfactant is 8-12% (v/v) Formula I, wherein n is 7 or 8 or a mixture thereof, and q is 8, and wherein the composition further comprises 1.2-1.5M NaCl and 2.1-2.4M Tris-HCl.

Embodiment 166. The composition of any one of embodiments 111-128, wherein the at least one anionic surfactant is 0.07-0.11M NLS, the at least one non-ionic surfactant is 8-12% (v/v) Formula III, wherein n is 23, and wherein the composition further comprises 1.2-1.5M NaCl and 2.1-2.4M Tris-HCl.

Embodiment 167. The composition of any one of embodiments 111-128, wherein the at least one anionic surfactant is 0.07-0.11M NLS, the at least one non-ionic surfactant is 8-12% (v/v) Formula II, wherein w+x+y+z is 20, and wherein the composition further comprises 1.2-1.5M NaCl and 2.1-2.4M Tris-HCl.

Embodiment 168. The composition of any one of embodiments 111-128, wherein the at least one anionic surfactant is 0.07-0.11M SDS, the at least one non-ionic surfactant is 8-12% (v/v) Formula I, wherein n is 9 or 10 or a mixture thereof, and q is 8, and the average molecular weight of the at least one non-ionic surfactant is about 625 g/mol.

Embodiment 169. The composition of any one of embodiments 111-128, wherein the at least one anionic surfactant is 0.07-0.11M SDS, the at least one non-ionic surfactant is 8-12% (v/v) Formula I, wherein n is 9 or 10 or a mixture thereof, and q is 8, and the average molecular weight of the at least one non-ionic surfactant is about 603 g/mol.

Embodiment 170. The composition of any one of embodiments 111-128, wherein the at least one anionic surfactant is 0.07-0.11M SDS, and the at least one non-ionic surfactant is 8-12% (v/v) Formula I, wherein n is 7 or 8 or a mixture thereof, and q is 8.

Embodiment 171. The composition of any one of embodiments 111-128, wherein the at least one anionic surfactant is 0.07-0.11M SDS, and the at least one non-ionic surfactant is 8-12% (v/v) Formula III, wherein n is 23.

Embodiment 172. The composition of any one of embodiments 111-128, wherein the at least one anionic surfactant is 0.07-0.11M SDS, and the at least one non-ionic surfactant is 8-12% (v/v) Formula II, wherein w+x+y+z is 20.

Embodiment 173. The composition of any one of embodiments 111-128, wherein the at least one anionic surfactant is 0.07-0.11M SDS, and the at least one non-ionic surfactant is 8-12% (v/v) Formula I, wherein n is 9 or 10 or a mixture thereof, and q is 8.

Embodiment 174. The composition of any one of embodiments 111-128, wherein the at least one anionic surfactant is 0.07-0.11M SDBS, the at least one non-ionic surfactant is 8-12% (v/v) Formula I, wherein n is 9 or 10 or a mixture thereof, and q is 8, and the average molecular weight of the at least one non-ionic surfactant is about 625 g/mol.

Embodiment 175. The composition of any one of embodiments 111-128, wherein the at least one anionic surfactant is 0.07-0.11M SDBS, the at least one non-ionic surfactant is 8-12% (v/v) Formula I, wherein n is 9 or 10 or a mixture thereof, and q is 8, and the average molecular weight of the at least one non-ionic surfactant is about 603 g/mol.

Embodiment 176. The composition of any one of embodiments 111-128, wherein the at least one anionic surfactant is 0.07-0.11M SDBS, and the at least one non-ionic surfactant is 8-12% (v/v) Formula I, wherein n is 7 or 8 or a mixture thereof, and q is 8.

Embodiment 177. The composition of any one of embodiments 111-128, wherein the at least one anionic surfactant is 0.07-0.11M SDBS, and the at least one non-ionic surfactant is 8-12% (v/v) Formula III, wherein n is 23.

Embodiment 178. The composition of any one of embodiments 111-128, wherein the at least one anionic surfactant is 0.07-0.11M SDBS, and the at least one non-ionic surfactant is 8-12% (v/v) Formula II, wherein w+x+y+z is 20.

Embodiment 179. The composition of any one of embodiments 111-128, wherein the at least one anionic surfactant is 0.07-0.11M SDBS, and the at least one non-ionic surfactant is 8-12% (v/v) Formula I, wherein n is 9 or 10 or a mixture thereof, and q is 8.

Embodiment 180. The composition of any one of embodiments 111-128, wherein the at least one anionic surfactant is 0.07-0.11M sodium cholate, the at least one non-ionic surfactant is 8-12% (v/v) Formula I, wherein n is 9 or 10 or a mixture thereof, and q is 8, and the average molecular weight of the at least one non-ionic surfactant is about 625 g/mol.

Embodiment 181. The composition of any one of embodiments 111-128, wherein the at least one anionic surfactant is 0.07-0.11M sodium cholate, the at least one non-ionic surfactant is 8-12% (v/v) Formula I, wherein n is 9 or 10 or a mixture thereof, and q is 8, and the average molecular weight of the at least one non-ionic surfactant is about 603 g/mol.

Embodiment 182. The composition of any one of embodiments 111-128, wherein the at least one anionic surfactant is 0.07-0.11M sodium cholate, and the at least one non-ionic surfactant is 8-12% (v/v) Formula I, wherein n is 7 or 8 or a mixture thereof, and q is 8.

Embodiment 183. The composition of any one of embodiments 111-128, wherein the at least one anionic surfactant is 0.07-0.11M sodium cholate, and the at least one non-ionic surfactant is 8-12% (v/v) Formula III, wherein n is 23.

Embodiment 184. The composition of any one of embodiments 111-128, wherein the at least one anionic surfactant is 0.07-0.11M sodium cholate, and the at least one non-ionic surfactant is 8-12% (v/v) Formula II, wherein w+x+y+z is 20.

Embodiment 185. The composition of any one of embodiments 111-128, wherein the at least one anionic surfactant is 0.07-0.11M sodium cholate, and the at least one non-ionic surfactant is 8-12% (v/v) Formula I, wherein n is 9 or 10 or a mixture thereof, and q is 8.

Embodiment 186. The composition of any one of embodiments 111-128, wherein the at least one anionic surfactant is about 0.095M NLS, the at least one non-ionic surfactant is about 10% (v/v) Formula I, wherein n is 9 or 10 or a mixture thereof, and q is 8, the average molecular weight of the at least one non-ionic surfactant is about 625 g/mol, and wherein the composition further comprises about 1.35 M NaCl and about 2.25 M Tris-HCl.

Embodiment 187. The composition of any one of embodiments 111-128, wherein the at least one anionic surfactant is about 0.095M NLS, the at least one non-ionic surfactant is about 10% (v/v) Formula I, wherein n is 9 or 10 or a mixture thereof, and q is 8, the average molecular weight of the at least one non-ionic surfactant is about 603 g/mol, and wherein the composition further comprises about 1.35 M NaCl and about 2.25 M Tris-HCl.

Embodiment 188. The composition of any one of embodiments 111-128, wherein the at least one anionic surfactant is about 0.095M NLS, the at least one non-ionic surfactant is about 10% (v/v) Formula I, wherein n is 7 or 8 or a mixture thereof, and q is 8, and wherein the composition further comprises 1 about 1.35 M NaCl and about 2.25 M Tris-HCl.

Embodiment 189. The composition of any one of embodiments 111-128, wherein the at least one anionic surfactant is about 0.095M NLS, the at least one non-ionic surfactant is about 10% (v/v) Formula III, wherein n is 23, and wherein the composition further about 1.35 M NaCl and about 2.25 M Tris-HCl.

Embodiment 190. The composition of any one of embodiments 111-128, wherein the at least one anionic surfactant is about 0.095M NLS, the at least one non-ionic surfactant is about 10% (v/v) Formula II, wherein w+x+y+z is 20, and wherein the composition further comprises about 1.35 M NaCl and about 2.25 M Tris-HCl.

Embodiment 191. The composition of any one of embodiments 111-128, wherein the at least one anionic surfactant is about 0.09M SDS, the at least one non-ionic surfactant is about 10% (v/v) Formula I, wherein n is 9 or 10 or a mixture thereof, and q is 8, and the average molecular weight of the at least one non-ionic surfactant is about 625 g/mol.

Embodiment 192. The composition of any one of embodiments 111-128, wherein the at least one anionic surfactant is about 0.09M SDS, the at least one non-ionic surfactant is about 10% (v/v) Formula I, wherein n is 9 or 10 or a mixture thereof, and q is 8, and the average molecular weight of the at least one non-ionic surfactant is about 603 g/mol.

Embodiment 193. The composition of any one of embodiments 111-128, wherein the at least one anionic surfactant is about 0.09M SDS, and the at least one non-ionic surfactant is about 10% (v/v) Formula I, wherein n is 7 or 8 or a mixture thereof.

Embodiment 194. The composition of any one of embodiments 111-128, wherein the at least one anionic surfactant is about 0.09M SDS, and the at least one non-ionic surfactant is about 10% (v/v) Formula III, wherein n is 23.

Embodiment 195. The composition of any one of embodiments 111-128, wherein the at least one anionic surfactant is about 0.09M SDS, and the at least one non-ionic surfactant is about 10% (v/v) Formula II, wherein w+x+y+z is 20.

Embodiment 196. The composition of any one of embodiments 111-128, wherein the at least one anionic surfactant is about 0.09M SDS, and the at least one non-ionic surfactant is about 10% (v/v) Formula I, wherein n is 9 or 10 or a mixture thereof, and q is 8.

Embodiment 197. The composition of any one of embodiments 111-128, wherein the at least one anionic surfactant is about 0.09M SDBS, the at least one non-ionic surfactant is about 10% (v/v) Formula I, wherein n is 9 or 10 or a mixture thereof, and q is 8, and the average molecular weight of the at least one non-ionic surfactant is about 625 g/mol.

Embodiment 198. The composition of any one of embodiments 111-128, wherein the at least one anionic surfactant is about 0.09M SDBS, the at least one non-ionic surfactant is about 10% (v/v) Formula I, wherein n is 9 or 10 or a mixture thereof, and q is 8, and the average molecular weight of the at least one non-ionic surfactant is about 603 g/mol.

Embodiment 199. The composition of any one of embodiments 111-128, wherein the at least one anionic surfactant is about 0.09M SDBS, and the at least one non-ionic surfactant is about 10% (v/v) Formula I, wherein n is 7 or 8 or a mixture thereof, and q is 8.

Embodiment 200. The composition of any one of embodiments 111-128, wherein the at least one anionic surfactant is about 0.09M SDBS, and the at least one non-ionic surfactant is about 10% (v/v) Formula III, wherein n is 23.

Embodiment 201. The composition of any one of embodiments 111-128, wherein the at least one anionic surfactant is about 0.09M SDBS, and the at least one non-ionic surfactant is about 10% (v/v) Formula II, wherein w+x+y+z is 20.

Embodiment 202. The composition of any one of embodiments 111-128, wherein the at least one anionic surfactant is about 0.09M SDBS, and the at least one non-ionic surfactant is about 10% (v/v) Formula I, wherein n is 9 or 10 or a mixture thereof, and q is 8.

Embodiment 203. The composition of any one of embodiments 111-128, wherein the at least one anionic surfactant is about 0.09M sodium cholate, the at least one non-ionic surfactant is about 10% (v/v) Formula I, wherein n is 9 or 10 or a mixture thereof, and q is 8, and the average molecular weight of the at least one non-ionic surfactant is about 625 g/mol.

Embodiment 204. The composition of any one of embodiments 111-128, wherein the at least one anionic surfactant is about 0.09M sodium cholate, the at least one non-ionic surfactant is about 10% (v/v) Formula I, wherein n is 9 or 10 or a mixture thereof, and q is 8, and the average molecular weight of the at least one non-ionic surfactant is about 603 g/mol.

Embodiment 205. The composition of any one of embodiments 111-128, wherein the at least one anionic surfactant is about 0.09M sodium cholate, and the at least one non-ionic surfactant is about 10% (v/v) Formula I, wherein n is 7 or 8 or a mixture thereof, and q is 8.

Embodiment 206. The composition of any one of embodiments 111-128, wherein the at least one anionic surfactant is about 0.09M sodium cholate, and the at least one non-ionic surfactant is about 10% (v/v) Formula III, wherein n is 23.

Embodiment 207. The composition of any one of embodiments 111-128, wherein the at least one anionic surfactant is about 0.09M sodium cholate, and the at least one non-ionic surfactant is about 10% (v/v) Formula II, wherein w+x+y+z is 20.

Embodiment 208. The composition of any one of embodiments 111-128, wherein the at least one anionic surfactant is about 0.09M sodium cholate, and the at least one non-ionic surfactant is about 10% (v/v) Formula I, wherein n is 9 or 10 or a mixture thereof, and q is 8.

Embodiment 209. A kit comprising: (a) the composition of any one of embodiments 111-208; and (b) at least one aqueous two-phase system (ATPS) composition, wherein the at least one aqueous two-phase system (ATPS) comprises a polymer, a salt, a surfactant, or any combination thereof.

What is claimed is:

1. A method for preventing debris layer formation during phase separation in an ATPS-sample lysate mixture, comprising the steps of:
(a) mixing and incubating a biological sample comprising nucleic acid components and proteins with a lysing composition to form a sample lysate, wherein the lysing composition comprises:
(i) at least one non-ionic surfactant with a hydrophilic-lipophilic balance (HLB) value between 10 and 20; and
(ii) at least one anionic surfactant, wherein the anionic surfactant comprises a cation and an anionic group; wherein the anionic group is selected from the group consisting of a cholate, a carboxylate, a sulfonate, a sulfate, and a phosphate ester; and the cation is selected from the group consisting of sodium, potassium, ammonium, calcium, lithium, magnesium, aluminum, cesium, barium, straight trimethyl ammonium, branched trimethyl ammonium, triethyl ammonium, tripropyl ammonium, tributyl ammonium, tetramethyl ammonium, tetraethyl ammonium, tetrapropyl ammonium, and tetrabutyl ammonium;
wherein the at least one non-ionic surfactant and the at least one anionic surfactant are present in a ratio such that when the at least one non-ionic surfactant and the at least one anionic surfactant are dissolved in a volume of water, the anionic surfactant is present at a concentration of about 0.5-20% (w/v), and the non-ionic surfactant is present at a concentration of about 0.5-20% (w/v), and
wherein the proteins are substantially digested in the sample lysate;
(b) adding the sample lysate to an aqueous two-phase system (ATPS) composition comprising a polymer, a salt, a surfactant, or any combination thereof dissolved in an aqueous solution to form the ATPS-sample lysate mixture, wherein the ATPS-sample lysate mixture separates into a target-rich phase solution and a target-poor phase solution such that the nucleic acid components partition selectively to the target-rich phase solution without substantial debris layer formation.

2. The method of claim 1, further comprising the steps of
(c) optionally collecting the first target-rich phase solution and mixing the first target-rich phase solution with a second ATPS composition comprising a polymer, a salt, a surfactant, or any combination thereof dissolved in an aqueous solution to form a second target-rich phase solution and a second target-poor phase solution, such that the nucleic acid components partition to the second target-rich phase solution without substantial debris layer formation; and
(d) isolating the nucleic acid components from the target-rich phase solution and/or the second target-rich phase solution.

3. The method of claim 2, wherein step (d) further comprises the following steps:
mixing the target-rich phase solution or the second target-rich phase solution with a binding buffer to form a mixed solution;
loading the mixed solution onto a purification system configured to selectively extract and purify the nucleic acid components; and
eluting and collecting the nucleic acid components from the purification system, resulting in a final solution containing the concentrated and purified nucleic acid components, wherein the purification system is a solid phase extraction column, liquid chromatography column, or magnetic beads.

4. The method of claim 1, wherein the hydrophilic-lipophilic balance (HLB) value of the at least one non-ionic surfactant is between 10 and 17.

5. The method of claim 1, wherein the at least one non-ionic surfactant comprises at least one —(OCH₂CH₂)ₙ— chain and at least one OH group, wherein n is a whole number between 2 and 40.

6. The method of claim 5, wherein the at least one non-ionic surfactant is selected from the group consisting of ethoxylated natural fatty alcohol, octylphenoxy alcohol, ethoxylated sorbitan ester, and any combination thereof.

7. The method of claim 6, wherein the ethoxylated natural fatty alcohol is a compound of Formula III: CH₃(CH₂)₁₀CH₂(OCH₂CH₂)ₙOH (Formula III), wherein n is 10-25.

8. The method of claim 6, wherein the octylphenoxy alcohol comprises one or more compounds of Formula I: 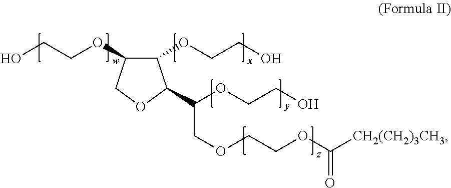
wherein n is 6, 7, 8, 9, or 10, and q is 6, 7, 8, 9, 10, 11, or 12.

9. The method of claim 8, wherein n is 7 or 8 or a mixture thereof, and q is 8; or n is 9 or 10 or a mixture thereof, and q is 8.

10. The method of claim 6, wherein the ethoxylated sorbitan ester is a compound of Formula II: 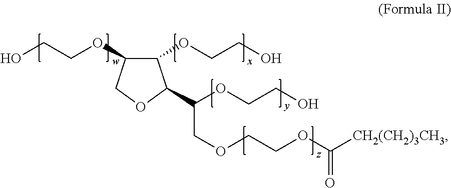
wherein w+x+y+z=20.

11. The method of claim 1, wherein the at least one non-ionic surfactant is selected from the group consisting of Triton X-100, Triton X-114, Brij L23, Tween 20 and IGEPAL CA630.

12. The method of claim 1, wherein the at least one anionic surfactant is selected from the group consisting of sodium cholate, sodium N-lauroyl sarcosine (NLS), sodium dodecyl sulfate (SDS), sodium dodecyl benzene sulfonate (SDBS), and any combination thereof.

13. The method of claim 12, wherein the at least one anionic surfactant is NLS, and the at least one non-ionic surfactant is selected from the group consisting of:
(i) the compound of Formula I,
wherein n is 7 or 8 or a mixture thereof, and q is 8; or
n is 9 or 10 or a mixture thereof, and q is 8;
(ii) the compound of Formula II,
wherein w+x+y+z=20; and
(iii) the compound of Formula III,
wherein n is 23.

14. The method of claim 12, wherein the at least one anionic surfactant is SDS, and the at least one non-ionic surfactant is selected from the group consisting of:
(i) the compound of Formula I,
wherein n is 7 or 8 or a mixture thereof, and q is 8; or
n is 9 or 10 or a mixture thereof, and q is 8;
(ii) the compound of Formula II,
wherein w+x+y+z=20; and
(iii) the compound of Formula III,
wherein n is 23.

15. The method of claim 12, wherein the at least one anionic surfactant is SDBS, and the at least one non-ionic surfactant is selected from the group consisting of:
(i) the compound of Formula I,
wherein n is 7 or 8 or a mixture thereof, and q is 8; or
n is 9 or 10 or a mixture thereof, and q is 8;
(ii) the compound of Formula II,
wherein w+x+y+z=20; and
(iii) the compound of Formula III,
wherein n is 23.

16. The method of claim 12, wherein the at least one anionic surfactant is sodium cholate, and the at least one non-ionic surfactant is selected from the group consisting of:
(i) the compound of Formula I,
wherein n is 9 or 10 or a mixture thereof, and q is 8;
(ii) the compound of Formula II,
wherein w+x+y+z=20; and
(iii) the compound of Formula III,
wherein n is 23.

17. The method of claim 1, wherein the lysing composition further comprises one or more of a pH buffer, salt, proteinase, or combination thereof; wherein the pH buffer is Tris-HCl and has a concentration of about 0.3-3M; wherein the salt is selected from the group consisting of NaCl, KCl, NH₄Cl, Na₂SO₄, K₂SO₄, (NH₄)₂SO₄, guanidinium hydrochloride, and guanidinium thiocyanate; wherein the salt has a concentration of about 0.3-3M; and wherein the proteinase is selected from the group consisting of Arg-C proteinase, BNPS-Skatole, Caspase3, Caspase6, Caspase9, Chymotrypsin-high specificity, Clostripain, Factor Xa, GranzymeB, LysC, Neutrophil elastase, Pepsin, Proteinase K, Thermolysin, Asp-N endopeptidase, Caspase1, Caspase4, Caspase7, Caspase10, Chymotrypsin-low specificity, CNBr, Formic acid, Hydroxylamine, LysN, Staphylococcal peptidase I, Thrombin, Asp-N endopeptidase+N-terminal Glu, Caspase2, Caspase5, Caspase8, Enterokinase, Glutamyl endopeptidase, Iodosobenzoic acid, 2-nitro-5-thiocyanobenzoic acid, Proline-endopeptidase, Tobacco etch virus protease, and Trypsin.

18. The method of claim 1, wherein when the at least one non-ionic surfactant and the at least one anionic surfactant are dissolved in a volume of water, the anionic surfactant is present at a concentration of about 0.03M to 0.4M, and the non-ionic surfactant is present at a concentration of about 1-20% (v/v).

19. A lysing composition, comprising:
(a) at least one non-ionic surfactant with a hydrophilic-lipophilic balance (HLB) value between 10 and 20; and
(b) at least one anionic surfactant, wherein the anionic surfactant comprises a cation and an anionic group; wherein the anionic group is selected from the group consisting of a cholate, a carboxylate, a sulfonate, a sulfate, and a phosphate ester; and the cation is selected from the group consisting of sodium, potassium, ammonium, calcium, lithium, magnesium, aluminum, cesium, barium, straight trimethyl ammonium, branched trimethyl ammonium, triethyl ammonium, tripropyl ammonium, tributyl ammonium, tetramethyl ammonium, tetraethyl ammonium, tetrapropyl ammonium, and tetrabutyl ammonium,
wherein the at least one non-ionic surfactant and the at least one anionic surfactant are present in a ratio such that when the at least one non-ionic surfactant and the at least one anionic surfactant are dissolved in a volume of water, the anionic surfactant is present at a concentration of about 0.5-20% (w/v), and the non-ionic surfactant is present at a concentration of about 0.5-20% (w/v).

20. A kit comprising:
(a) the lysing composition of claim 19; and
(b) at least one aqueous two-phase system (ATPS) composition, wherein the at least one aqueous two-phase system (ATPS) comprises a polymer, a salt, a surfactant, or any combination thereof.
